(19) United States
(12) Patent Application Publication
(10) Pub. No.: US 2025/0222175 A1
(43) Pub. Date: Jul. 10, 2025

(54) IMPLANTABLE TISSUE SCAFFOLD

(71) Applicant: W. L. Gore & Associates, Inc., Newark, DE (US)

(72) Inventors: Kenneth E. Berkery, Pearl River, NY (US); Kerry Brunner, Flagstaff, AZ (US); Kirsten N. Cicotte, Maineville, OH (US); Austin J. Crouse, Landenberg, PA (US); Jessica M. Crouse, Landenberg, PA (US); John M. Herman, Elkton, MD (US); Matthew A. Johnson, Bear, DE (US); Maria J. Miller, Phoenix, AZ (US); Joseph B. Koenig, Flagstaff, AZ (US); Stephen Ralph, Phoenix, AZ (US)

(21) Appl. No.: 19/009,283

(22) Filed: Jan. 3, 2025

Related U.S. Application Data
(60) Provisional application No. 63/689,994, filed on Sep. 3, 2024.
(60) Provisional application No. 63/617,991, filed on Jan. 5, 2024.
(60) Provisional application No. 63/691,810, filed on Sep. 6, 2024.

(51) Int. Cl. A61L 27/56; A61F 2/00; A61F 2/12; A61L 27/26; A61L 27/58

(52) U.S. Cl. CPC A61L 27/56; A61F 2/12; A61L 27/26; A61L 27/58; A61F 2002/0081; A61F 2210/0004; A61F 2210/0061

(57) ABSTRACT

A material includes an implantable substrate defined by at least one inelastic filament, wherein the implantable substrate is porous and includes recoverable stored length when free of elastomeric materials. The at least one inelastic filament is bioabsorbable.

### FIELD

The present disclosure relates generally to apparatuses, systems, and methods relating to implantable substrates having stored length. Specifically, the disclosure relates to apparatuses, systems, and methods relating to implantable substrates having stored length and a three-dimensional porous structure that can be provided in various shapes including three-dimensional shapes.

### BACKGROUND

Currently available implantable, biodegradable substrates comprise relatively inelastic fibers resulting in a substrate that has little to no elastic properties. This is especially problematic where the substrate, for example, is in the form of a thin sheet that is used to conform to three-dimensional tissue and/or implant surfaces resulting in wrinkles and/or folds where a smooth profile is desired.

### SUMMARY

Implantable substrates are provided to have stored length, including recoverable stored length. The implantable substrates as discussed herein may be used in various applications, including but not limited to surgical applications. For example, the implantable substrates may be used in conjunction with tissue expanders and implants for breast reconstruction. In some embodiments, the implantable substrates are biodegradable. The implantable substrates have recoverable stored length which imparts bulk material elasticity without the addition of structural components, such as, but not limited to, an elastomer or elastomeric material, to impart an elastic property to the implantable substrate.

According to an example (Example 1), a material includes an implantable substrate defined by at least one inelastic filament, wherein the implantable substrate is porous and includes recoverable stored length when free of elastomeric materials.

According to another example (Example 2), further to Example 1, the implantable substrate includes from about 30% to about 160% stored length.

According to another example (Example 3), further to any of the preceding Examples, the implantable substrate is at least biaxially expandable at room temperature and body temperature.

According to another example (Example 4), further to any of the preceding

Examples, the implantable substrate defines pleats.

According to another example (Example 5), further to Example 4, the pleats are positioned from about 0.00 mm to about 3.00 mm apart from each other.

According to another example (Example 6), further to any one of Examples 4-5, at least some of the pleats intersect.

According to another example (Example 7), further to any one of Examples 4-6, at least one of the pleats include a non-uniform pattern.

According to another example (Example 8), further to any one of Examples 4-7, the pleats define a network of pleats.

According to another example (Example 9), further to any one of Examples 1-8, the pleats are configured to provide the recoverable stored length such that the pleats are configured to substantially return to an original configuration.

According to another example (Example 10), further to Example 9, the pleats are configured to limit macro-wrinkling and macro-folding of the implantable substrate when at least a portion of the recoverable stored length is recovered.

According to another example (Example 11), further to any one of Examples 1-10, the at least one inelastic filament includes melt-formed continuous filaments intermingled to form a porous web, wherein the melt-formed continuous filaments are self-cohered to each other at multiple contact points.

According to another example (Example 12), further to Example 11, the melt-formed continuous filaments comprise at least one semi-crystalline polymeric component covalently bonded to or blended with at least one amorphous polymeric component.

According to another example (Example 13), further to Example 12, the melt-formed continuous filaments possess partial to full polymeric component phase immiscibility when in a crystalline state.

According to another example (Example 14), further to any one of Examples 1-13, the implantable substrate has a porosity greater than 70%.

According to another example (Example 15), further to any one of Examples 1-14, the implantable substrate includes a three-dimensional porous structure.

According to another example (Example 16), further to Example 15, the three-dimensional porous structure is at least 0.1 mm thick.

According to another example (Example 17), further to any one of Examples 15-16, the three-dimensional porous structure includes an interconnected pore structure through a thickness of the material.

According to another example (Example 18), further to any one of Examples 1-17, the material defines a three-dimensional shape.

According to another example (Example 19), further to Example 18, the three-dimensional shape is at least one of a tubular construct, a sphere, a hemisphere, a partial sphere, a spheroid, a hemispheroid, a partial spheroid, an ellipsoid, a hemi-ellipsoid, a partial ellipsoid, a cone, and a partial dome.

According to another example (Example 20), further to any one of Examples 1-17, the material defines a substantially planar structure.

According to another example (Example 21), further to any one of Examples 1-20, the material includes at least a first region and a second region, wherein the first region has less stored length than the second region.

According to another example (Example 22), further to any one of Examples 1-21, the material includes a first region and a second region, wherein the first region includes less stored length per cm2 relative to the second region.

According to another example (Example 23), further to any one of Examples 1-22, the at least one inelastic filament is bioabsorbable.

According to an example (Example 24) an implantable substrate includes at least one bioabsorbable polymer defining a tissue scaffold having an anterior side and a posterior side defining a cavity therebetween, the cavity configured to at least partially accommodate an implantable device, wherein at least a portion of the implantable substrate includes recoverable stored length.

According to another example (Example 25), further to Example 24, the posterior side of the tissue scaffold includes a portion of the implantable substrate that is inelastic and the anterior side of the tissue scaffold includes the portion of the implantable substrate that includes recoverable stored length.

According to another example (Example 26), further to any one of Examples 24-25, the anterior side includes a first sheet of the at least one bioabsorbable polymer defining a first tissue scaffold, the first sheet including the recoverable stored length, the posterior side includes a second sheet of the at least one bioabsorbable polymer defining a second tissue scaffold, the second sheet including a portion that is inelastic.

According to another example (Example 27), further to Example 26, the first sheet and the second sheet are coupled to each other by one of at least sonic welding, thermal welding, an adhesive, bonding, suturing, sewing, and a fastening mechanism.

According to another example (Example 28), further to any one of Examples 26-27, an uncoupled portion of the anterior side and the posterior side define an opening through which the implantable device is configured to pass.

According to another example (Example 29), further to any one of Examples 24-28, the posterior side defines a flange.

According to another example (Example 30), further to any one of Examples 24-29, the posterior side defines at least one slit configured to receive a corresponding tab of the implantable device, wherein the flange is configured to cover the corresponding tab of the implantable device.

According to another example (Example 31), further to any one of Examples 24-30, the posterior side defines an aperture.

According to another example (Example 32), further to any one of Examples 24-31, the anterior side includes an extension portion, wherein the extension portion extends beyond a boundary of the posterior side.

According to another example (Example 33), further to Example 32, the extension portion includes one of a rounded or a rectangular profile.

According to an example (Example 34), an implantable substrate includes a first tissue scaffold formed of a bioabsorbable material, wherein the first tissue scaffold includes stored length; and a second tissue scaffold coupled to the first tissue scaffold to define a cavity configured to accommodate an implantable device at least partially therein, the second tissue scaffold including at least a portion that includes less stored length by surface area than the first tissue scaffold.

According to another example (Example 35), further to Example 34, one of the first tissue scaffold and the second tissue scaffold defines a flange configured to cover anchoring tabs of the implantable device when positioned in the cavity.

According to another example (Example 36), further to any one of Examples 34-35, an opening to the cavity is defined between the first and second tissue scaffolds, wherein the second tissue scaffold defines an aperture.

According to another example (Example 37), further to Example 36, the second tissue scaffold includes a cinch portion positioned at least partially about the aperture.

According to another example (Example 38), further to Example 37, the cinch portion includes recoverable stored length.

According to another example (Example 39), further to any one of Examples 34-38, the first tissue scaffold includes an extension portion that extends beyond the at least a portion of an outer perimeter of the second tissue scaffold.

According to another example (Example 40), further to Example 39, the extension portion is configured to extend beyond the implantable device and is configured to be sutured to tissue.

According to another example (Example 41), further to any one of Examples 34-40, the first and second tissue scaffolds are coupled to each other by one of at least sonic welding, thermal welding, an adhesive, bonding, suturing, and a fastening mechanism.

According to an example (Example 42), an implantable medical device for breast reconstruction includes an implantable substrate including a bioabsorbable polymer that is inelastic defining a tissue scaffold including a posterior side and an anterior side, the posterior and anterior side defining a cavity therebetween, the anterior side including an extension portion that includes recoverable stored length; and an implantable device, wherein the cavity of the tissue scaffold is configured to at least partially receive the implantable device.

According to another example (Example 43), further to Example 42, the posterior side includes a flange that includes less stored length by surface area than the first tissue scaffold.

According to another example (Example 44), further to Example 43, the extension portion defines a first length along a first longitudinal axis this is disposed in a first direction and the flange defines a second length along a second longitudinal axis that is disposed in the first direction, wherein the first length is greater than the second length.

According to an example (Example 45), an implantable substrate includes a bioabsorbable polymer that is inelastic defining a tissue scaffold including a posterior side and an anterior side, the posterior and anterior side defining a cavity therebetween, the anterior side including an extension portion that includes recoverable stored length.

According to another example (Example 46), further to Example 45, the posterior side includes a flange that includes less stored length by surface area than the first tissue scaffold.

According to another example (Example 47), further to Example 46, the extension portion defines a first length along a first longitudinal axis this is disposed in a first direction and the flange defines a second length along a second longitudinal axis that is disposed in the first direction, wherein the first length is greater than the second length.

According to an example (Example 48), an implantable medical device includes an implantable substrate including a bioabsorbable polymer that is inelastic defining a tissue scaffold including a posterior side and an anterior side, the posterior and anterior side defining a cavity therebetween, wherein at least a portion of the anterior side includes recoverable stored length; and an implantable device, wherein the cavity of the tissue scaffold is configured to at least partially receive the implantable device.

According to another example (Example 49), further to Example 48, the tissue scaffold includes a first tissue scaffold defining the anterior side and coupled to a second tissue scaffold defining the posterior side, therein the first and second tissue scaffolds are coupled to each other at a coupling position, wherein slits are formed through the tissue scaffold on at least one of the anterior and posterior sides.

According to another example (Example 50), further to Example 49, the second tissue scaffold includes stored length.

According to another example (Example 51), further to Example 50, the slits are defined through at least one of the first tissue scaffold and the second tissue scaffold including stored length.

According to another example (Example 52), further to any one of Examples 49-51, the slits are positioned adjacent to the coupling position.

According to another example (Example 53), further to any one of Examples 49-55, the slits define windows that have a length that is at least 50% of the width of the slits.

According to another example (Example 54), further to any one of Examples 48-53, the implantable substrate includes a flange on one of the anterior and posterior sides.

According to another example (Example 55), further to Example 54, the flange has a variable width at various locations about a periphery of the implantable substrate.

According to another example (Example 56), further to Example 55, the flange is narrower at lower position corresponding to a lower pole.

According to another example (Example 57), further to any one of Examples 54-56, the implantable substrate defines a fold proximate the flange.

According to another example (Example 58), further to Example 57, the fold is positioned on the anterior side of the tissue scaffold such that at least a portion of the flange is concealed by the anterior side when viewed from an anterior position.

According to another example (Example 59), further to any one of Examples 48-58, the tissue scaffold defines a cuff.

According to another example (Example 60), further to Example 59, the cuff is positioned at least partially at a lower portion of the tissue scaffold.

According to another example (Example 61), further to Example 60, the lower portion of the tissue scaffold includes an oblong shape configured to mimic an inframammary fold.

According to another example (Example 62), further to any one of Examples 48-61, the posterior side defines an arc at a portion of a perimeter of the tissue scaffold, wherein the arc extends less than about 19 to about 25 degrees above a horizontal line defined by a center of the arc.

According to another example (Example 63), further to any one of Examples 48-62, the posterior side includes a bar extending across at least a portion of a width of the posterior side.

According to another example (Example 64), further to Example 63, the bar is configured to extend when tension is applied thereto.

According to another example (Example 65), further to any one of Examples 48-64, the posterior side includes an orientation feature for orienting the implantable device with respect to the implantable substrate.

According to another example (Example 66), further to any one of Examples 48-65, the posterior side defines an opening into the cavity that is off-center.

According to another example (Example 67), further to any one of Examples 48-66, the anterior side includes preferential stretch in at least one predefined region of the implantable substrate.

According to another example (Example 68), further to any one of Examples 48-67, the anterior side and the posterior side both include recoverable stored length.

According to an example (Example 69), an implantable substrate includes a bioabsorbable polymer that is inelastic defining a tissue scaffold including a posterior side and an anterior side, the posterior and anterior side defining a cavity therebetween, wherein at least a portion of the anterior side includes recoverable stored length.

According to another example (Example 70), further to Example 69, the tissue scaffold includes a first tissue scaffold defining the anterior side and coupled to a second tissue scaffold defining the posterior side, therein the first and second tissue scaffolds are coupled to each other at a coupling position, wherein slits are formed through the tissue scaffold on at least one of the anterior and posterior sides.

According to another example (Example 71), further to Example 69, the second tissue scaffold includes stored length.

According to another example (Example 72), further to Example 71, the slits are defined through at least one of the first tissue scaffold and the second tissue scaffold including stored length.

According to another example (Example 73), further to any one of Examples 70-72, the slits are positioned adjacent to the coupling position.

According to another example (Example 74), further to any one of Examples 70-73, the slits define windows that have a length that is at least 50% of the width of the slits.

According to another example (Example 75), further to any one of

Examples 70-74, the implantable substrate includes a flange on one of the anterior and posterior sides.

According to another example (Example 76), further to Example 75, the flange has a variable width at various locations about a periphery of the implantable substrate.

According to another example (Example 77), further to Example 76, the flange is narrower at lower position corresponding to a lower pole.

According to another example (Example 78), further to any one of Examples 75-77, the implantable substrate defines a fold proximate the flange.

According to another example (Example 79), further to Example 78, the fold is positioned on the anterior side of the tissue scaffold such that at least a portion of the flange is concealed by the anterior side when viewed from an anterior position.

According to another example (Example 80), further to any one of Examples 69-79, the tissue scaffold defines a cuff.

According to another example (Example 81), further to Example 80, the cuff is positioned at least partially at a lower portion of the tissue scaffold.

According to another example (Example 82), further to Example 81, the lower portion of the tissue scaffold includes an oblong shape configured to mimic an inframammary fold.

According to another example (Example 83), further to any one of Examples 69-82, the posterior side defines an arc at a portion of a perimeter of the tissue scaffold, wherein the arc extends less than about 19 to about 25 degrees above a horizontal line defined by a center of the arc.

According to another example (Example 84), further to any one of Examples 69-83, the posterior side includes a bar extending across at least a portion of a width of the posterior side.

According to another example (Example 85), further to Example 84, the bar is configured to extend when tension is applied thereto.

According to another example (Example 86), further to any one of Examples 69-85, the posterior side includes an orientation feature for orienting the implantable device with respect to the implantable substrate.

According to another example (Example 87), further to any one of Examples 69-86, the posterior side defines an opening into the cavity that is off-center.

According to another example (Example 88), further to any one of Examples 69-87, the anterior side includes preferential stretch in at least one predefined region of the implantable substrate.

According to another example (Example 89), further to any one of Examples 69-88, the anterior side and the posterior side both include recoverable stored length.

According to an example (Example 90) a method of reconstructing a breast includes positioning an implantable substrate in a target position proximate a pectoral muscle in a patient, the implantable substrate including a bioabsorbable polymer that is inelastic defining a tissue scaffold including a posterior side and an anterior side, the posterior and anterior side defining a cavity therebetween, the anterior side including an extension portion that includes recoverable stored length; and positioning an implantable device in the cavity of the tissue scaffold.

According to another example (Example 91), the method of Example 90 further includes expanding the implantable medical device to a larger volume, wherein the implantable medical device includes a tissue expander.

According to another example (Example 92), the method of either Example 90 or 91 further includes coupling at least one of the implantable substrate and the implantable device to tissue of the patient.

According to an example (Example 93), an implantable substrate includes a bioabsorbable fiber that is configured as a knit substrate, wherein the knit substrate defines a cavity including a volume, and an access opening into the cavity, wherein the knit substrate is configured to stretch or expand from a first configuration in which the cavity includes a first volume toward a second configuration in which the cavity includes a second volume that is greater than the first volume, wherein the cavity is configured to at least partially receive an implantable device.

According to another example (Example 94), further to Example 93, the knit substrate exhibits at least one of stretchability, conformability, and expandability as bulk material property.

According to another example (Example 95), further to any one of Examples 93-94, the knit substrate includes a cinch portion surrounding the access opening.

According to another example (Example 96), further to any one of Examples 93-95, a first portion of the implantable substrate includes a first knit pattern and a second portion of the implantable substrate includes a second knit pattern that is different from the first pattern.

According to another example (Example 97), further to Example 96, the first pattern provides a first material property and the second pattern provides a second material property that is different from the first material property.

According to another example (Example 98), further to Example 97, the first material property that is different from the second material property is varying levels of at least one of expandability, stretchability, recoverability, conformability, drapability, or stretchability in a first direction.

According to another example (Example 99), further to any one of Examples 93-98, the bioabsorbable fiber includes at least one filament of glycolide and trimethylene carbonate (PGA:TMC).

According to another example (Example 100), further to Example 99, the bioabsorbable fiber include a plurality of filaments, wherein at least one of the plurality of filaments is defined by a material other than PGA:TMC.

According to another example (Example 101), further to any one of Examples 93-100, the knit substrate includes a bulk three-dimensional shape.

According to another example (Example 102), further to any one of Examples 93-101, the implantable substrate is entirely bioabsorbable.

According to an example (Example 103), an implantable substrate includes a bioabsorbable fiber that is configured as a knit substrate, wherein the knit substrate defines an anterior side, a posterior side, and a cavity therebetween, the knit substrate including an access opening into the cavity, wherein the knit substrate includes a three-dimensional shape and has a bulk material elasticity, wherein the cavity is configured to at least partially receive an implantable device.

According to another example (Example 104), further to Example 103, the anterior side of the implantable substrate may include a first knit pattern and the posterior side of the implantable substrate may include a second knit pattern that is different from the first pattern.

According to another example (Example 105), further to Example 104, the first pattern provides a first material property and the second pattern provides a second material property that is different from the first material property.

According to another example (Example 106), further to Example 105, the first material property that is different from the second material property is varying levels of at least one of stretchability, expandability, recoverability, conformability, drapability, or stretchability in a first direction.

According to another example (Example 107), further to any one of Examples 103-106, the bioabsorbable fiber includes at least one filament of glycolide and trimethylene carbonate (PGA:TMC).

According to another example (Example 108), further to Example 107, the bioabsorbable fiber include a plurality of filaments, wherein at least one of the plurality of filaments is defined by a material other than PGA:TMC.

According to an example (Example 109), a method of manufacturing an implantable substrate includes knitting a knit substrate including a three-dimensional shape and has a bulk material elasticity, the knit substrate defining a cavity and an access opening into the cavity, wherein the cavity is configured to at least partially receive an implantable device.

According to another example (Example 110), further to Example 109, knitting the knitted substrate includes knitting a first portion with a first knit pattern and a second portion with a second knit pattern.

According to another example (Example 111), further to any one of Examples 109-110, the method further includes forming a fiber including a plurality of filaments.

According to another example (Example 112), further to Example 111, the plurality of filaments include PGA:TMC.

According to another example (Example 113), further to any one of Examples 109-112, the method further includes coupling a closure member to the knit substrate proximate the access opening.

According to an example (Example 114), an implantable substrate includes a bioabsorbable fiber that is configured as a knit substrate, wherein the knit substrate defines an anterior side, a posterior side, and a cavity therebetween, the knit substrate including an access opening into the cavity, wherein the knit substrate includes a three-dimensional shape and has a bulk material elasticity, wherein the cavity is configured to at least partially receive an implantable device therein via the access opening.

According to an example (Example 115), a method of reconstructing a breast includes positioning an implantable substrate in a target position proximate a pectoral muscle in a patient, the implantable substrate including a bioabsorbable fiber that is configured as a knit substrate, wherein the knit substrate defines a cavity therein, the knit substrate including an access opening into the cavity, wherein the knit substrate includes a three-dimensional shape and has a bulk material elasticity; and positioning at least a portion of an implantable device in the cavity of the tissue scaffold.

According to another example (Example 116), further to Example 115, the method further includes expanding the implantable device to a larger volume, wherein the implantable device includes one of a tissue expander and a permanent implant.

According to another example (Example 117), further to Examples 115-116, the method further includes coupling at least one of the implantable substrate and the implantable device to tissue of the patient.

According to an example (Example 118), an implantable medical device includes an implantable substrate including a first tissue scaffold defined by a bioabsorbable fiber that is configured as a knit substrate and a second tissue scaffold defined by a bioabsorbable polymer that is inelastic and defines a non-woven structure, the first and second tissue scaffolds defining a cavity; and an implantable device, wherein the cavity of the tissue scaffold is configured to at least partially receive the implantable device.

According to another example (Example 119), further to Example 118, the first tissue scaffold includes a knit pattern configured to facilitate expansion.

According to another example (Example 120), further to any one of Examples 118-119, the first tissue scaffold has a bulk two-dimensional structure in an unexpanded configuration.

According to another example (Example 121), further to any one of Examples 118-120, the second tissue scaffold does not include stored length.

According to another example (Example 122), further to any one of Examples 118-120, the first tissue scaffold is positioned on an anterior side and the second tissue scaffold is positioned on a posterior side.

According to another example (Example 123), further to Example 122, the second tissue scaffold includes stored length.

According to another example (Example 124), further to Example 122, the second tissue scaffold does not include stored length.

According to another example (Example 125), further to any one of Examples 118-120, the first tissue scaffold is positioned on a posterior side and the second tissue scaffold is positioned on an anterior side.

According to another example (Example 126), further to Example 125, the second tissue scaffold includes stored length.

According to an example (Example 127), a method of implantation includes positioning an implantable device at least partially within the implantable substrate of any of the preceding claims such that an anterior portion of the implantable device is covered by the implantable substrate; and placing the implantable device and implantable substrate in a patient.

According to another example (Example 127), further to Example 125, the method further includes passing anchoring tabs of the implantable device through slits defined in the implantable substrate.

According to another example (Example 121), further to any one of Examples 127-128, placing the implantable device and implantable substrate is pre-pectoral.

According to another example (Example 121), further to any one of Examples 127-128, placing the implantable device and implantable substrate is subpectoral.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIGS. 1A-1B are illustrations of a material formed of an implantable substrate and having stored length, in accordance with an embodiment;

FIGS. 1C-1D are images of the material of FIGS. 1A-1B, in accordance with an embodiment;

FIG. 2 illustrates various samples of materials formed of an implantable substrate, in accordance with an embodiment;

FIG. 3 is a scanning electron microscope (SEM) image of a surface of a material, in accordance with an embodiment;

FIG. 4 is a SEM image of a cross section of a material, in accordance with an embodiment;

FIG. 5 is a SEM image of a surface of a material prior to expansion, in accordance with an embodiment;

FIG. 6 is a SEM image of the surface of the material of FIG. 5 after expansion and recovery of at least some of the stored length;

FIGS. 7-9 illustrate a method of preparing a material, in accordance with an embodiment;

FIGS. 10-31 illustrate various three-dimensional structures into which a material may be formed, in accordance with an embodiment;

FIG. 32 is a graph of strain versus normalized force of two different samples of material formed of the same substance, the first sample not having stored length and the second sample having stored length, in accordance with some embodiments;

FIG. 33 is a graph of strain versus normalized force of two different samples of material, the first sample being tuned to have a first percentage of stored length and the second sample being tuned to have a second percentage of stored length, in accordance with some embodiments;

FIG. 34 is a graph of stored length versus thickness of two different samples of material, the first sample having a different percentage of stored length and thickness than the second sample, in accordance with some embodiments;

FIGS. 35A and 35B are SEM images of cross sections of the samples of FIG. 34,

FIGS. 36 and 37 are graphs of strain versus force for a sample before and after an expansion cycle illustrating the sample having recoverable length, in accordance with some embodiments;

FIG. 38 illustrates a method of laying an implantable substrate, in accordance with some embodiments;

FIG. 39 illustrates components used in a method of processing an implantable substrate, in accordance with some embodiments;

FIG. 40 is a SEM image of a cross section of a material with pleats in contact with each other, in accordance with an embodiment;

FIG. 41 is a SEM image of a cross section of a material with macro-pleats and micro-pleats, in accordance with an embodiment;

FIG. 42A illustrates a tissue expander that is in a partially filled state and FIG. 42B illustrates an implantable substrate positioned around and conforming to the tissue expander of FIG. 42A, in accordance with an embodiment;

FIG. 44A is a posterior view of FIG. 43B with an anchoring tab positioned in the implantable substrate and FIG. 44B is a posterior view of FIG. 43B with an anchoring tab positioned outside the implantable substrate; FIG. 44A is a bottom view of FIG. 42B and FIG. 44B is a bottom view of FIG. 43B;

FIG. 45A is top view of an implantable substrate prior to expansion according to an embodiment, and FIG. 45B is a side view of the implantable substrate of FIG. 45A prior to expansion;

FIG. 46A is top view of the implantable substrate of FIGS. 45A and 45B while in an expanded configuration, and FIG. 46B is a side view of the implantable substrate of FIG. 46A while in the expanded configuration;

FIG. 47A is top view of the implantable substrate of FIGS. 45A-46B after expansion and recovery of the stored length, and FIG. 47B is a side view of the implantable substrate of FIG. 47A after expansion and recovery of the stored length;

FIGS. 48-51 illustrate a tissue expander surrounded by an implantable substrate with an extension portion in accordance with an embodiment;

FIGS. 52-54 illustrate an implantable substrate with a first tissue scaffold coupled to a second tissue scaffold, in accordance with an embodiment;

FIG. 55 is an illustration of a tissue expander surrounded by an implantable substrate with an extension portion as implanted in a patient, in accordance with an embodiment;

FIG. 56 is an illustration of a tissue expander surrounded by an implantable substrate having a coupling position between anterior and posterior sides, with slits positioned adjacent to the coupling position, in accordance with an embodiment;

FIG. 57 is an illustration of an implantable substrate with slits for anchoring tabs of an implantable device defined on the anterior side of the implantable substrate, in accordance with an embodiment;

FIGS. 58A-58B are illustrations of an implantable substrate including slits that define windows of for anchoring tabs of an implantable device, in accordance with an embodiment;

FIGS. 59-62 are illustrations of an implantable substrate with a fold that mimics the inframammary fold of natural breast tissue, in accordance with some embodiments,

FIGS. 63-64 are illustrations of an implantable substrate defining a flange that is concealed or partially concealed by an anterior side of the implantable substrate, in accordance with some embodiments;

FIGS. 65-67B are illustrations of posterior sides of an implantable substrate defining opening of various sizes, in accordance with some embodiments;

FIGS. 68A-68B are illustrations of posterior sides of an implantable substrate with various configurations, including a posterior side having stored length, in accordance with some embodiments;

FIGS. 69A-69F are illustrations of posterior sides of an implantable substrate with bars of various configurations;

FIGS. 70A-70C are illustrations of an implantable substrate with a seam between the first and second tissue scaffolds, in accordance with various embodiments;

FIGS. 71 and 72 are illustrations of an implantable substrate with orientation features, in accordance with various embodiments;

FIG. 73 is an illustration of an implantable substrate with a cinch portion, in accordance with an embodiment;

FIGS. 74-75 are illustrations of an implantable substrate with patterned wrinkling in accordance with an embodiment;

FIGS. 76A-76B are illustrations of an implantable substrate with an opening on the posterior side that is offset or off-centered, in accordance with an embodiment;

FIGS. 77-79 are illustrations of an implantable substrate including preferential stretch in predefined regions of the implantable substrate, in accordance with various embodiments;

FIG. 80A is an illustration and FIG. 80B is an image of a front view of an implantable substrate with an implantable device (tissue expander, as shown) positioned therein, wherein the tissue expander is in a partially filled state, in accordance with some embodiments;

FIG. 81A is an illustration and FIG. 81B is an image of a front view of an implantable substrate with an implantable device (i.e., tissue expander) positioned therein, wherein the tissue expander is in a filled state, in accordance with some embodiments;

FIG. 82 is an illustration of a side view of an implantable substrate with an implantable device (i.e., tissue expander) positioned therein, wherein the tissue expander is in a partially filled state, in accordance with some embodiments;

FIG. 83 is an illustration of a side view of an implantable substrate with an implantable device (i.e., tissue expander) positioned therein, wherein the tissue expander is in a filled state, in accordance with some embodiments;

FIG. 84A is an illustration and FIG. 84B is an image of a rear view of an implantable substrate with an implantable device (i.e., tissue expander) positioned therein, wherein the tissue expander is in a partially filled state, in accordance with some embodiments;

FIG. 85A is an illustration and FIG. 85B is an image of a rear view of an implantable substrate with an implantable device (i.e., tissue expander) positioned therein, wherein the tissue expander is in a filled state, in accordance with some embodiments;

FIG. 86 is an illustration of an implantable substrate with an implantable device positioned therein, wherein the implantable substrate includes a cinch and a closure member, in accordance with some embodiments;

FIGS. 87A-87C are illustrations of a knit of an implantable substrate, in accordance with some embodiments;

FIGS. 88A-88C are illustrations of another knit of an implantable substrate, in accordance with some embodiments;

FIGS. 89A-89C are illustrations of another knit of an implantable substrate, in accordance with some embodiments;

FIGS. 90A-90C are illustrations of another knit of an implantable substrate, in accordance with some embodiments;

FIGS. 91A-91C are illustrations of another knit of an implantable substrate, in accordance with some embodiments;

FIG. 92 is an illustration of an aperture in an implantable substrate, in accordance with some embodiments; and

FIGS. 93A-96B are illustrations and images of a hybrid implantable substrate implementing both knit a non-woven material, in accordance with some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein “non-woven” generally refers to a type of substrate made from fibers or filaments that form a web without the filaments being interlaced such as found in knitted or woven fabrics.

As used herein “pleat” generally refers to a portion of substrate that is folded or otherwise positioned back on itself. The term as used herein does not require linear, uniform, overlapping, or even arrangements, although those are within the scope of the use of the term. Furthermore, a pleat need not be held by stitching, pressing or sewing, but may instead be held or defined by the material properties of the substrate.

As used herein “fold” generally refers to a portion of substrate at which the substrate has a change in shape to define the position and/or shape of a pleat.

As used herein “modulus” generally refers to a relationship of a material to its deformation subjected to a load applied to the material. When the ratio of deformation is high relative to an applied load, the modulus is low and the material is generally considered elastic. When the ratio of deformation is low relative to an applied load, the modulus is high and the material is generally considered inelastic.

As used herein “elastic” generally refers to a material property in which the material is able to resume its shape after deformation without applying outside force.

As used herein “inelastic” generally refers a material property in which the material does not return to its shape or general shape after substantial deformation. It is understood that known materials have some degree of elasticity and the term is not to be construed as so limiting to known materials that are generally considered inelastic despite including some limited degree of elasticity at low levels of deformation. Inelastic materials discussed herein are materials with limited elastic behaviors such that the materials typically plastically deform at low deformation levels.

As used herein “micro-pleat” generally refers to a portion of material that is adjacent to at least one fold, where the portion of material has a width defined from or between the fold(s) of less than 1 mm.

As used herein “macro-pleat” generally refers to a portion of material that is adjacent to at least one fold, where the portion of material has a width defined from or between the fold(s) of 1 mm or greater.

As used herein “fiber” generally refers to an elongate structure. A fiber may include a mono-filament structure or a multifilament structure. A fiber may include a filament structure that is provided as a yarn with a continuous filament or with a plurality of filaments. The fiber may be provided as a yarn (e.g., a spun yarn or a filament yarn). When implemented as a yarn, the fiber may include a plurality of filaments that are bundled together. In some embodiments, the bundled filaments may be spun and wound.

As used herein “elastomeric” generally refers a material property in which the material exhibits a greater than 10% elastic strain.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

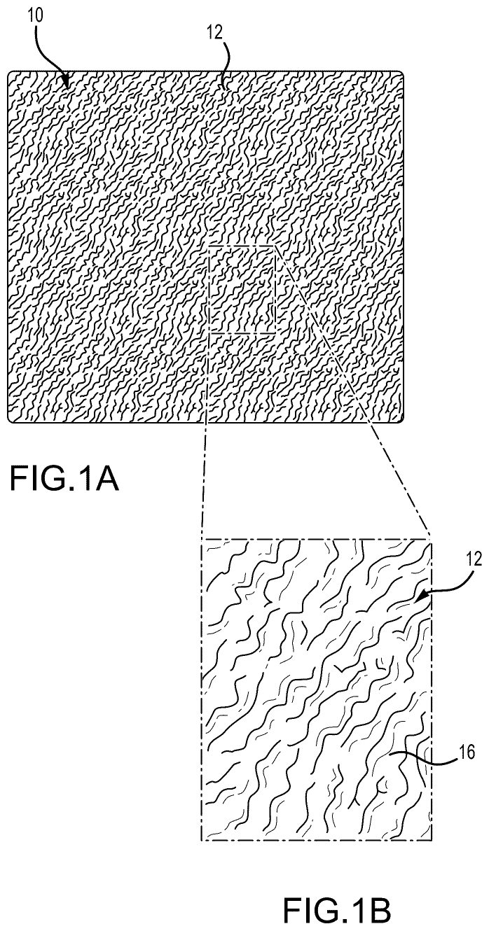

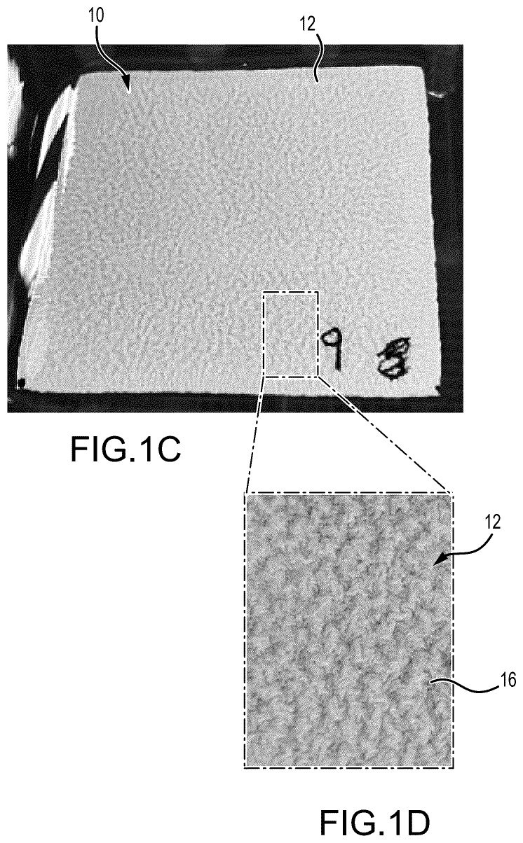

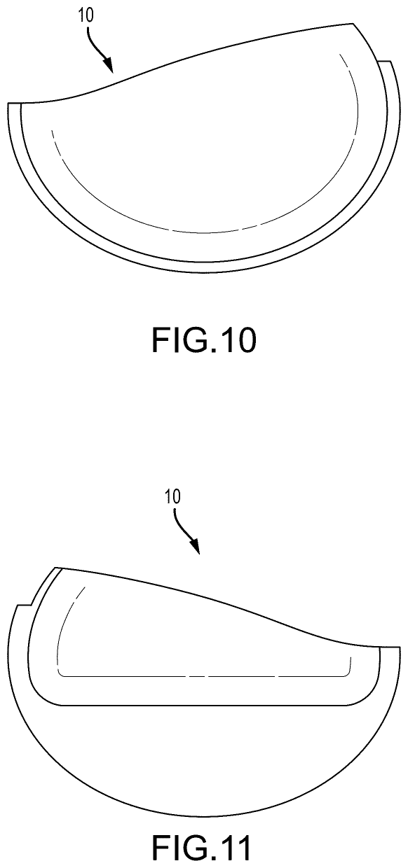

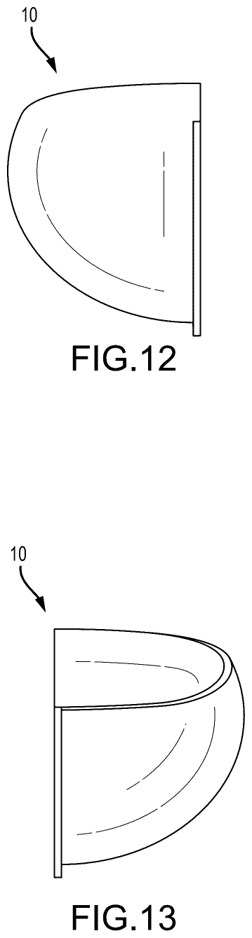

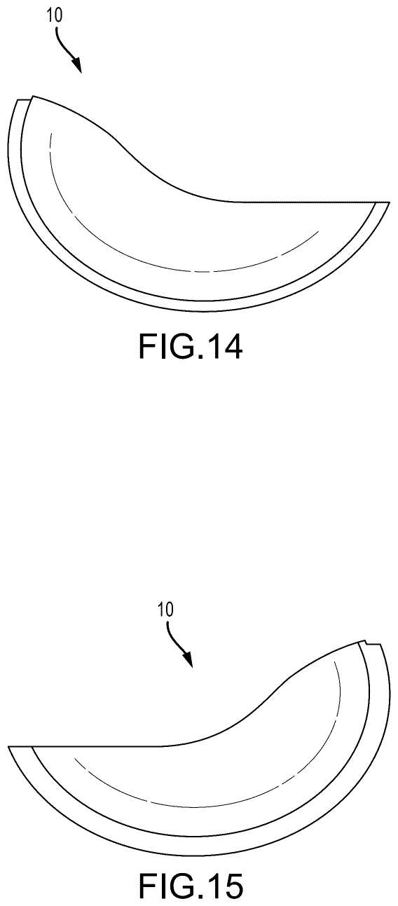

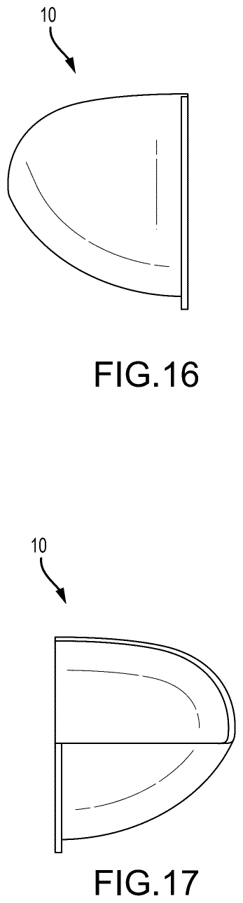

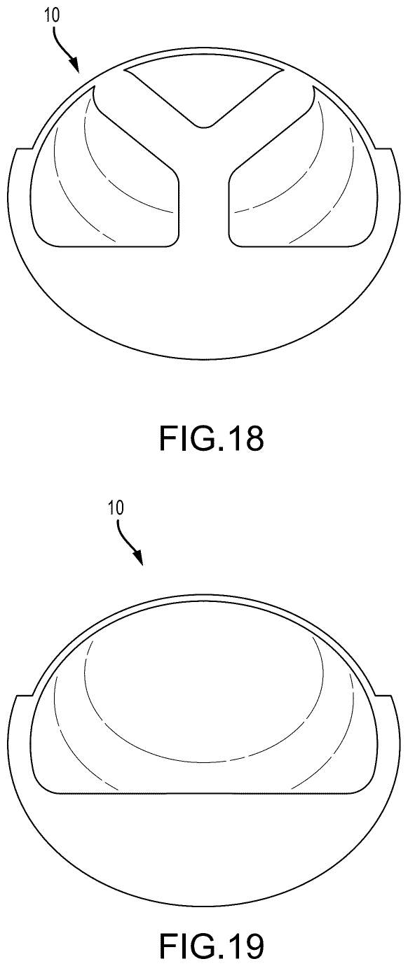

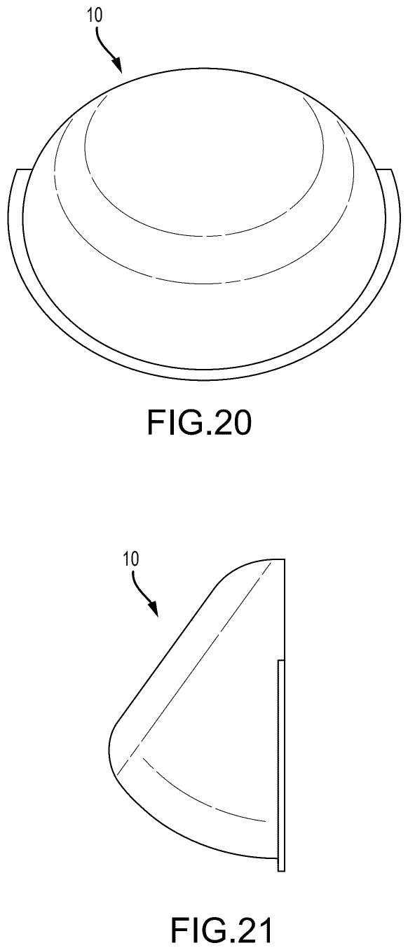

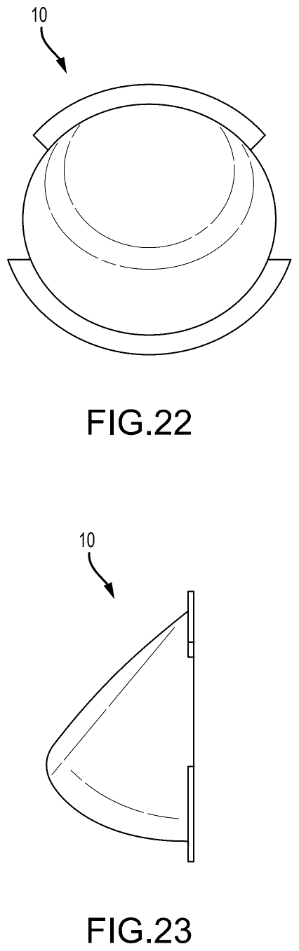

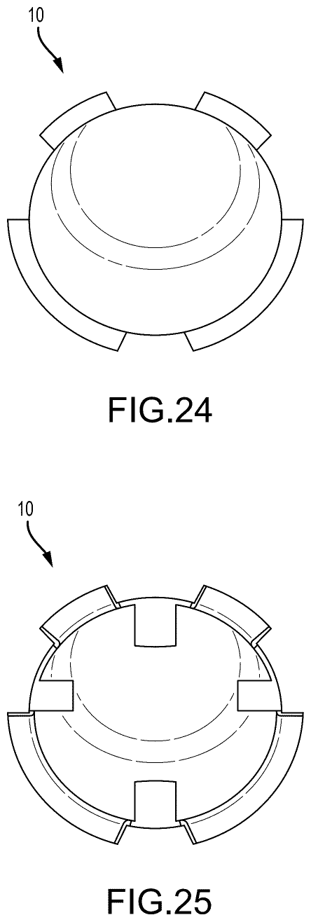

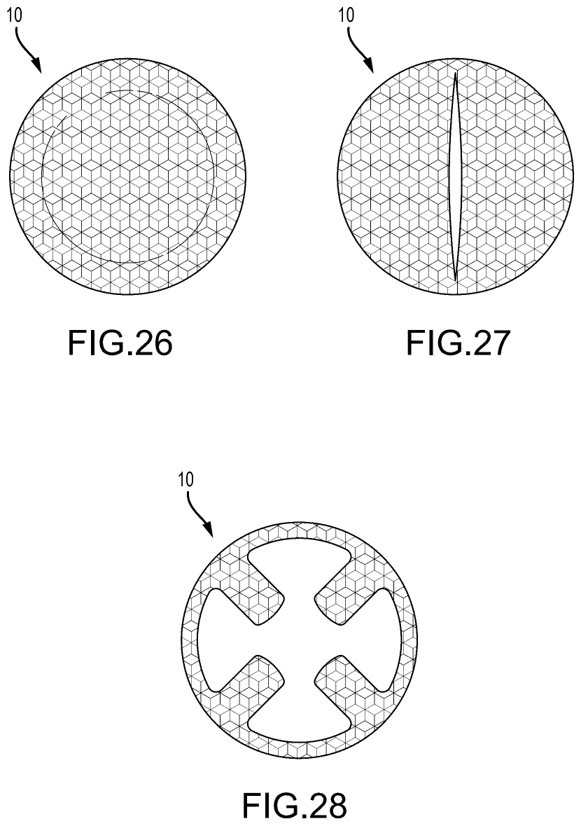

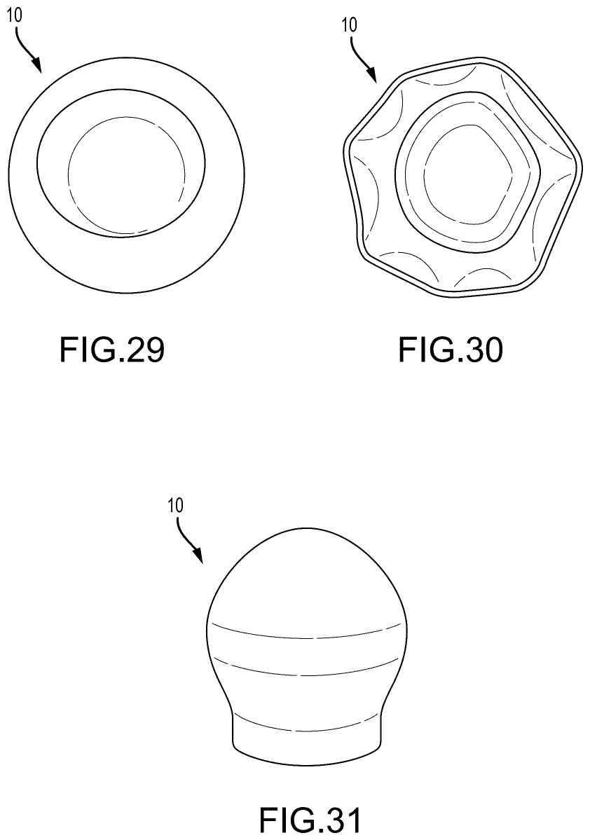

The material or substrate shown in FIG. 1A is provided as an example of the various features of the material and, although the combination of those illustrated features is clearly within the scope of invention, that example and its illustration is not meant to suggest the inventive concepts provided herein are limited from fewer features, additional features, or alternative features to one or more of those features of the material shown in FIG. 1A and may include the other features such as being formed into three-dimensional structures such as shown in FIGS. 10-31. It should also be understood that the reverse is true as well. One or more of the components depicted in FIG. 1A can be employed in addition to, or as an alternative to components depicted in other FIGS.

Discussed herein are materials and methods for forming materials that include an implantable substrate defined by at least one inelastic filament. In accordance with an embodiment, the implantable substrate is defined by at least one inelastic filament defining a non-woven web and comprising only that inelastic filament as the structural component. In accordance with another embodiment, the substrate is defined by a plurality of inelastic filaments defining a non-woven web and comprising only those inelastic filaments as the structural component. It is understood that other materials may be present that do not substantially contribute to the structure or structural properties of the implantable substrate, for example therapeutic coatings. The implantable substrate is porous, includes recoverable stored length (e.g., recoverability) to define a bulk material elasticity, and is free of elastomeric materials. The materials may be implemented in a variety of settings and industries. For example, the materials may be used in the medical industry and in various procedures needing compatible materials that can be formed from inelastic or minimally elastic materials while providing some stored and optionally recoverable length so as to, for example, minimize or prevent wrinkling when conformed to a three-dimensional tissue surface. For example, in breast reconstruction, conformable materials (e.g., stored and optionally recoverable length) may be used to protect tissue and to incorporate tissue during use of a tissue expander or implantable device (e.g., permanent breast implant) with minimal wrinkling. In another example, in breast reconstruction, conformable materials (e.g., stored and optionally recoverable length) may be used to protect tissue and to incorporate tissue during use of an implant. In another example, tubular members may be formed to define lumens through which fluids may flow. The tubular member may be formed of the materials discussed herein in order to provide some tissue incorporation and/or optional coverage and to move and adjust with the tissue during use. More specifically, a vascular graft may be formed with stored length to allow the vascular graft to move with the body and reduce potential trauma to the tissue and reduce potential damage to the graft. More specifically, a tube or patch may be formed with stored length and placed in the gastrointestinal tract to reduce or cover potential trauma, sore, disease, symptom of disease, or surgical intervention and to provide at least some tissue incorporation. In some embodiments, the inelastic filament may include a bioabsorbable material. In some embodiments, the inelastic filament consists of a bioabsorbable material. Various implementations and uses of the materials described herein are contemplated and it is understood that the use cases are not to be limited to any specific procedure or industry.

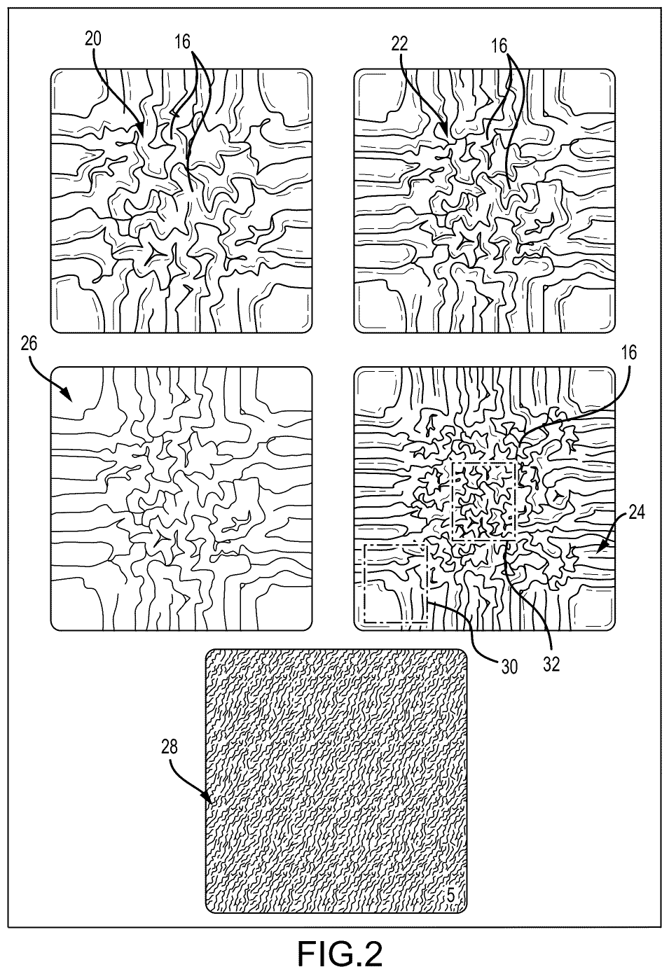

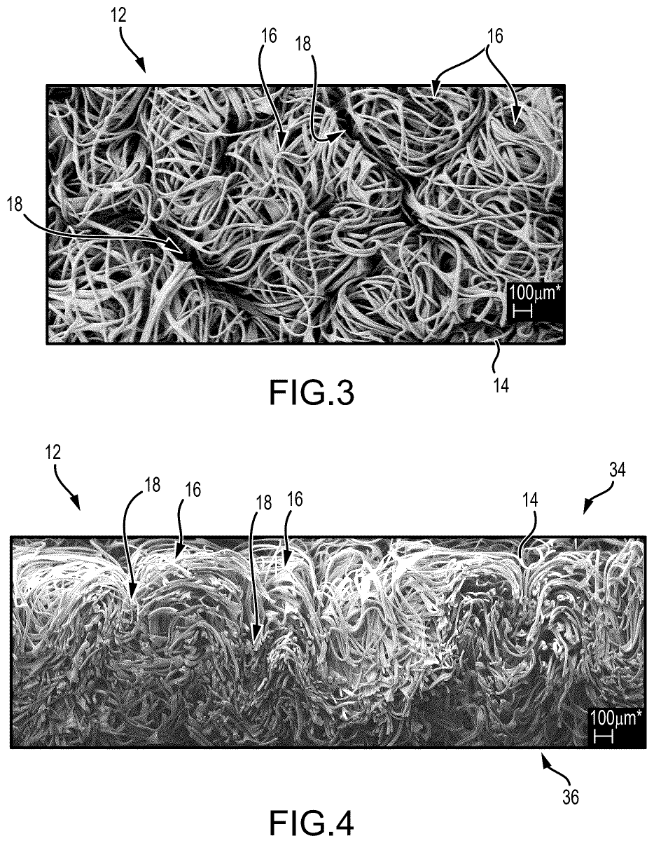

Referring to FIGS. 1A and 1B, a material 10 is illustrated (FIGS. 1C and 1D being images thereof) that includes an implantable substrate 12. FIG. 1B provides a closer view of a portion of FIG. 1A such that some of the surface features may be seen in more detail. FIG. 2 provides various embodiments of the material 10 that may be provided implemented the implantable substrate 12 as described herein. The implantable substrate 12 is defined by at least one inelastic filament 14 (see FIG. 3). The implantable substrate 12 is porous, includes recoverable stored length. The implantable substrate 12 discussed herein is defined by the at least one inelastic filament 14 while having a low elastic modulus bulk material property. The low elastic modulus bulk material property may be obtained when the implantable substrate 12 is free of elastomeric materials (although elastomeric materials may also be incorporated for various reasons without being contrary to the teachings herein). The implantable substrate 12 and specifically the inelastic filament(s) 14 may be formed of various materials and are not necessarily limited to those discussed herein. In some embodiments, the implantable substrate 12 and specifically the inelastic filament(s) 14 may include a bioabsorbable material. For example, the bioabsorbable material may include but are not limited to copolymers and homopolymers of poly (α-hydroxy esters), such as copolymers of poly(lactic-co-glycolic acid) (PLGA), poly(glycolic acid) (PGA), and poly(lactic acid) (PLA); trimethylene carbonate (TMC); copolymers of PLA and TMC (PLA:TMC), copolymers of PGA and TMC (PGA:TMC) and copolymers of PLGA and TMC; and combinations thereof. In some embodiments, the implantable substrate 12 and specifically the inelastic filament(s) 14 may include other inelastic materials that are not necessarily bioabsorbable, for example, polyester. Various other materials may be implemented that exhibit certain properties for facilitating at least some of the properties and functionalities of the implantable substrate 12 after processing. At least some of the properties that facilitate the final functionality and properties of the member implantable substrate 12 are described herein in more detail. It is understood that the implantable substrate 12 may be provided in various forms, including but not limited to non-woven substrates as discussed hereafter (e.g., self-cohering, continuous filament non-woven webs), woven substrates, and so forth.

The material 10 described herein includes various properties that make it useful in a variety of applications. For example, the implantable substrate 12 (e.g., a non-woven bioabsorbable member) includes from about 30% to about 200% stored length or from about 30% to about 160%. The amount of stored length is tunable during processing to facilitate the ability to provide for different amounts of stretch or expansion during use. FIG. 2 illustrates a first sample 20, a second sample 22, a third sample 24, a fourth sample 26, and a fifth sample 28. Each of the samples 20, 22, 24, 26, 28 have been processed to include different surface textures. The surface texture is defined by pleats 16 (e.g., macro-pleats and micro-pleats). Some samples may include both macro-pleats and micro-pleats (e.g., samples 20, 22, 24, 26) whereas other samples may only include micro-pleats (e.g., sample 28). The pleating facilitates storing length of the implantable substrate 12 by positioning the implantable substrate 12 in such a way that the material defining the pleats can be straightened as tension is applied across the implantable substrate 12 or as any other force (e.g., a force applied at an angle non-parallel to the surface of the implantable substrate 12) causes the implantable substrate 12 to release the stored length. In some embodiments, the implantable substrate 12 includes from about 30% to about 40% stored length, from about 40% to about 50% stored length, from about 50% to about 60% stored length, from about 60% to about 70% stored length, from about 70% to about 80% stored length, from about 80% to about 90% stored length, from about 90% to about 100% stored length, from about 100% to about 110% stored length, from about 110% to about 120% stored length, from about 120% to about 130% stored length, from about 130% to about 140% stored length, from about 140% to about 150% stored length, from about 150% to about 160% stored length, from about 160% to about 170% stored length, from about 170% to about 180% stored length, from about 180% to about 190% stored length, and from about 190% to about 200% stored length, or any value or range between the foregoing values, for example.

In some embodiments, the implantable substrate 12 is at least biaxially expandable at room temperature. This may be facilitated by the pleating of the implantable substrate 12 being provided in such a way that length is stored not only in a single direction. For example, referring to FIG. 2, the macro-pleats of the first sample 20 can be seen oriented in various directions which allows stored length to be released in various directions. Biaxial expansion may be desired in certain instances. For example, biaxial expansion may be desirable when the material 10 is anchored substantially around a periphery of the material 10 and the material 10 is being expanded by application of a force along one of the faces of the material 10. By providing biaxial expansion, stress at anchor points may be reduced during expansion as compared to uniaxial expansion. For example, in breast reconstruction, if the material 10 is used in conjunction with a tissue expander and the material 10 is anchored to the patient's surrounding tissue, the tissue expander may be inflated and the material 10 is capable of expanding to include greater length in both the X- and Y-axes along the face of the material. This may include advantages over uniaxial expansion of other materials that may create stress to the tissue at anchor points. This is because biaxial expansion may reduce the amount of released stored length along any single axis in order to accommodate the same amount of expansion of the tissue expander as compared to uniaxial expansion. However, in some embodiments or implementations, it may be advantageous to have only uniaxial expansion. It is understood that processes for preparing the material 10 discussed herein may also be implemented to process the implantable substrate 12 such that it is uniaxially expandable at room temperature and body temperature. In some embodiments, the pleats 16 are configured to limit macro- wrinkling and macro-folding of the implantable substrate 12 when at least a portion of the recoverable stored length is recovered. Additionally, the pleats may be provided in a uniform pattern, a non-uniform pattern, or a combination thereof. This means that the pleats may be provided in a substantially similar size and shape across the surface of the implantable substrate 12, having varying size and shape across the surface of the implantable substrate 12, or having areas of similar size and shape across the surface of the implantable substrate 12 and areas with varying size and shape across the surface of the implantable substrate 12.

In some embodiments, the implantable substrate 12 may define regions of greater and lesser stored length. This can be tuned in the processes described herein. For example, the material 10 may include at least two regions (e.g., a first region 30 and a second region 32) having differing properties. The first region 30 may include less stored length per cm² than the second region 32. This allows for the various regions to preferentially act as supports with less or without expansion and other regions to expand or expand more during use. For example, in breast reconstruction, tissue expanders may be implemented to create space in the chest for implants after a mastectomy. The material 10 may be implanted to protect surrounding tissue while the tissue expander is being used to create the space for the implant, which is filled over time to expand the surrounding tissue. As the tissue expander is filled, the material 10 is capable of expanding with the tissue expander. With controlled expansion in regions, the material is able to support the tissue expander in such a way that reflects natural anatomy. For example, the material 10 may be implanted in such a way that the first region 30 is positioned at a lower pole of the breast to limit expansion of the space downward whereas the second region 32 may have relatively more stored length such that the tissue expander can expand the tissue preferentially outward for more projection or toward the upper pole of the breast. This may be desirable when the material 10 is being used to support or to provide additional support to the tissue expander such that the breast does not unduly expand downward. In other embodiments, the regions may be provided at different orientations to provide other benefits or outcomes. For example, the patient may desire expansion toward the centerline of the body. Additionally, different systems or components may be implemented for similar applications and thus tuning of the regions may be specific to the systems or components being implemented. For example, some tissue expanders may benefit from the material 10 having regions that provide additional support at various positions as expansion may be uneven throughout the expansion profile of the tissue expander. In another example, tissue expanders may benefit from the material 10 tuned with regions that facilitate a more even application of force to the tissue to reduce the likelihood of stretch marks. In another example, tissue expanders may benefit from the material 10 having regions that are capable of expansion at areas where tissue expanders may result in relative movement between the tissue expander and the material 10 if the material 10 did not otherwise have a certain amount of stored length, thus decreasing the amount of surface movement, friction, and potential damage to tissue during expansion. Additionally, the material 10 including regions of variable stored length is contemplated for various other applications other than breast reconstruction or surgical applications. It is understood that the amount of stored length in each region and the number of regions can be tuned to specific applications.

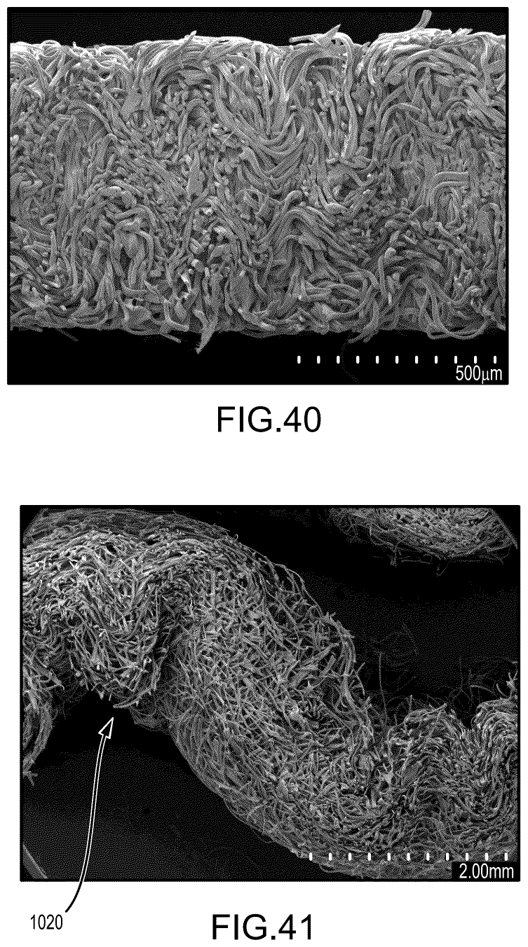

Turning to a more specific discussion of the stored length, the implantable substrate 12 defines pleats 16 (e.g., macro or micro-pleats) which store length and which facilitate expansion of the material 10. The pleats 16 can be seen in the various samples 20, 22, 24, 26, 28 of FIG. 2, the pleats 16 including varied sizes and spacing. Referring to FIGS. 3 and 4, a scanning electron microscope was used to see the structure of one embodiment of the material 10. FIG. 3 shows a surface image of one embodiment of the material 10 and FIG. 4 shows a cross section image of one embodiment of the material 10. As seen in FIG. 3, the material 10 includes micro-pleats (i.e., pleats 16) that are positioned about 0.05 mm to about 0.15 mm from each other. The pleats are defined in this image between folds 18 represented by the spaces. The folds 18 illustrate the implantable substrate 12 turning inward (partially or fully) within a Z-axis of the material 10 (e.g., thickness of the material). Portions of the material 10 positioned within the folds 18 are configured to unfold or be pulled out such that those portions define an outer surface of the material. The pleats 16 and folds 18 can further be seen In FIG. 4, which illustrates the folds 18 representing positions where the implantable substrate 12 turns inward. The folds 18 at the surface as shown in FIG. 3 can define a space between surfaces of the implantable substrate 12 at the surface of the material 10 from about 10 microns to about 100 microns. As can be seen in FIG. 4, pleats 16 are formed on both the inner surface 34 and outer surface 36. In some embodiments, having pleats on the inner surface 34 and outer surface 36 may result in the implantable substrate 12 being positioned to include a substantially serpentine or “S” shape. The serpentine shape is largely defined by the filaments 14. The serpentine shape may not necessarily be defined by a single filament 14 or length of a filament 14, but may be taken as an average of the filament(s) 14 over a length of the implantable substrate 12. In various embodiments, the space between pleats 16 may be minimal (e.g., at least portions of the pleat are in contact with each other) and the space may be up to about 2,000 microns. For example, FIG. 40 illustrates pleats 16 that are in contact with each other and FIG. 41 illustrates pleats 16 that are about 2 mm apart. It is noted that FIG. 41 also illustrates micro-pleats present on macro-pleats as previously discussed.

As discussed, the pleats 16 and regions of the material 10 may be tuned to have different amounts of stored length. Thus, the examples provided with respect to FIGS. 3 and 4 are just that, examples. Other ranges for distances between pleats can be from about 0 microns to about 10 microns, from about 10 microns to about 20 microns, from about 20 microns to about 30 microns, from about 30 microns to about 40 microns, from about 40 microns to about 50 microns, from about 50 microns to about 60 microns, from about 60 microns to about 70 microns, from about 70 microns to about 80 microns, from about 80 microns to about 90 microns, from about 90 microns to about 100 microns, from about 0 microns to about 100 microns, from about 0 microns to about 500 microns, from about 0 microns to about 1,000 microns, and from about 0 microns to about 2,000 microns. In some embodiments, the width of a pleat 16 (i.e., the distance from one edge of a single pleat to the other edge of that same pleat) ranged from about 150 microns to about 6,500 microns. For example, the width of a pleat 16 may be from about 150 microns to about 500 microns, from about 500 microns to about 1,000 microns, from about 1,000 microns, to about 1,500 microns, from about 1,500 microns to about 2,000 microns, from about 2,000 microns to about 3,000 microns, from about 3,000 microns to about 4,000 microns, from about 4,000 microns to about 5,000 microns, and from about 5,000 microns to about 6,500 microns. FIGS. 2, 5, 6, 35A, 35B, 40, and 41 all show embodiments in which average distances between pleats are shown.

Turning to FIGS. 3, 5, and 6, in some embodiments, the pleats 16 and folds 18 may intersect each other. The intersections of the pleats 16 are a result of the shape and orientation of the pleats 16. For example, the pleats 16, in some embodiments, may not extend in a straight course along the surface of the implantable substrate 12. Instead, the pleats may take a non-linear or non-straight path across the surface of the implantable substrate 12. In some instances, a pleat 16 may not extend fully across a surface of the implantable substrate 12, but instead extend across only a portion of the implantable substrate 12. The pleats 16 may intersect each other such that they combine into a single pleat 16 or such that one pleat terminates at a position of a second pleat. The pleats 16 can include any number of shapes and orientations including “S” shaped, lobed, irregular, linear, and so forth. In some embodiments, the shapes are irregular but include substantially similar sizes. In some instances, the surface of the implantable substrate 12 may have the appearance of the folds or wrinkles of the surface of a human brain (e.g., similar shapes to sulci and gyri) or the surface of a walnut. In these embodiments, the pleats 16 and folds 18 may be positioned on an implantable substrate 12 that defines a two-dimensional shape (e.g., a flat sheet) or a three-dimensional shape. Stated otherwise, the pleats 16 and folds 18 define a network or web of pleats 16 and folds 18. The intersections and shapes of the pleats 16 and folds 18 as described facilitate stored length in the material in the X-axis (defined generally by the surface of the material 10) and the Y-axis (defined generally by the surface of the material 10), and combinations thereof.

Because the stored length is facilitated by the pleats 16 and folds 18, it is generally understood that during expansion or release of the pleats 16 and folds 18 as previously discussed, the thickness of the material 10 decreases as the stored length is released. However, during release of the stored length, the thickness or structure of the implantable substrate 12 is generally consistent. For example, the implantable substrate 12 may include a microstructure defined by the filaments 14, where the microstructure is substantially uncollapsed during expansion. This allows the implantable substrate 12 to retain many of its properties and functionalities such as porosity, pore size, plushness, cellular ingrowth, and so forth. The microstructure of the implantable substrate 12 is described in more detail hereafter.

In some embodiments, in order to define the pleats 16 and folds 18 of the implantable substrate 12, the filaments 14 defining the implantable substrate 12 may include melt-formed continuous filaments intermingled to form a porous web, wherein the melt-formed continuous filaments are self-cohered to each other at multiple contact points. Those filaments 14 may be laid to form both the microstructure of the implantable substrate 12 as well as the pleats 16 and folds 18 (e.g., via three-dimensional printing) or the microstructure may be formed and then the implantable substrate 12 may be processed according to methods discussed herein to define the pleats 16 and folds 18. The melt-formed continuous filaments comprise at least one semi-crystalline polymeric component covalently bonded to or blended with at least one amorphous polymeric component. The melt-formed continuous filaments possess partial to full polymeric component phase immiscibility when in a crystalline state. This, in some embodiments, once the pleats 16 and folds 18 are defined by the implantable substrate 12, the implantable substrate 12 can be processed or treated to instigate the crystalline state of at least some of the filaments 14. Otherwise stated, the orientation of the filaments 14 can be set in the crystalline state to substantially retain the shape of the pleats 16 and folds 18.

In some embodiments, the implantable substrate 12 may be provided with various porosities. For example, in some embodiments, the implantable substrate 12 may have a percent porosity greater than 70%, greater than 80%, and greater than 90%. The porosity of the implantable substrate 12 is defined within the three-dimensional microstructure. Because the implantable substrate 12 includes a thickness that is greater than a single filament 14 or even multiple stacked filaments, the pores are defined through a thickness of the implantable substrate 12. In some embodiments, the implantable substrate 12 may be provided with various thicknesses. For example, in some embodiments, the implantable substrate 12 may be at least 0.10 mm thick, at least 0.20 mm thick, at least 0.30 mm thick, at least 0.40 mm thick, at least 0.50 mm thick, at least 0.60 mm thick, at least 0.70 mm thick, at least 0.80 mm thick, at least 0.90mm thick, at least 1.00 mm thick, at least 2.00 mm thick, at least 3.00 mm thick, at least 4.00 mm thick, or at least 5.00 mm thick. The three-dimensional microstructure of the implantable substrate 12 may include a network of pores throughout, including throughout the thickness of the material 10. Because of the three-dimensional character of the implantable substrate 12 and the porous structure defined therein, the material 10 may provide various functionalities. For example, when used in surgical applications, the three-dimensional porous structure may facilitate tissue ingrowth and incorporation, which can provide for faster healing, increased adhesion, and so forth. The three-dimensional porous structure may be provided in such a way that the pores define an interconnected pore structure where the void spaces of the porous structure are fluidly coupled to each other. In these various examples, the implantable substrate 12 may act as a scaffold. In some embodiments, a thin impermeable barrier (not shown) may be applied to or incorporated into the implantable substrate 12 to limit fluid transfer therethrough while also maintaining the functionality of the three-dimensional porous structure such as tissue ingrowth. For example, in some embodiments, a thin, impermeable film may be applied between layers of the implantable substrate or may be applied to one side of the implantable substrate.

In addition to the implantable substrate 12 including a three-dimensional porous structure through the thickness (e.g., Z-axis), in some embodiments the material 10 can be provided with a three-dimensional shape. For example, the material 10 may define a three-dimensional shape useful for the application in which it is to be used. Examples of three-dimensional shapes are provided in FIGS. 10-31. For example, the material 10 shown in FIGS. 10-31 may be implemented in breast reconstructions for supporting tissue expanders or implants. The three-dimensional shape is at least one of a tubular construct, a sphere, a hemisphere, a partial sphere, a spheroid, a hemispheroid, a partial spheroid, an ellipsoid, a hemi-ellipsoid, a partial ellipsoid, a cone, and a partial dome. In other embodiments, the material 10 may be provided as a sheet having a substantially planar structure.

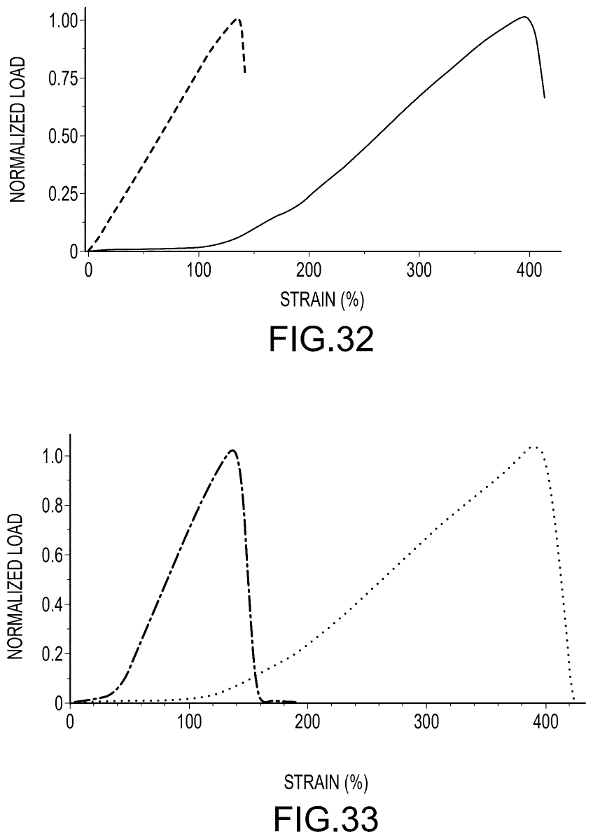

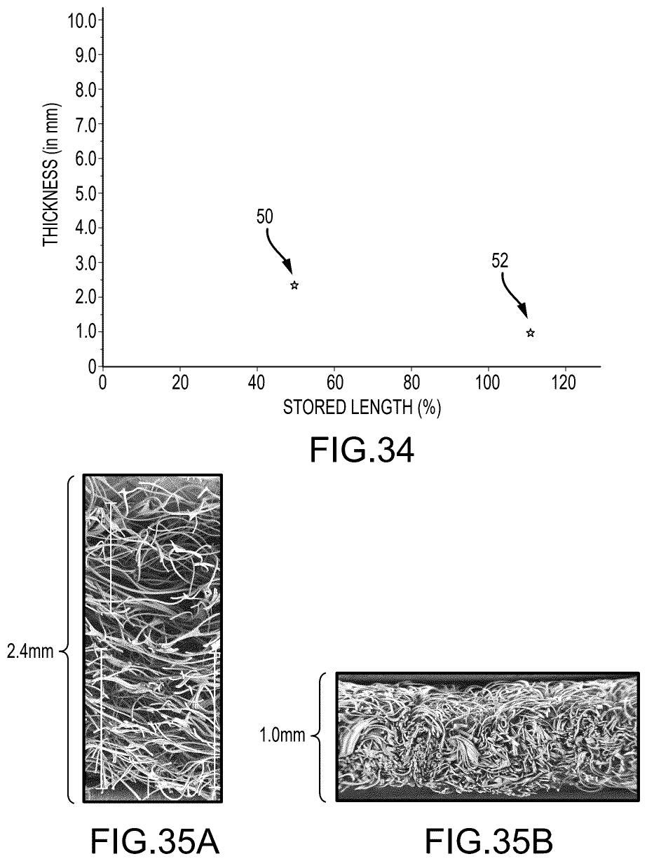

Turning to a discussion of FIGS. 32-37, various properties of the stored length previously discussed are shown in graphical form. Referring to FIG. 32, a graph is provided illustrating the stored length of the material 10 versus a substrate without stored length but formed of a similar material. The substrate without stored length shows a linear relationship between strain and normalized load. In contrast, the material 10 with stored length shows a non-linear relationship between strain and normalized load until the stored length is released, at which point the material 10 then exhibits a linear relationship between strain and normalized load. Referring to FIG. 33, two different materials 10 are shown with different amounts of stored length. As previously discussed, the material 10 can be tuned to have different amounts of stored length. For example, the material 10 can be provided with various amounts of stored length such that the material 10 expands to a specific profile before exhibiting the innate tensile strength of the material 10, meaning the expansion of the material 10 represents the releasing of the stored length via releasing of pleats and folds (e.g., the pleats act as living hinges). In a tensile test, the release of the stored length is represented by a toe region until substantially all of the stored length is released, at which point the inelastic property of the filaments are exhibited in a load building region of the tensile test. The material represented by the data on the left of the graph includes relatively less stored length than the material represented by the data on the right of the graph. Other properties may be tuned including density, thickness, and stored length. For example, FIG. 34 shows two different samples that are produced with different densities, thicknesses, and stored length. The first sample 50 shown on the left of the graph has thickness of about 2.4 mm and a stored length of about 50%. The second sample 52 shown on the right of the graph has a thickness of about 1.0 mm and a stored length of about 110%. FIG. 35A shows a cross section of the first sample 50 of FIG. 34 and FIG. 35B shows a cross section of the second sample 52 of FIG. 34. The different densities can be seen in the SEM images of FIGS. 35A and 35B.

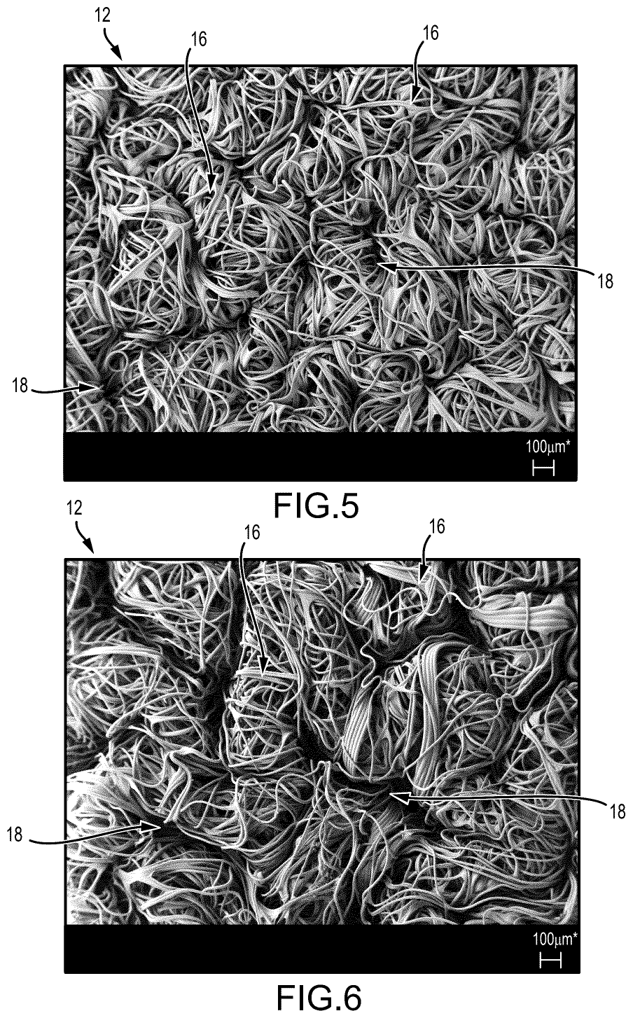

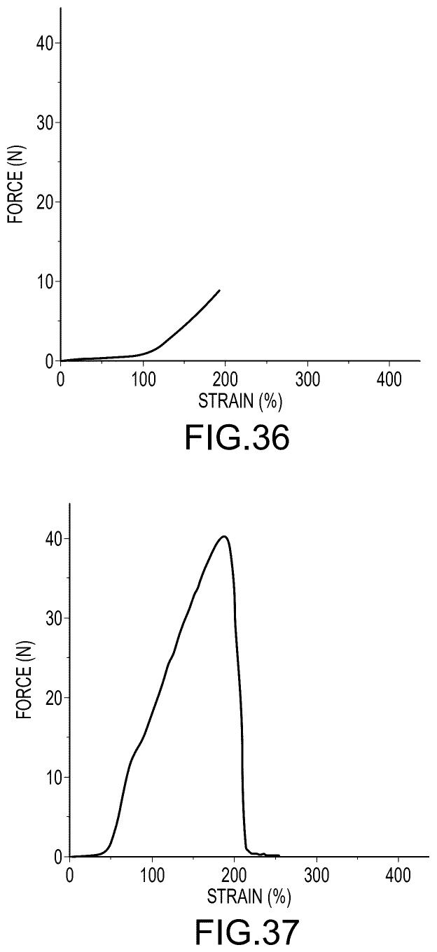

Referring now to FIGS. 36-37, the stored length of the material 10 is illustrated as being recoverable stored length to define a bulk material elasticity. When the material 10 is expanded and released, the material 10 is capable of recovering at least some of the stored length. For example, FIG. 36 shows a sample of material being loaded to about 20% of break load which shows the material experiencing about a 200% strain with about 10 N of force before being released. FIG. 37 illustrated the same sample of material of FIG. 36 then being taken and having a force applied to the material a second time. As can be seen, there is a portion of the curve that is non-linear to about 50% strain. This portion of the curve represents the stored length that the material has even after the first expansion. SEM images of the material 10 prior to expansion and after expansion are provided in FIGS. 5 and 6, respectively. As can be seen, the pleats 16 and folds 18 are at least partially recovered after expansion. For example, in some embodiments, the amount of stored length is from about 30% to about 100%. In some embodiments, not all of the original stored length is recovered after the first expansion, but it was observed that after the first expansion and recovery of stored length, further expansion and release cycles demonstrated that the stored length after the first cycle and any subsequent cycles remains substantially the same. As discussed previously herein, the stored length is understood to be facilitated by the orientation of the filaments 14 and specifically the orientation of the filaments being at least partially locked into the crystalline state.

### EXAMPLES

### Example 1

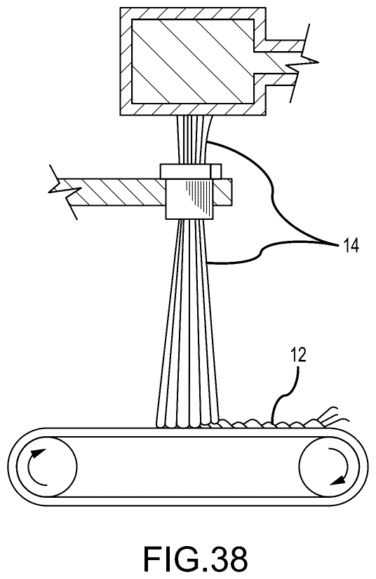

Methods for forming self-cohering, continuous filament non-woven webs include those discussed in U.S. Pat. No. 6,165,217 to W. L. Gore & Associates, which issued on Dec. 26, 2000 and is incorporated herein by reference in its entirety. Bioabsorbable filaments may be implemented in connection with the processes described therein and may result in substrates implemented in further processing as described herein. FIG. 38 illustrates a process of laying down filaments 14 to define the implantable substrate 12.

### Example 2

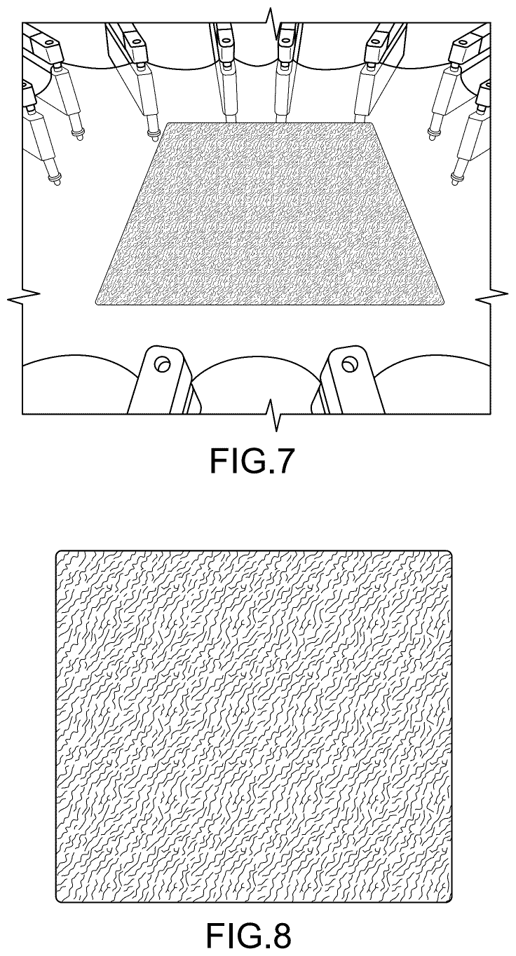

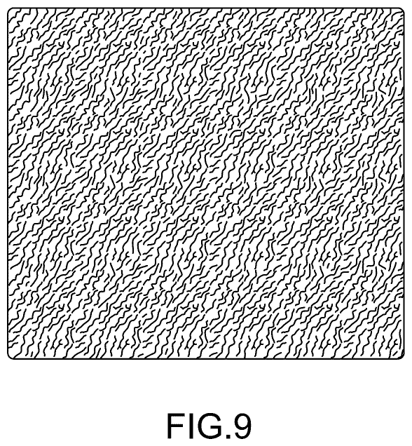

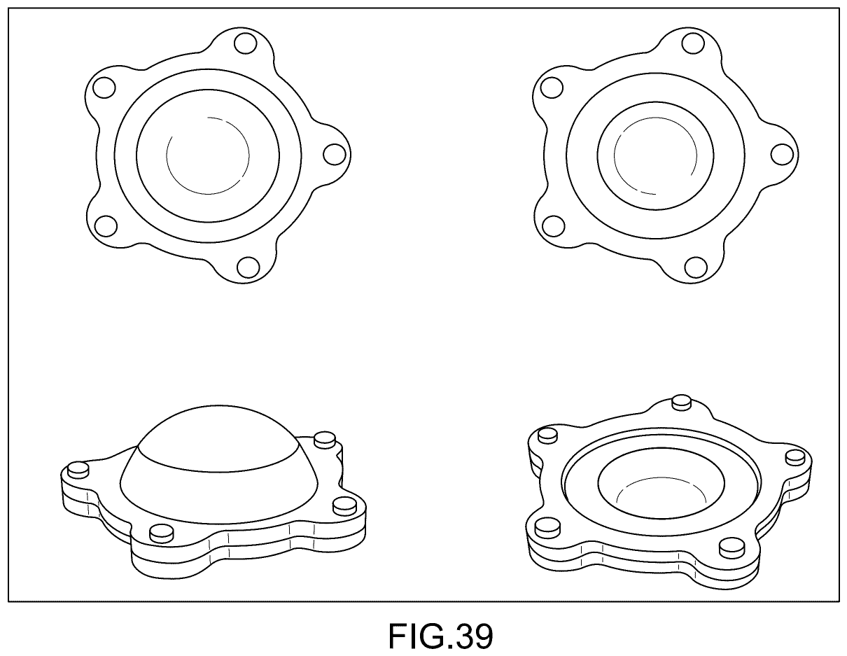

Methods for further processing the substrates of Example 1 include those discussed in U.S. Pat. No. 11,097,527 to W. L. Gore & Associates, which issued on Aug. 24, 2021 and is incorporated herein by reference in its entirety. The substrates may generally be processed according to the teachings to provide stored length and micro-pleats as described herein. FIG. 39 illustrates components that may be implemented in accordance with the methods described in U.S. Pat. No. 11,097,527 for processing the implantable substrate 12 to form the material 10 from the implantable substrate 12. Referring to FIGS. 7-9, the method may include positioning the implantable substrate 12 between two sheets of material, removing air, and setting the implantable substrate in the shape (e.g., inducing the crystalline state). In some embodiments, the method may further include positioning the implantable substrate 12 with a stretched elastomeric material during the processing of the implantable substrate 12 and then releasing the elastomeric material to a non-stretched configuration, wherein the elastomeric material facilitates the formation or definition of the pleats 16 and folds 18 in the implantable substrate 12. Once the pleats 16 and folds 18 are defined, the processes described in U.S. Pat. No. 11,097,527 may be implemented to process the implantable substrate 12. Regions of differing stored length may be implemented by stretching the elastomeric material more or less across portions of the elastomeric material.

### Non-Woven Implantable Substrates

Various examples and features of non-woven substrates are provided hereafter. The non-woven substrates incorporate, at least partially, the features as described previous with respect to the implantable substrate 12.

### Example 3

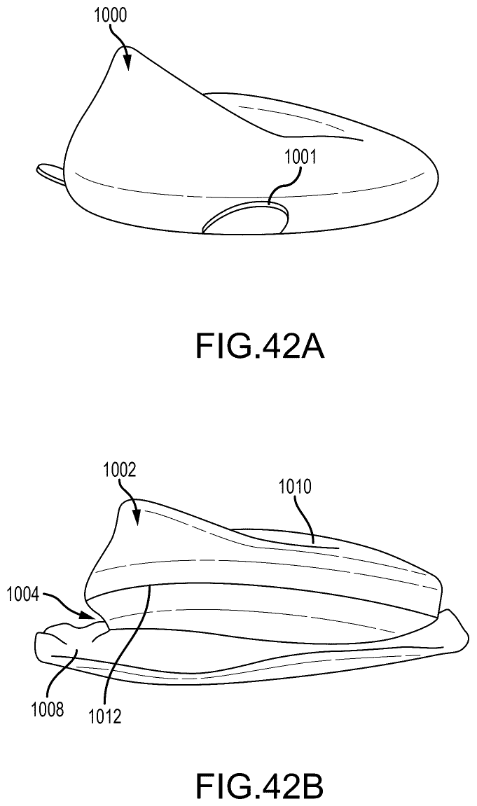

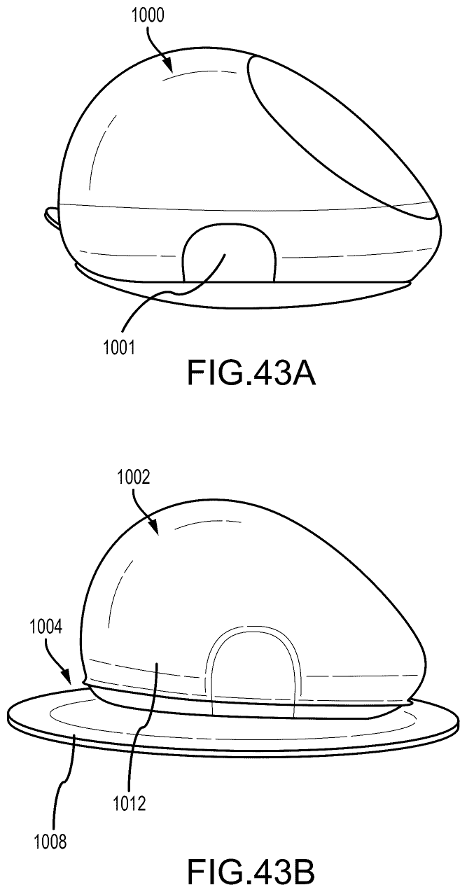

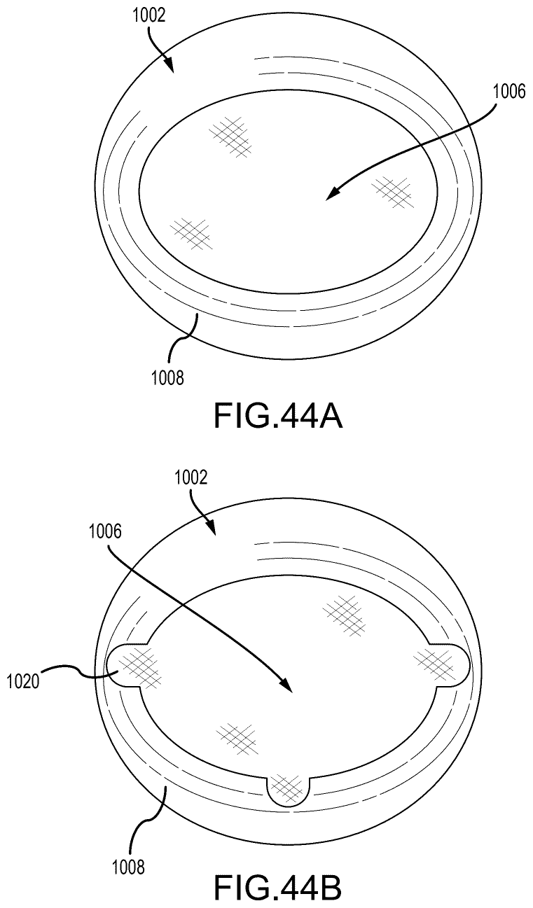

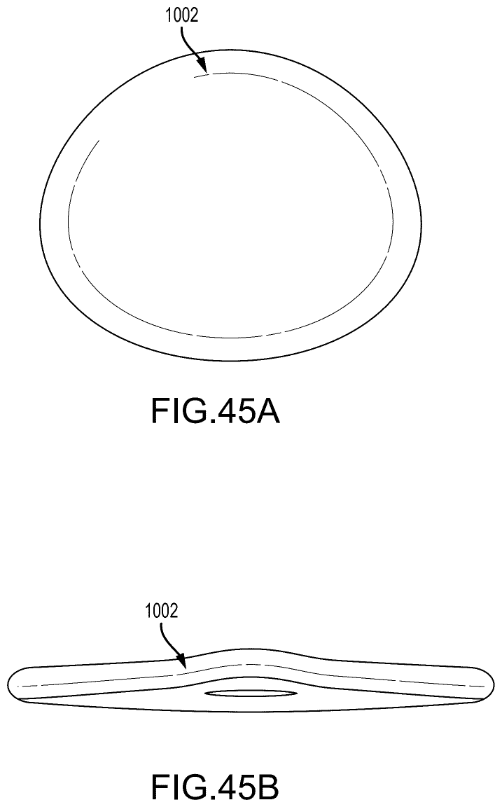

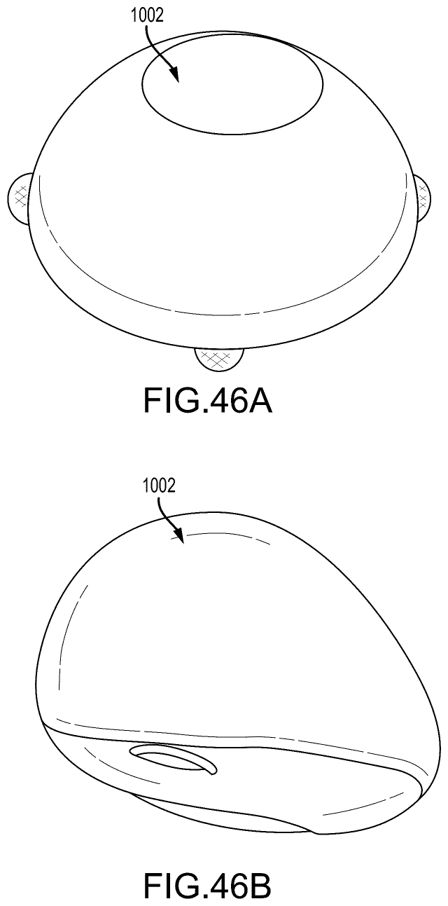

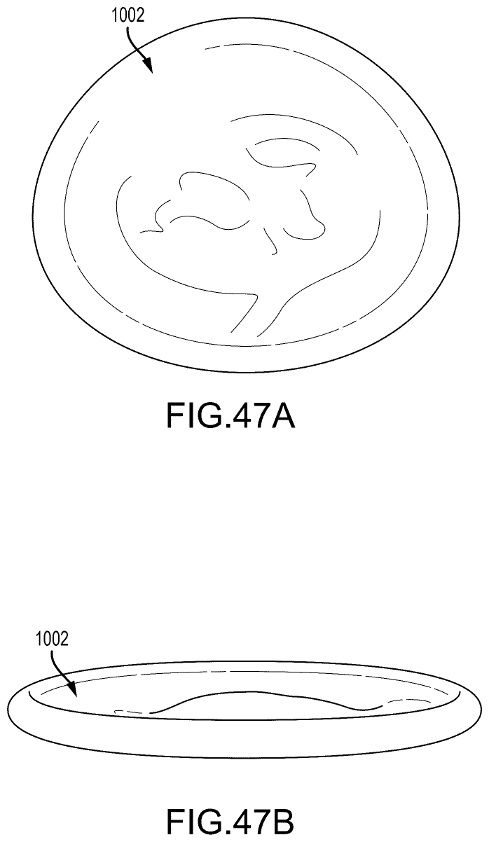

Exemplary embodiments of a tissue expander 1000 and an implantable substrate 1002 are provided in FIGS. 42A-47B. The tissue expander 1000 is shown being partially filled in FIG. 42A and fully filled in FIG. 43A. The implantable substrate 1002 incorporates the features discussed throughout to provide a three-dimensional substrate that is capable of conforming to the shape of the tissue expander 1000 at various fill levels as demonstrated in FIGS. 42B and 43B. The implantable substrate 1002 is capable of expanding with the tissue expander 1000 as the tissue expander 1000 is filled. Additionally, the implantable substrate 1002 is provided with a narrowed portion 1004 to define a cavity 1006 (See FIGS. 44A and 44B) within which the tissue expander 1000 is positioned. The implantable substrate 1002 further includes a flange 1008. The narrowed portion 1004 is designed to facilitate capture and retention of the tissue expander 1000 within the cavity. The implantable substrate 1002 includes relatively more stretch in a center portion 1010 to accommodate the tissue expander profile and expansion profile as compared to the stretch of an outer circumference portion 1012. The flange 1008 includes little-to-no stretch to facilitate support and fixation. Each of the narrowed portion 1004, flange 1008, center portion 1010, and circumference portion 1012 are formed of a single piece of material. It is understood that materials (e.g., pieces of material with varying amounts of stored length) may be combined (e.g., via welding or adhesives) to define the various portions (e.g., to form flanges or posterior sections). FIGS. 44A and 44B illustrate the narrowed portion 1004 and the flange 1008 which accommodates the tissue expander at a low fill volume (FIG. 44A) and a high fill volume (FIG. 44B). Anchoring tabs 1001 of the tissue expander 1000 may be pulled out of the implantable substrate 1002 (e.g., through slits). FIGS. 45A-47B demonstrate an implantable substrate 1002 before, during and after expansion of the tissue expander (not shown). As shown in FIGS. 47A and 47B, the implantable substrate 1002 recovers the stored length that was released during expansion (see FIGS. 46A and 46B) and substantially returns to its original configuration without creases or wrinkles.

### Example 4

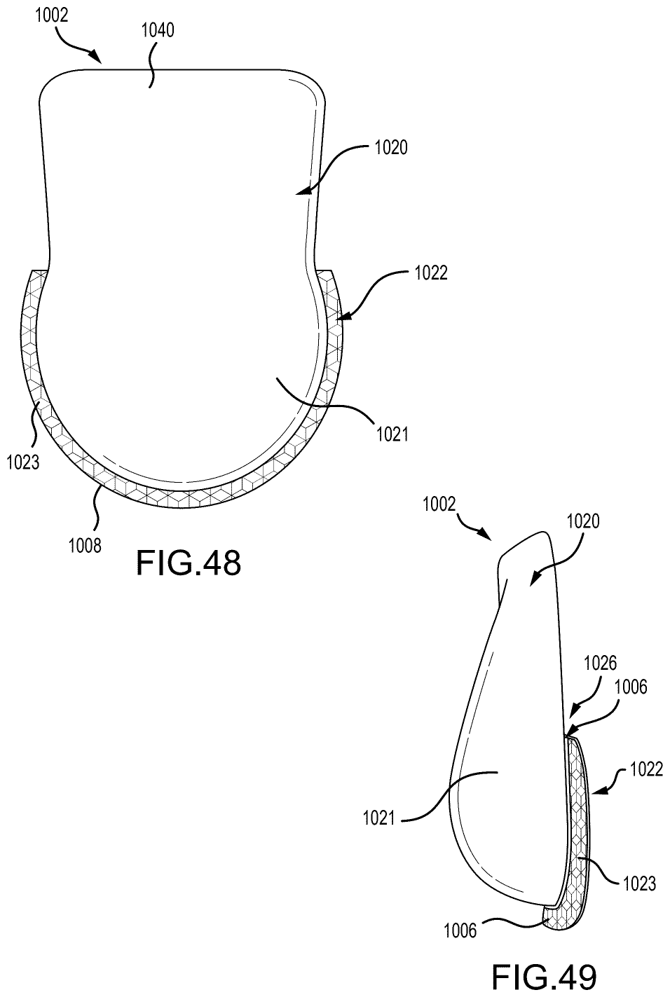

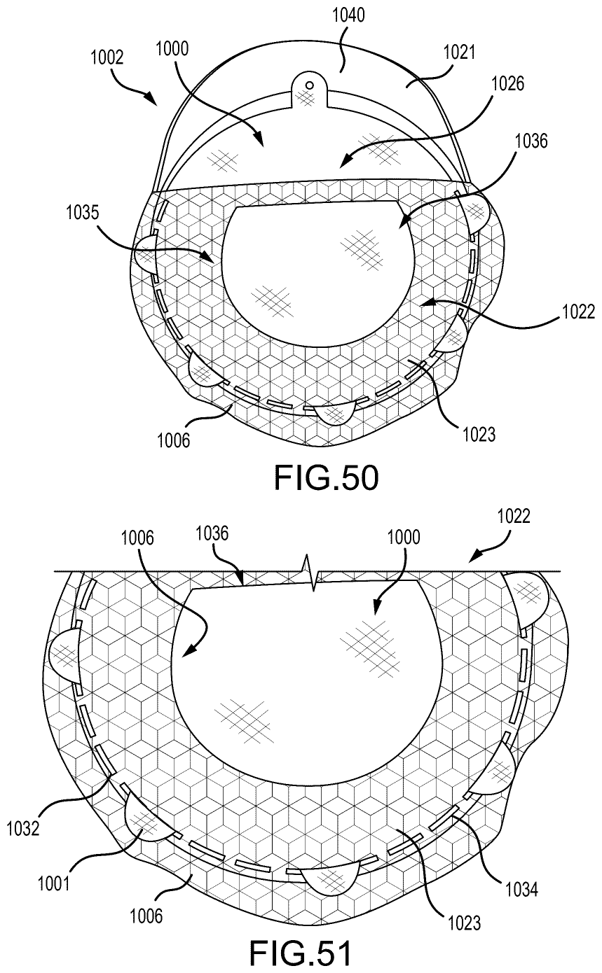

Referring to FIGS. 48 and 49, another embodiment of an implantable substrate 1002 and an implantable device (tissue expander 1000) (positioned inside the implantable substrate, see FIGS. 50-51) is illustrated. Similar to previously discussed embodiments, the implantable substrate 1002 includes at least one bioabsorbable polymer defining a tissue scaffold. For example, the bioabsorbable polymer may include those materials previously discussed and may be provided as a sheet or have a bulk three-dimensional structure. The implantable substrate 1002 may include an anterior side 1020 and a posterior side 1022 defining a cavity 1006 therebetween, where the cavity 1006 is configured to accommodate the tissue expander 1000. The anterior side 1020 may be a first tissue scaffold 1021 and the posterior side 1022 may be a second tissue scaffold 1023. The first and second tissue scaffolds 1021, 1023 are discussed hereafter in further detail. Furthermore, although tissue expanders are discussed throughout this and other examples provided herein, it is understood that other implantable devices may be implemented other than tissue expanders. For example, a permanent implant may be implemented instead of a tissue expander or subsequent to the use of a tissue expander.

In some embodiments, at least a portion of the implantable substrate 1002 includes recoverable stored length and at least a portion of the implantable substrate 1002 is inelastic. For example, the posterior side 1022 may include a portion of the implantable substrate 1002 that is inelastic and the anterior side 1020 may include a portion of the implantable substrate 1002 that includes recoverable stored length. It is understood that the entirety of the posterior side 1022 does not have to be inelastic or that the entirety of the anterior side 1020 does not have to include recoverable stored length. More specifically, in some embodiments, the first tissue scaffold 1021 may be provided with portions that include recoverable stored length as previously discussed herein (for example, substrates formed to include pleats or micro pleats such that the bulk property of the substrates demonstrate elasticity or recoil toward a relaxed configuration) and the second tissue scaffold 1023 may be provided with portions that do not include stored length (e.g., the bulk property of the scaffold does not demonstrate elasticity).

In some embodiments the anterior and posterior sides 1020, 1022 are formed of a single substrate. In some embodiments, the anterior and posterior sides 1020, 1022 are formed of two independent substrates. For example, the anterior side 1020 may include a first tissue scaffold 1021 that is coupled to a second tissue scaffold 1023 that defines the posterior side 1022. The first and second tissue scaffolds 1021, 1023 may be coupled by various methods and means, including but not limited to sonic welding, thermal welding, an adhesive, bonding, or suturing. The first and second tissue scaffolds 1021, 1023 are coupled to each other around only a portion of the periphery of each of the first and second tissue scaffolds 1021, 1023 such that an opening 1026 is provided into the cavity 1006 formed between the first and second tissue scaffolds 1021, 1023. In some embodiments, the first and second tissue scaffolds 1021, 1023 are coupled to each other from around about 25% of the periphery to about 85% of the periphery of the second tissue scaffold 1023 (see also FIG. 50). For example, the first and second tissue scaffolds are coupled to each other from about 25% of the periphery to about 50% of the periphery of the second tissue scaffold 1023, from about 50% of the periphery to about 70% of the periphery of the second tissue scaffold 1023, or from about 70% of the periphery to about 85% of the periphery of the second tissue scaffold 1023.

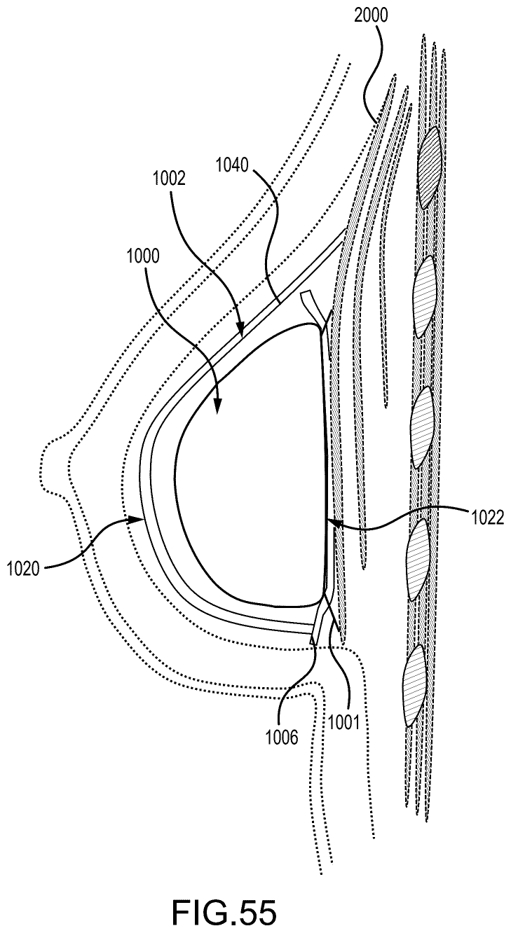

Referring to FIGS. 50 and 51, in some embodiments, the posterior side 1022 may include a flange 1008. The flange 1008 may be defined on the second tissue scaffold 1023 or may be coupled to the second tissue scaffold 1023. In some embodiments, the flange 1008 may extend partially or fully around the periphery of the posterior side 1022. In some embodiments, the flange 1008 is operable to extend beyond the anchoring tabs 1001 of the tissue expander 1000 (see also FIG. 55) when the tissue expander 1000 is disposed within the cavity 1006. This facilitates coverage or isolation of the anchoring tabs 1001 relative to at least one side of the tissue proximate the implantable medical device. In embodiments in which the anchoring tabs 1001 are positioned through at least a portion of the second tissue scaffold 1023 (e.g., respective slits 1032), the anchoring tabs 1001 may be separated or isolated from the tissue (e.g., via the flange 1008) anterior to the anchoring tabs 1001 while the anchoring tabs 1001 are coupled to the tissue posterior to the anchoring tabs 1001. It is understood that in some embodiments, the flange 1008 may be shorter than the anchoring tabs 1001 and are not fully covered. It is also understood that in some embodiments, a flange may not be implemented and the anchoring tabs 1001 are not covered.

In accordance with an embodiment, the anchoring tabs 1001 extend through the second tissue scaffold 1023, wherein the second tissue scaffold 1023 may include slits 1032 through which the anchoring tabs 1001 may extend. The slits 1032 may be provided corresponding to the size of the anchoring tabs 1001, or various slits 1032 may be provided that are separated by tags 1034, the slits 1032 being expandable by disabling (e.g., cutting, tearing, etc.) the tags 1034 between corresponding slits 1032. In this way, the slits can be customized during a procedure for the specific implantable device (e.g., various tissue expanders 1000) and orientation of the implantable device that is being implemented. The anchoring tabs 1001 are understood to provide support the implantable device (e.g., tissue expander 1000, permanent implant, etc.) itself and/or may add to the structural support of the flange 1008, the implantable substrate 1002, and/or the patient's native tissue to support of the weight of the implantable device when implanted. Upon implantation, the anchoring tabs 1001 may be coupled (e.g., sutured) to the patient's tissue, in accordance with an embodiment. However, it is understood that various arrangements may be implemented. For example, in some embodiments, the anchoring tabs 1001 may be coupled to the flange 1008 or otherwise to the implantable substrate 1002 and the implantable device (e.g., the tissue expander 1000 or a permanent implant) is supported relative to the patient by the implantable substrate 1002 or the combination of the flange 1008, the implantable substrates 1002, and the patient's native tissue. In some embodiments, the anchoring tabs 1001 may extend through the slits 1032 but are not coupled to the patient's tissue, such that the implantable substrate 1002 supports the implantable device (e.g., the tissue expander 1000 or a permanent implant). In some embodiments, the implantable device (e.g., the tissue expander 1000 or a permanent implant) is supported by the implantable substrate 1002 (for example, in the cavity 1006). In some embodiments, the flange 1008 and the anchor tabs are both coupled (e.g., sutured) to the patient's tissue. It is understood that these various configurations may be implemented and combinations thereof, either for specific scenarios or by preference of the surgeon.

In some embodiments, the posterior side 1022 further defines an aperture 1036. The aperture 1036 is bounded on its periphery by the second tissue scaffold 1023. The aperture 1036 may be provided for several reasons, including, but not limited to, reducing the mass and volume of the implantable substrate 1002, access to the cavity 1006, free fluid flow, and so forth. The aperture 1036 may be provided in any number of shapes, including those shown such as a partial circle with a chord extending across a portion of the periphery, ovular, and so forth. In some embodiments, the aperture 1036 may be bounded by a cinch portion 1035. The cinch portion 1035 may include recoverable stored length. The cinch portion 1035 may help ensure that the implantable device (e.g., tissue expander 1000, permanent implant, etc.) is retained in the cavity 1006. For a tissue expander 1000, the cinch portion also facilitates retention even when the tissue expander 1000 is being expanded to a greater volume. The cinch portion 1035 may be provided as an integral portion of the second tissue scaffold 1023 (e.g., treated as discussed herein to include recoverable stored length) or may be coupled to the second tissue scaffold 1023 (e.g., an elastic component such as a band coupled to the second tissue scaffold 1023).

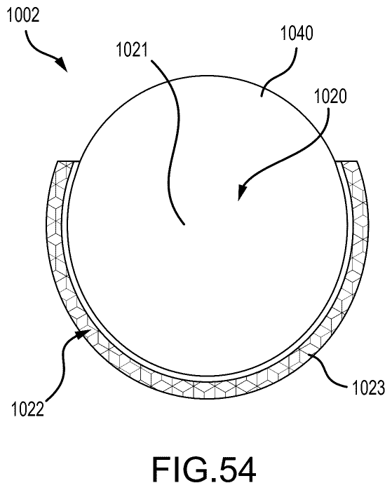

Turning now to a more specific discussion of the anterior side 1020, the implantable substrate 1002 may include an extension portion 1040 that is defined as a portion of the first tissue scaffold 1021. The extension portion 1040 extends beyond a boundary 1041 of the posterior side 1022 (see FIG. 53) adjacent to the opening 1026. Stated otherwise, the extension portion 1040 extends beyond the outer perimeter of the second tissue scaffold 1023. More specifically, the first tissue scaffold 1021 may include a first length and the second tissue scaffold 1023 may include a second length, where the first length is greater than the second length. The lower end of each of the first and the second tissue scaffolds 1021, 1023 may be positioned adjacent each other such that the upper end of the first tissue scaffold 1021 overhangs the second tissue scaffold 1023. The extension portion 1040 may be provided in a variety of shapes. For example, FIG. 48 illustrates an extension portion 1040 with a squared/rectangled profile with rounded corners. Other embodiments may include a rounded or semicircular profile (See FIGS. 53 and 54). The extension portion 1040 does not necessarily need to be provided as having a symmetrical profile, but may be provided with an asymmetrical profile. In some embodiments, the extension portion 1040 may be provided oversized such that a physician can modify (e.g., cut) the extension portion 1040 to the specific anatomy and desired result (e.g., upper pole shape). It is noted that any profile that is relevant to the anatomy into which the implantable substrate 1002 is configured to be implanted, including but not limited to rounded, square, or cut in a way to easily be folded in on itself.

When implanted, the extension portion 1040 can be coupled to the patient's tissue. In some embodiments, the extension portion 1040 is coupled (e.g., via sutures, etc.) to the patient's tissue on the same general surface as the anchoring tabs 1001 (e.g., pectoral muscle for tissue expanders or permanent implants in breast reconstruction) above the position of the anchoring tabs 1001. The extension portion 1040 may be coupled at a position such that the anterior side 1020 is substantially taut. As the tissue expander 1000 is filled, the anterior side 1020 is configured to release stored length. FIGS. 49 and 55 illustrate embodiments of the tissue expander 1000 used in conjunction with the implantable substrate 1002 in a breast reconstruction setting. The extension portion 1040 is implemented as discussed above (i.e., coupled to patient tissue above the anchoring tabs 1001) in order to provide a more natural or desired breast shape after reconstruction. More specifically, the extension portion 1040 allows for a more natural looking upper pole of the breast (see FIG. 55). The extension portion 1040 provides a smoother transition from the breast to the upper chest to limit the formation of a dimple or undercut (e.g., avoids the “rock-in-a-sock” or “half-orange” breast shape after reconstruction). In some embodiments, the extension portion 1040 defines a first length along a first longitudinal axis this is disposed in a first direction and the flange 1008 defines a second length along a second longitudinal axis that is disposed in the first direction, wherein the first length is greater than the second length. However, it is understood that in some embodiments, the second length may be greater than the first length such that the flange 1008 extends beyond the extension portion 1040.

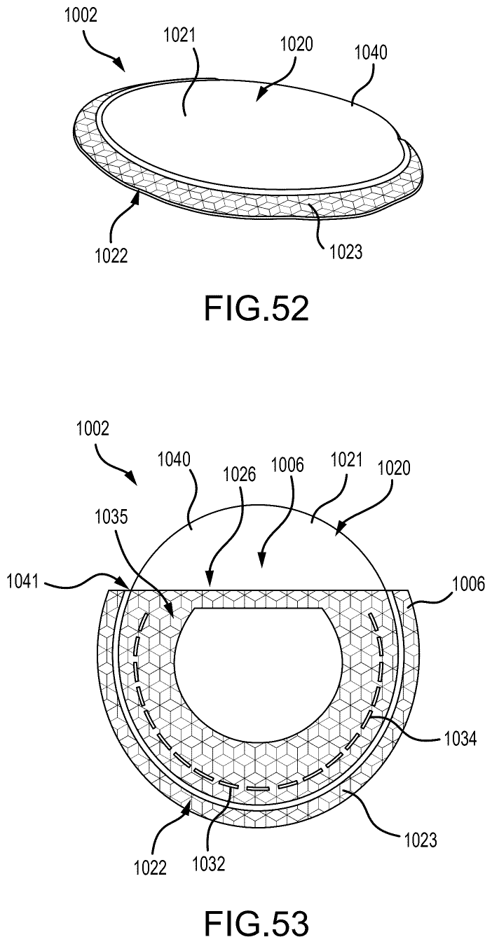

Referring to FIGS. 52-54, the implantable substrate 1002 may be provided as a substantially flat construct. The first tissue scaffold 1021 and the second tissue scaffold 1023 may be provided as flat sheets that are coupled to each other. The first and second tissue scaffolds 1021, 1023 may be coupled via sonic welding, thermal welding, an adhesive, bonding, suturing, or other fastening mechanism. Various advantages may be realized by a flat construct, including ease of manufacturing and packaging of flat assemblies.

Referring to FIG. 55, an illustration of an implantable device (e.g., tissue expander 1000) and implantable substrate 1002 are shown implanted in a patient, in accordance with an embodiment. It is understood that the implantable device may include various devices besides tissue expanders, such as permanent implants and so forth. The anchoring tabs 1001 are coupled to the patient's tissue (e.g., pectoral muscle 2000) to support the tissue expander 1000 during expansion of the tissue (although other coupling arrangements are contemplated and set forth herein). Various positions of implantation are contemplated including subpectoral implantation. The tissue expander 1000 is positioned in the cavity 1006 of the implantable substrate 1002 such that the tissue expander 1000 substantially supports the implantable substrate 1002 (at least until tissue ingrowth into the implantable substrate 1002 occurs). In some embodiments, tissue integrates into the implantable substrate 1002 and the implantable substrate 1002 is resorbed. In such embodiments, the tissue incorporation allows the tissue to develop and ultimately carry the load of the implantable device (e.g., a permanent implant). The extension portion 1040 is coupled to the patient's tissue above the tissue expander 1000 to provide a slope simulating a natural upper pole of the breast. The flange 1008 is positioned covering or overlapping the anchoring tabs 1001 in order to limit contact or rubbing against tissue to which the anchoring tabs 1001 are not secured. In some embodiments, the flange 1008 may be secured to the tissue (e.g., sutured to the pectoral muscle).

A method of reconstructing a breast may include positioning an implantable substrate 1002 in a target position proximate a pectoral muscle in a patient, the implantable substrate 1002 including a bioabsorbable polymer that is inelastic defining a tissue scaffold including a posterior side 1022 and an anterior side 1020, the posterior and anterior side 1022, 1020 defining a cavity 1006 therebetween, the anterior side 1020 including an extension portion 1040 that includes recoverable stored length, and positioning an implantable device (e.g., a tissue expander 1000 or a permanent implant) in the cavity 1006 of the tissue scaffold.

In embodiments in which a tissue expander 1000 is implemented, the method may include expanding the implantable medical device to a larger volume. The implantable substrate 1002 and/or the implantable medical device may be coupled to tissue of the patient as previously discussed. The extension portion 1040 may also be coupled to the tissue of the patient at a location that provides a natural contour to the breast as it relates to the upper pole shape.

Further embodiments and features of the implantable substrate are provided hereafter. It is understood that any of the previously discussed features or features that are discussed hereafter may be incorporated to any of the designs discussed herein to the extent that they are geometrically feasible, and thus none of the features is to be considered limited to any of the specific embodiments of a single figure or portion of the description. Accordingly, any of the features of Examples 1-4 may be implemented with respect to any of the other examples provided, and any of the features discussed with respect to any embodiments not specifically labelled as an “Example” may be implemented with respect to any of the Examples or embodiments discussed herein.

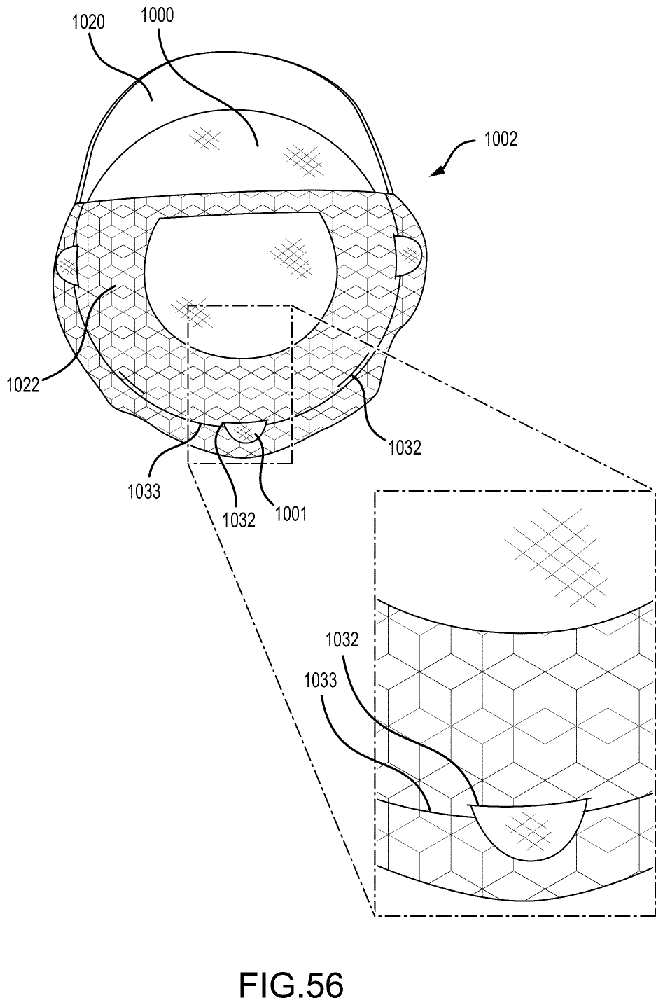

Referring now to FIG. 56, in some embodiments, the slits 1032 are provided adjacent to a coupling position 1033 between the posterior and anterior sides 1022, 1020. For example, in some embodiments, the slits 1032 are spaced from the coupling position 1033 by about 0.5 inches or less (e.g., less than 0.4 inches, less than 0.3 inches, less than 0.2 inches, or less than 0.1 inches). The relatively small distance between the slits 1032 and the coupling position 1033 facilitates the ability to suture the tissue expander 1000 and/or the implantable substrate 1002 to each other or the supporting tissue. In some embodiments, the coupling position 1033 represents a weld line between the anterior and the posterior sides 1020, 1022 (although other means of coupling are contemplated including sutures and so forth, as well as the coupling position representing a transition from anterior to posterior sides 1020, 1022 for devices made of a single substrate). The slits 1032 may be provided with a width that is substantially equal to or greater than the width of the anchoring tabs 1001 (see FIG. 56-58). This may be in contrast to the slits 1032 of FIG. 53 which may be provided with tags 1034 therebetween which can be cut by physicians for sizing the slits 1032 to accommodate the anchoring tabs 1001. The slits 1032 can be provided at various increments to accommodate various anchoring tabs 1001 and tissue expanders 1000 generally. For example, the slits 1032 may be provided at 45 degree increments about at least a portion of the implantable substrate 1002. Other spacing is contemplated, including, but not limited to 60 degrees, 30 degrees, 15 degrees, and 10 degrees.

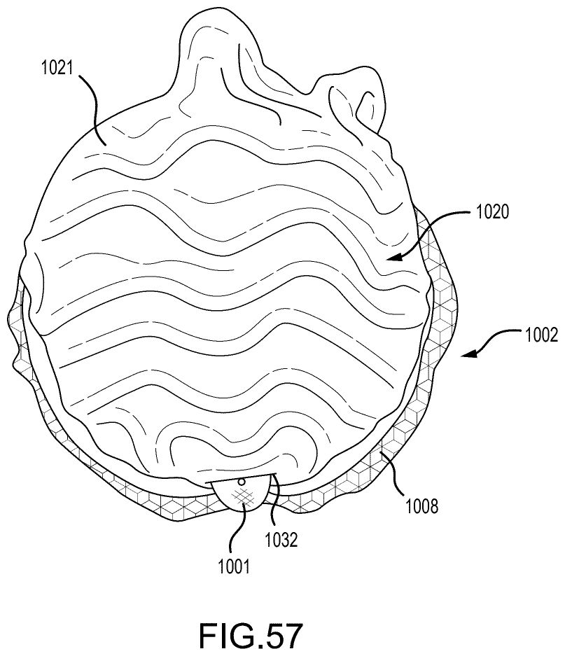

Referring to FIG. 57, in some embodiments, the slits 1032 may be provided on the anterior side 1020 of the implantable substrate. In some embodiments, this places the slits 1032 on material that includes stored length. This, in some embodiments, allows for coupling the anchoring tabs 1001 to tissue on the anterior side 1020 of the device. Additionally, this may facilitate the ability to suture through the anchoring tabs 1001 of the tissue expander 1000 and through the flange 1008 of the implantable substrate 1002.

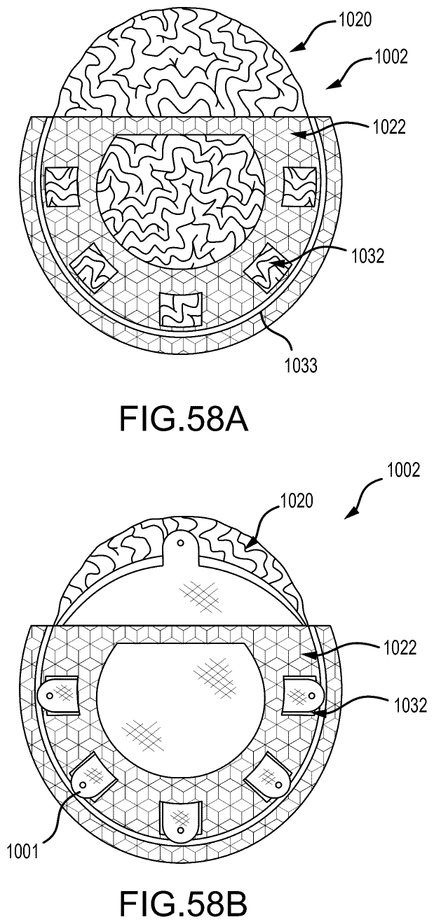

Referring to FIGS. 58A and 58B, in some embodiments, the slits 1032 may be provided as windows that have a length that is at least 50% of the width of the slits 1032. This allows for greater visibility and access to anchoring tabs 1001 during use. For example, when the tissue expander 1000 is positioned in the implantable substrate 1002 and being implanted into the patient, the slits 1032 being configured as windows with the additional length allows the physician to more readily assess the alignment of the anchoring tabs 1001 with the slits 1032 and provides easy access to the anchoring tabs 1001. As previously indicated, the slits 1032 may be provided on either the posterior or the anterior side 1022, 1020.

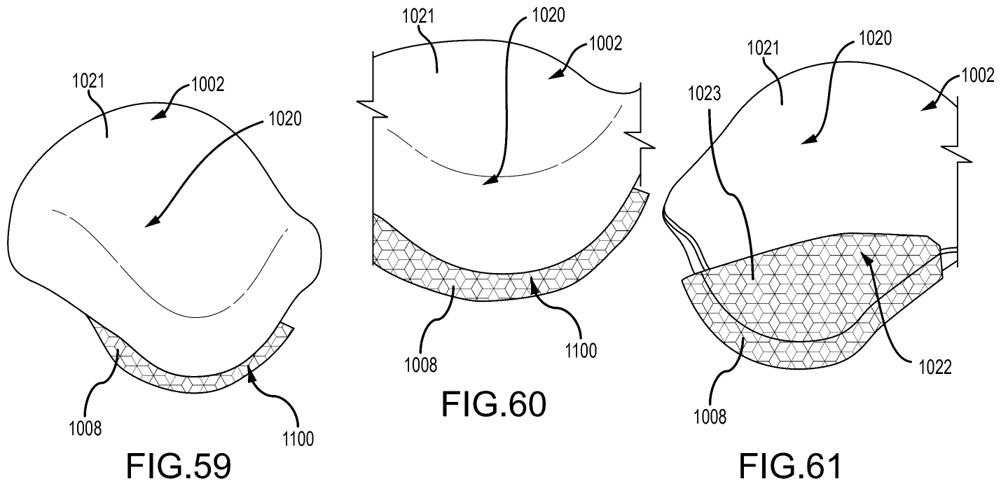

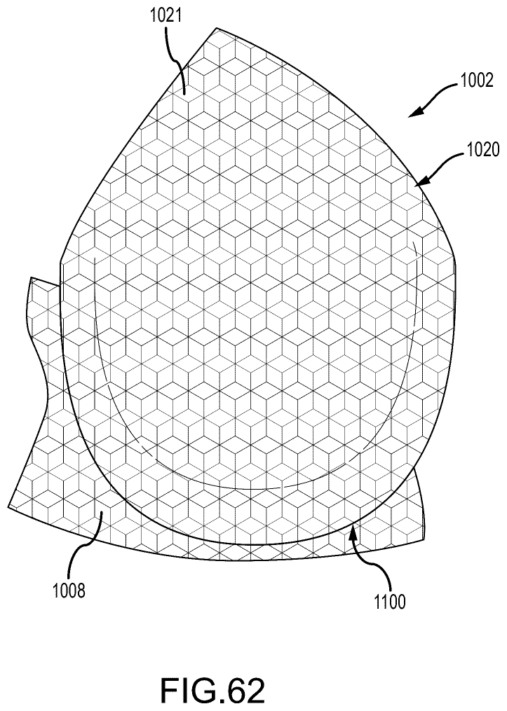

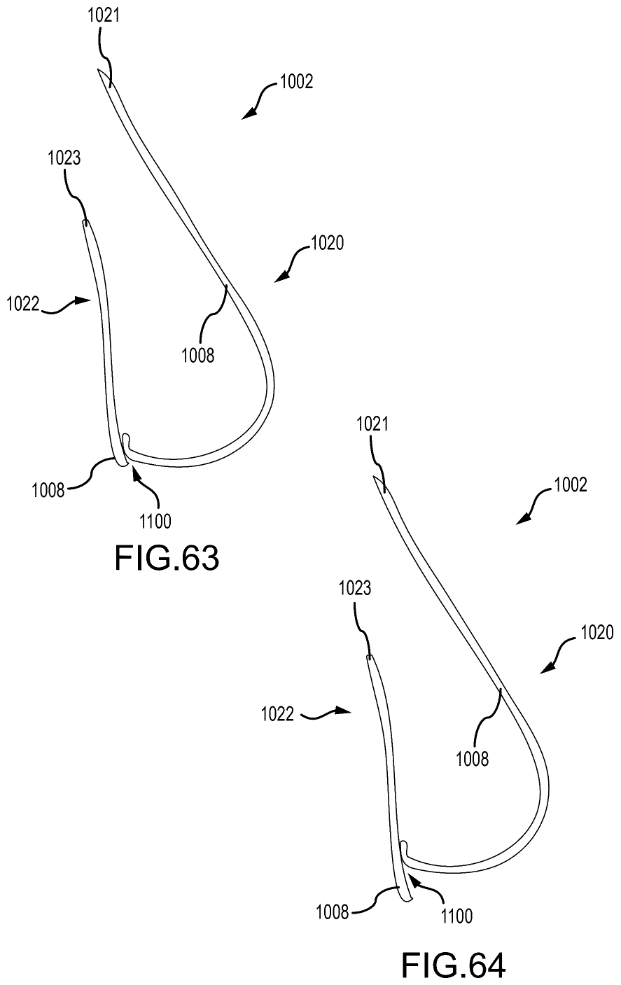

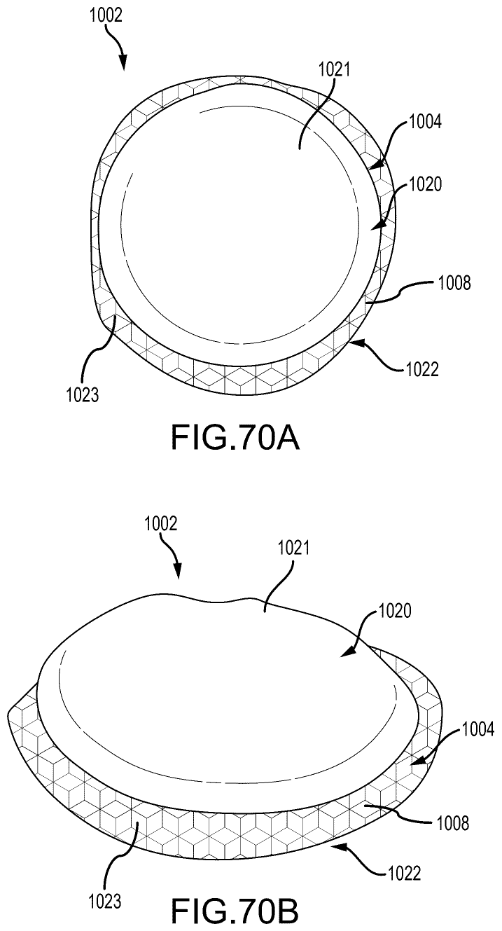

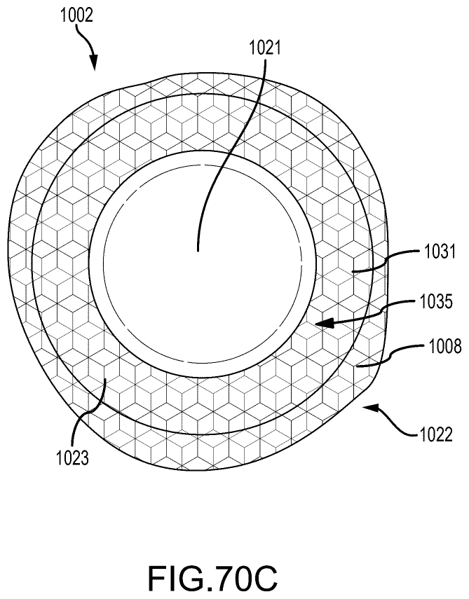

Referring to FIGS. 59-62, in some embodiments the implantable substrate 1002 is provided with a fold 1100 that mimics the inframammary fold of natural breast tissue. For example, in some embodiments, the fold 1100 is provided in a device with anterior and posterior sides 1020, 1022 implementing a first and second tissue scaffold 1021, 1023 by placing the coupling position 1033 or weld on the posterior side 1022 of the implantable substrate 1002. For example, FIG. 61 illustrates a weld along the second tissue scaffold (on the posterior side 1022 of the implantable substrate 1002) which defines the flange 1008 beyond the weld. The weld is provided from about a 5 o'clock position to about a 7 o'clock position to mimic the inframammary fold but could be provided over a smaller or larger arc (e.g., from about 3 o'clock to about 8 o'clock). FIGS. 59, 60, and 62 illustrate the anterior side 1020 which shows the fold 1100 on the first tissue scaffold 1021 mimicking the inframammary fold. Because the fold 1100 is provided on the first tissue scaffold 1021 at least a portion of the flange 1008 may be concealed. FIGS. 63 and 64 provide cross sections of the implantable substrate 1002 in which a fold 1100 is shown and all or a portion of the flange 1008 is concealed by the anterior side 1020. In some embodiments, the flange 1008 is continuous about a periphery of the implantable substrate 1002, in some embodiments, the flange 1008 extends partially about the periphery of the implantable substrate 1002, and in some embodiments, the flange 1008 extend intermittently about the periphery of the implantable substrate 1002. In some embodiments, the size of the flange 1008 is variable along the periphery of the implantable substrate 1002. In some embodiments, the flange 1008 is narrower at a lower position corresponding to a lower pole of a breast. In some embodiments, the flange 1008 is narrower at a 6 o'clock position relative to the flange at a 4-5 o'clock position and a 7-8 o'clock position. The flange 1008 may include a variable width at different positions about the periphery of the implantable substrate 1002. Referring to FIG. 70A for example, the flange 1008 extend further at a lower side of the implantable substrate 1002 as compared to an upper side of the implantable substrate 1002. In some embodiments, the flange 1008 covers or at least partially covers the anchoring tabs 1001. In some embodiments, the flange 1008 does not cover the anchoring tabs 1001.

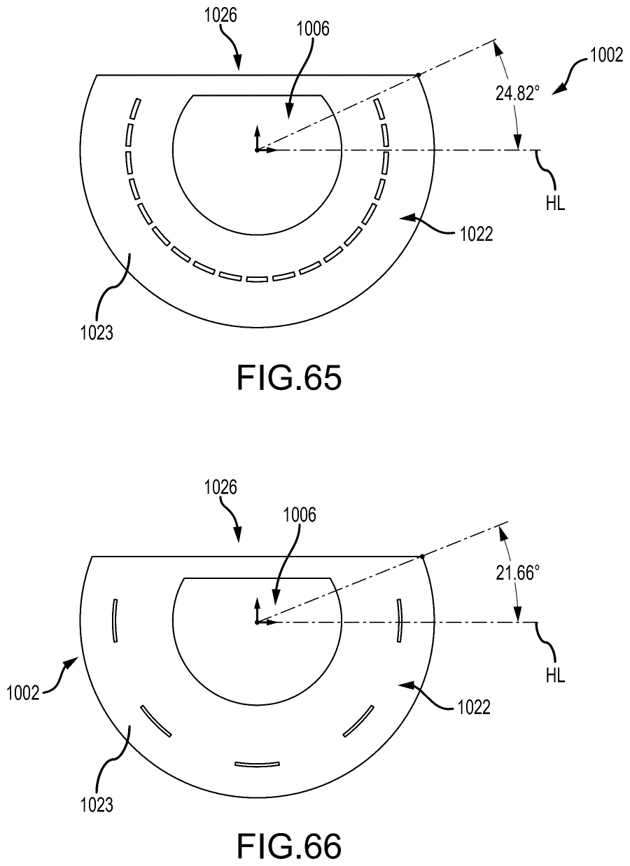

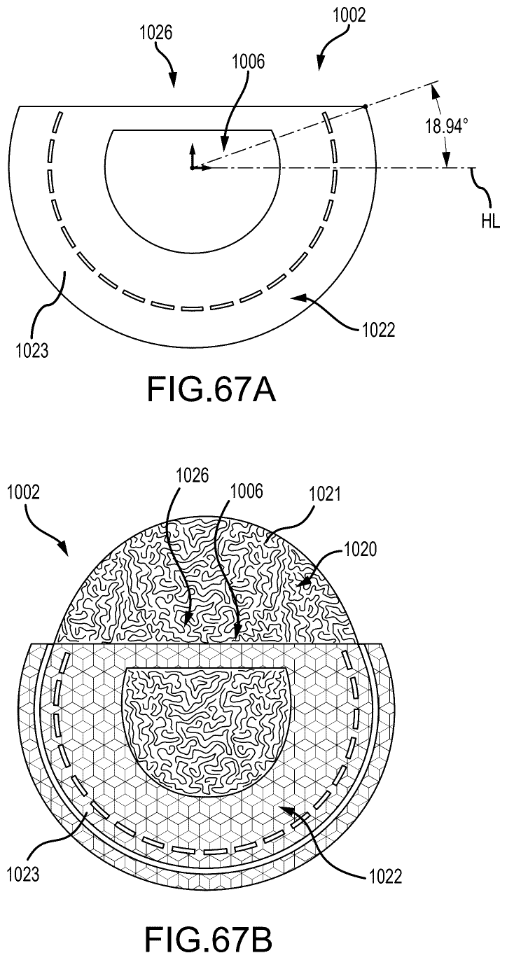

Referring to FIGS. 65-67B, in some embodiments, the implantable substrate 1002 may be provided with a posterior side 1022 that provides an opening 1026 into the cavity 1006 (see FIG. 50) that is wide to provide easy access into the cavity 1006 (e.g., for insertion of the tissue expander 1000 or permanent implant). In some embodiments that have a generally arcuate shape on a lower portion of the implantable substrate 1002 or the posterior side 1022, the outer periphery of the second tissue scaffold 1023 on the posterior side 1022 defined by the arc may extend less than about 25 degrees above a horizontal line HL defined by the center of the arc. The horizontal line HL may also be defined as extending between the widest portion of the posterior side 1022. For example, the outer periphery of the second tissue scaffold 1023 defined by the arc extends about 19, 21, 23, or 25 degrees above the horizontal line HL defined by the center of the arc. Stated otherwise, the second tissue scaffold 1023 of the implantable substrate 1002 may define a total arc extending about 218, 222, 226, or 230 degrees. By only extending a short degree above the horizontal line HL, the opening 1026 represents about 130, 134, 138, or 142 degrees. The opening 1026 is wide in order to accommodate insertion of devices into the cavity 1006. In some embodiments, the opening 1026 represents less than 180 degrees of the posterior side 122 but greater than 120 degrees. By having the opening 1026 less than 180 degrees of the posterior side 122, the implantable substrate 1002 is capable of retaining the tissue expander 1000 (or permanent implant) with stability (e.g., the width of the opening 1026 is less than an overall width of the tissue expander 1000 (or permanent implant). FIG. 65 illustrates a second tissue scaffold 1023 (or posterior side 1022 design for embodiments implementing a single tissue scaffold) that extends about 25 degrees above the horizontal line defined through the center. FIG. 66 illustrates a second tissue scaffold 1023 (or posterior side 1022 design for embodiments implementing a single tissue scaffold) that extends about 22 degrees above the horizontal line defined through the center. FIGS. 67A and 67B illustrate a second tissue scaffold 1023 (or posterior side 1022 design for embodiments implementing a single tissue scaffold) that extends about 19 degrees above the horizontal line defined through the center, wherein FIG. 67B shows the anterior side 1020 of the implantable substrate 1002.

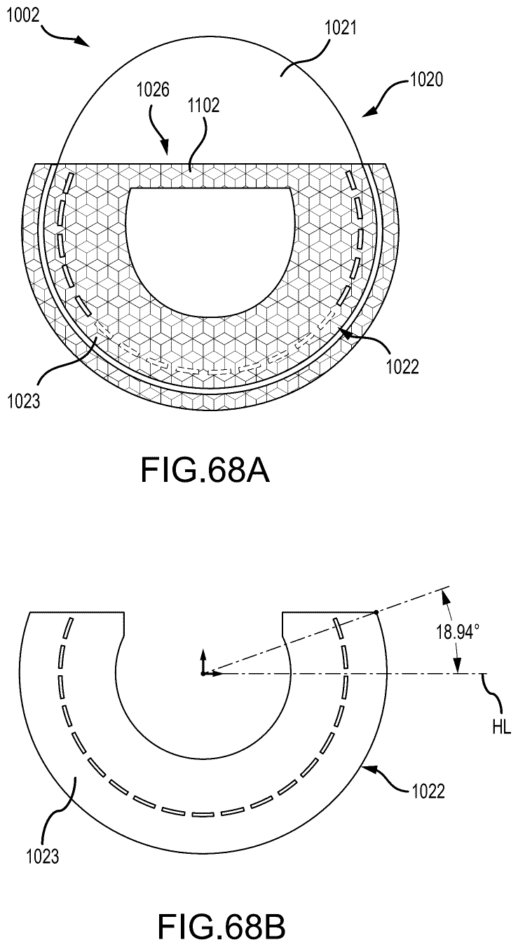

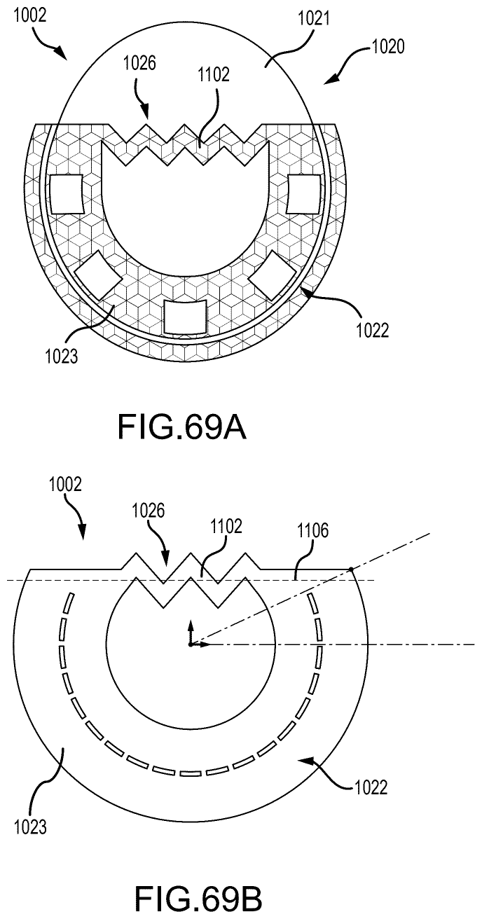

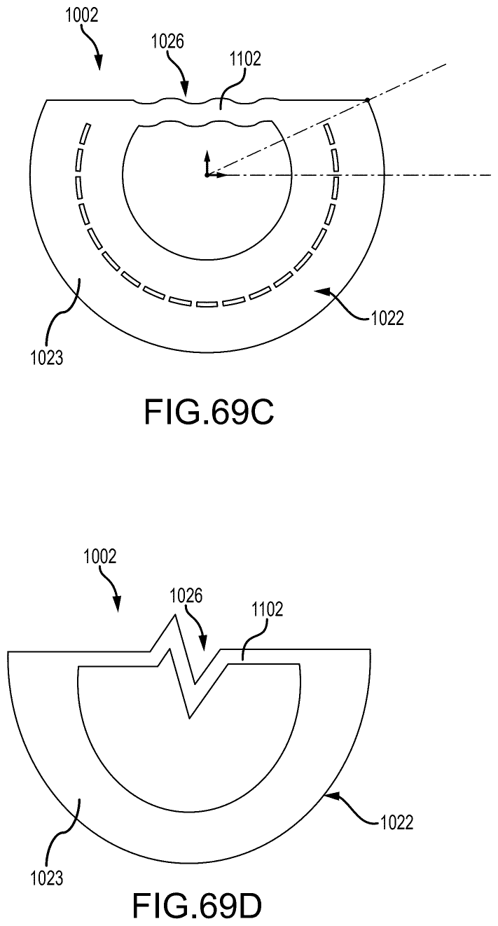

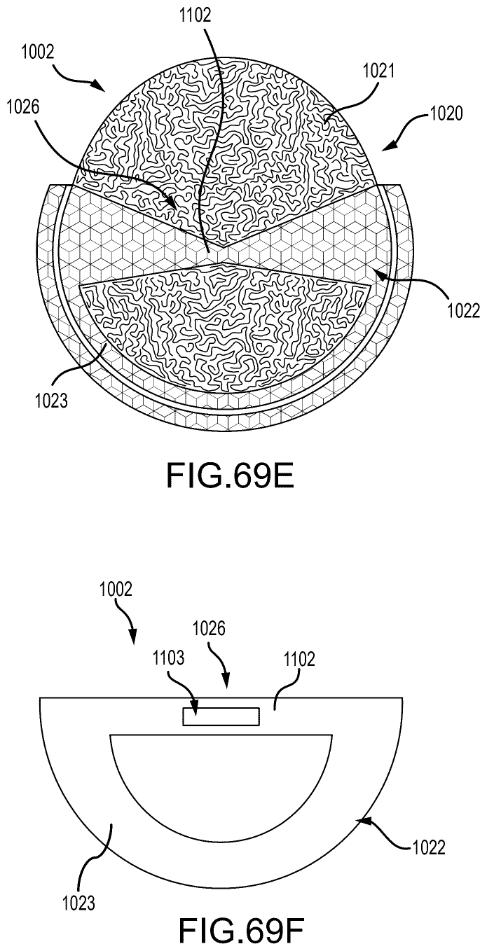

Referring to FIG. 68A, in some embodiments, the second tissue scaffold 1023 that is positioned on the posterior side 1022 may be provided with a material that includes stored length. This allows the opening 1026 to expand (at least temporarily) for insertion or for accommodation of the tissue expander 1000 (or permanent implant). The stored length may be included in the second tissue scaffold 1023, the stored length being provided as previously described with respect to the other components of the implantable substrate 1002. In some embodiments, the second tissue scaffold 1023 includes a bar 1102 that extends across at least a portion of a width of the posterior side 1022 and holds the lateral sides of the implantable substrate 1002 at a predetermined width. However, it is understood that the second tissue scaffold 1023 may not include the bar as shown in FIG. 68B. In some embodiments, the bar 1102 may include various shapes that are configured to elongate when tension is applied across the bar 1102. For example, FIGS. 69A-69F illustrates embodiments in which the bar 1102 includes a zig-zag or sinusoidal shape. As tension is applied across the bar 1102, portions of the bar 1102 are capable of extending or rotating out of plane to facilitate elongation of the bar 1102 which provides a larger opening 1026 through which the tissue expander 1000 (or permanent implant) may be inserted. Various configurations may be provided as seen in FIGS. 69A-69D, included various amplitudes and shapes. For example, in FIG. 69B, the inner apices 1104 overlap a horizontal line 1106 defining a centerline of the bar 1102, whereas the inner apices 1104 of FIG. 69A do not overlap a centerline of the bar 1102. FIG. 69C illustrates a bar 1102 incorporating an undulating or sinusoidal shape. FIG. 69D illustrates a bar 1102 incorporating a single zig-zag 1105 with the remainder of the bar 1102 being substantially linear. FIG. 69E illustrates a bar 1102 that has a variable height across the length of the bar 1102. For example, toward the center of the bar 1102, the height tapers to a smaller height from each lateral side. FIG. 69F includes a bar 1102 with an aperture 1103 defined therethrough. It is noted that FIGS. 69A and 69E illustrate implantable substrates 1002 with the second tissue scaffold 1023 coupled to the first tissue scaffold 1021, whereas FIGS. 69B-69D and 69F illustrate only the second tissue scaffold 1023, which would then be used in combination with the first tissue scaffold 1021. It is understood that the design of the second tissue scaffolds 1023 illustrated herein may also be implemented with respect to an implantable substrate 1002 that uses a single tissue scaffold with a posterior side 1022 implementing the designs shown and described with respect to any of the figures included herein.

Referring to FIGS. 70A-70C, in some embodiments, an implantable substrate 1002 is provided with a seam between the first and second tissue scaffolds 1021, 1023 of the anterior and the posterior sides 1020, 1022. The seam may be provided in various manners, including but not limited to sonic welding, thermal welding, an adhesive, bonding, suturing, and a fastening mechanism. In FIG. 70C, the seam is provided by welding and the weld line is included on the posterior side 1022. However, it is understood that the seam (e.g., weld line) may be positioned on the anterior side 1020. The seam may be placed on various sides for various reasons, including but not limited to manufacturability, tissue incorporation, contact with tissue, and so forth. As can be seen in FIGS. 70A-70C and 59-62, the first and second tissue scaffolds 1021, 1023 can be coupled in such a way to provide both the flange 1008 and a cuff 1031, where the cuff 1031 is configured to retain the tissue expander 1000 (or permanent implant). The cuff 1031 may be modified as described herein to act as a cinch portion 1035 for further retaining the tissue expander 1000 (or permanent implant). The coupling may also provide the inframammary fold as previously discussed.

Referring further to FIGS. 70A-70C, in some embodiments the first and second tissue scaffolds 1021, 1023 may be coupled to each other to define a cavity 1006 (similar to the configuration shown and described with respect to FIGS. 44A and 44B) within which the tissue expander 1000 (or permanent implant) is positioned. This arrangement provides a narrowed portion 1004 is designed to facilitate capture and retention of the tissue expander 1000 within the cavity 1006 and a flange 1008 as previously described. This embodiment may be used to preload the tissue expander 1000 (or permanent implant) into or with the implantable substrate 1002, which allows physicians the ability to not have to manipulate the tissue expander 1000 (or permanent implant) with respect to the implantable substrate 1002 when either component is positioned in the patient. In some embodiments, the flange 1008 may be formed of the first tissue scaffold 1021 (e.g., is an extension of the first tissue scaffold 1021).

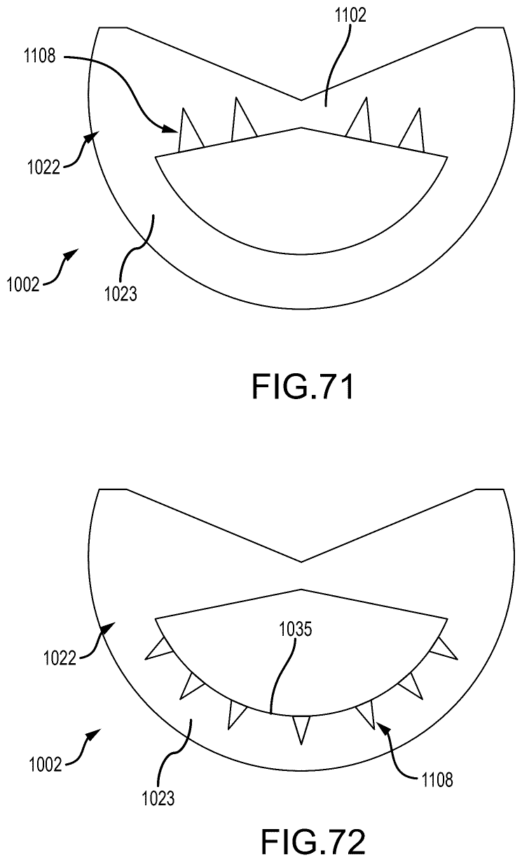

Referring to FIGS. 71 and 72, in some embodiments, the implantable substrate 1002 includes orientation features 1108. The orientation features are provided at a predetermined position(s) (e.g., 6 o'clock) in order to provide visual indication of alignment and orientation of the tissue expander 1000 (or permanent implant) within the implantable substrate. Orientation features 1108 include but are not limited to notches (e.g., cutouts), markings, prints, embroidered or embossed features, and so forth. In some embodiments, the orientation features 1108 are provided on the second tissue scaffold 1023 and on the posterior side 1022 such that the orientation feature 1108 is visible during insertion of the tissue expander 1000 (or permanent implant). However, it is understood that the orientation features 1108 may be positioned anywhere visible to the physician during insertion. A corresponding orientation feature (not shown) may be placed on the tissue expander 1000 (or permanent implant) to further provide indication of alignment and orientation of the tissue expander 1000 (or permanent implant) relative to the implantable substrate 1002. Referring specifically to FIG. 71, the orientation feature 1108 may be positioned on the bar 1102, or referring to FIG. 72 the orientation feature 1108 may be positioned on the cinch portion 1035. Additionally, in embodiments implementing orientation features including notches or cutouts, the notches may also further facilitate insertion of a tissue expander or permanent implant into the implantable substrate 1002.

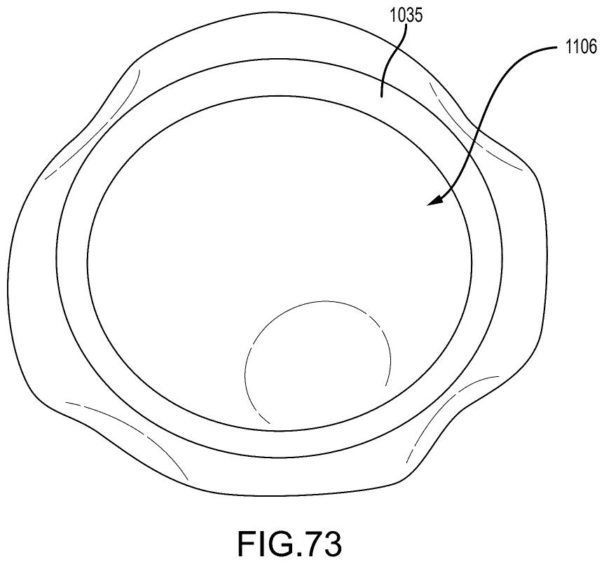

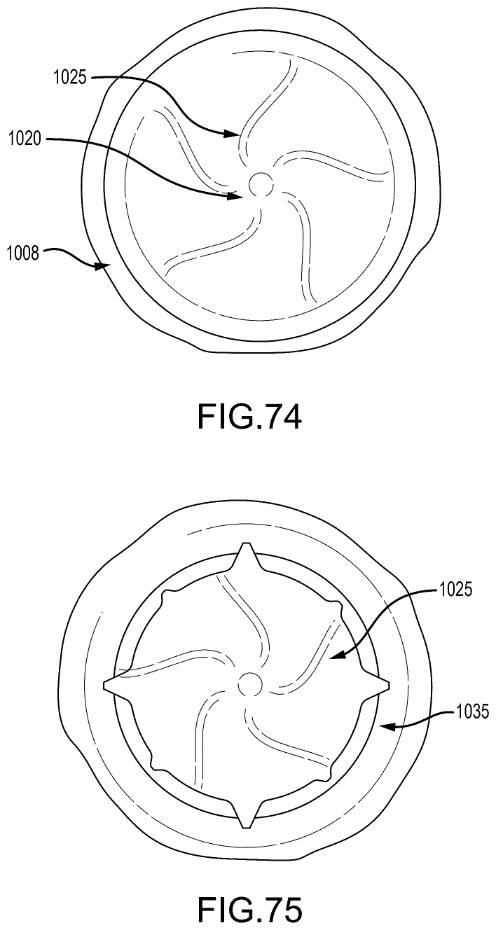

Referring to FIG. 73, in some embodiments the cinch portion 1035 is provided continuously or circumferentially about the opening into the cavity 1006. The cinch portion 1035 provided in this manner is configured to maintain and contain the device within the cavity 1006. Referring to FIG. 75, in some embodiments, the cinch portion 1035 includes notches for accommodating a variety of fill states for a tissue expander 1000 (or various sizes and shapes of tissue expanders or permanent implants). Referring to FIG. 74, in some embodiments the anterior side 1020 is provided with stored length, for example, by including patterned wrinkling 1025 that is provided for accommodating stretch for receiving a tissue expander 1000 (or permanent implant) and includes a flange 1008. The flange 1008 may be provided with as non-elastic substrate without stored length that is capable of facilitating fixation to the tissue expander 1000 (or permanent implant). The cinch portion 1035 may be provided as non-elastic substrate without stored length that secures the tissue expander 1000 (or permanent implant) during perioperative manipulation and/or for quick wrapping of the tissue expander 1000 (or permanent implant).

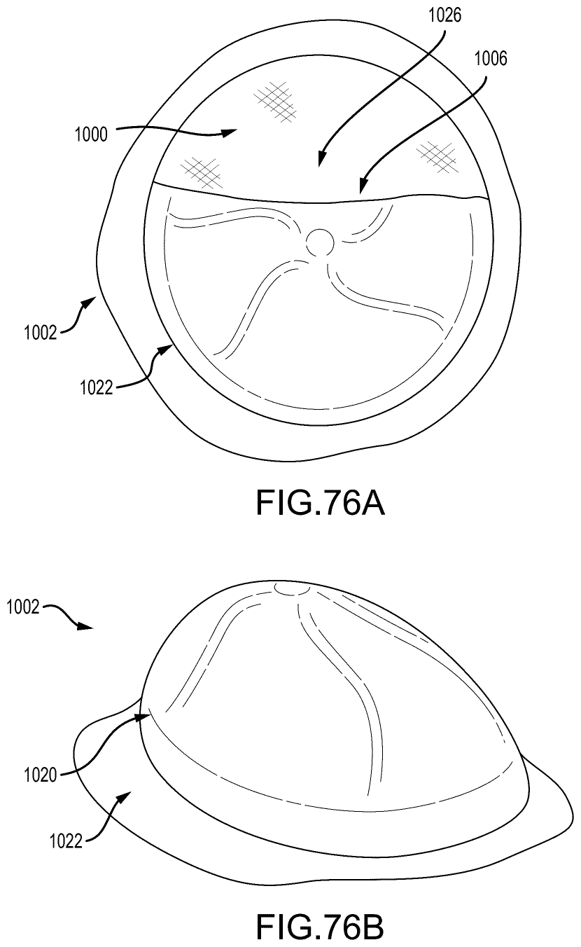

Referring to FIGS. 76A and 76B, in some embodiments, the implantable substrate 1002 includes the anterior side and the posterior side 1020, 1022 (e.g., formed of a single tissue scaffold), where the posterior side 1022 defines the opening 1026 into the cavity 1006 that is off-center. In the embodiment shown in FIG. 76A, the anterior and posterior sides 1020, 1022 are formed of a single substrate and pre-shaped to define an anatomical shape of the breast. The posterior side 1022 is continuous with the anterior side 1020 with the opening 1026 being positioned through the posterior side 1022 and offset toward the periphery of the implantable substrate 1002. For example, the opening 1026 may be offset toward an upper end of the implantable substrate 1002. This allows for the tissue expander 1000 (or permanent implant) to be positioned in the cavity 1006 with the lower portion of the tissue expander 1000 being contained or encapsulated by the implantable substrate 1002, which limits decoupling or shifting of the position of the tissue expander 1000 (or permanent implant) relative to the implantable substrate 1002. The lower portion of the implantable substrate 1002 is thus continuous from the posterior side 1022 to the anterior side 1020. In some embodiments, the lower portion is shaped to include an oblong shape rather than a circular shape in order to better match the anatomical shape of the breast.

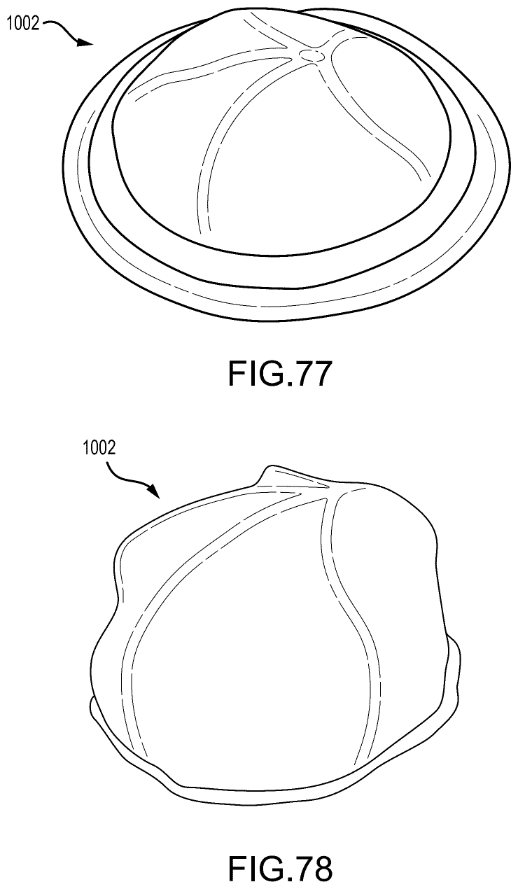

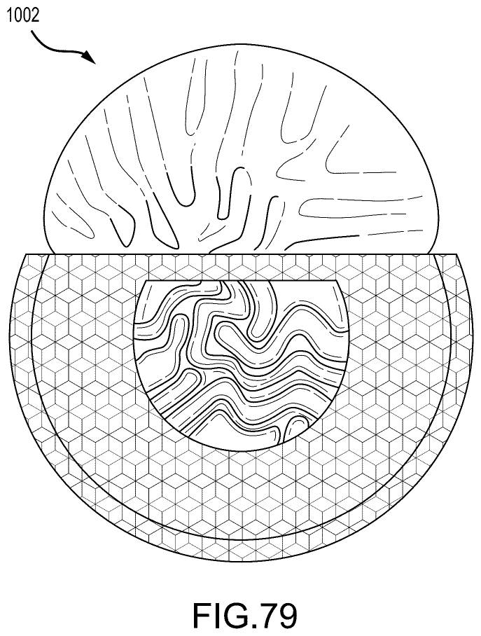

Referring to FIGS. 77-79, in some embodiments, the implantable substrate 1002 may include preferential stretch in predefined regions of the implantable substrate 1002. Preferential stretch may be provided by the methods previously described. For example, regions of the implantable substrate 1002 may be provided with greater stored length than other regions of the implantable substrate 1002. In some examples, preferential stretch may be provided in the lower portion of the implantable substrate 1002. For example, preferential stretch may be provided in the lower portion of the implantable substrate that is positioned at or provides the lower pole of the reconstructed breast. The regions of the implantable substrate 1002 may be provided by including zones or lines of areas of greater or lesser stored length. In some embodiments, an initial shape of the precursor material for forming the implantable substrate 1002 facilitates areas of varying stored length. In some embodiments, this may be accomplished by modifying the implantable substrate 1002 to have areas of lesser stored length by preferentially bonding or fusing zones or lines to limit expansion or stored length as shown in FIG. 77. In some embodiments, this can be accomplished by including patterns with larger and smaller folds, pleats, or microfolds as shown in FIG. 79.

### Knit Implantable Substrates

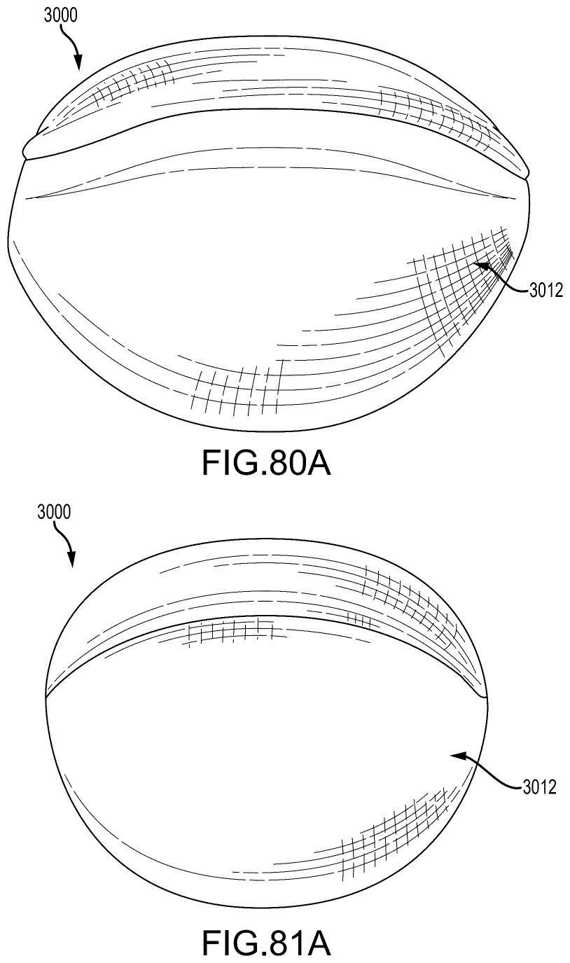

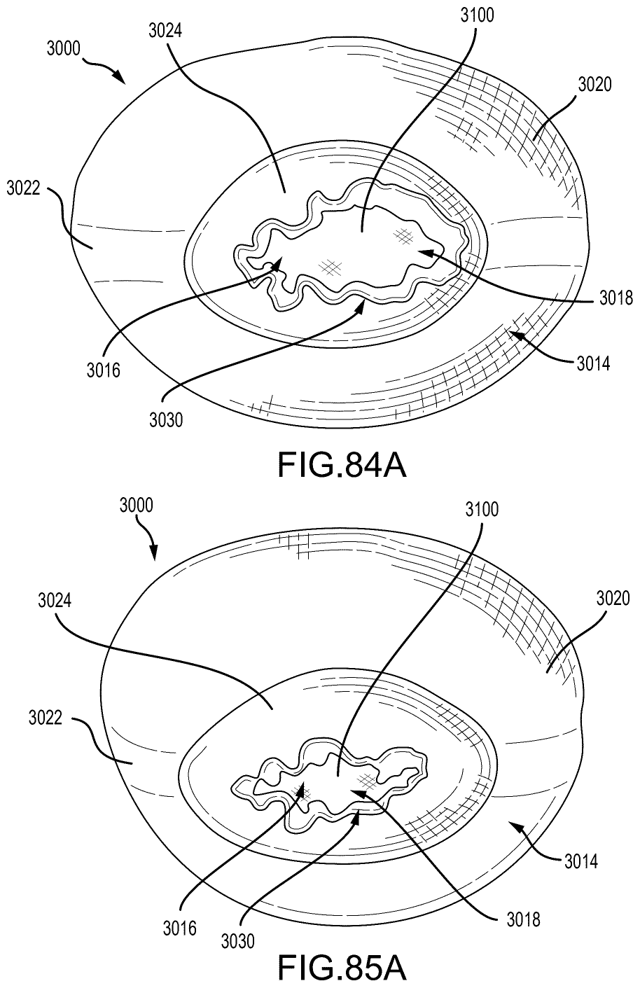

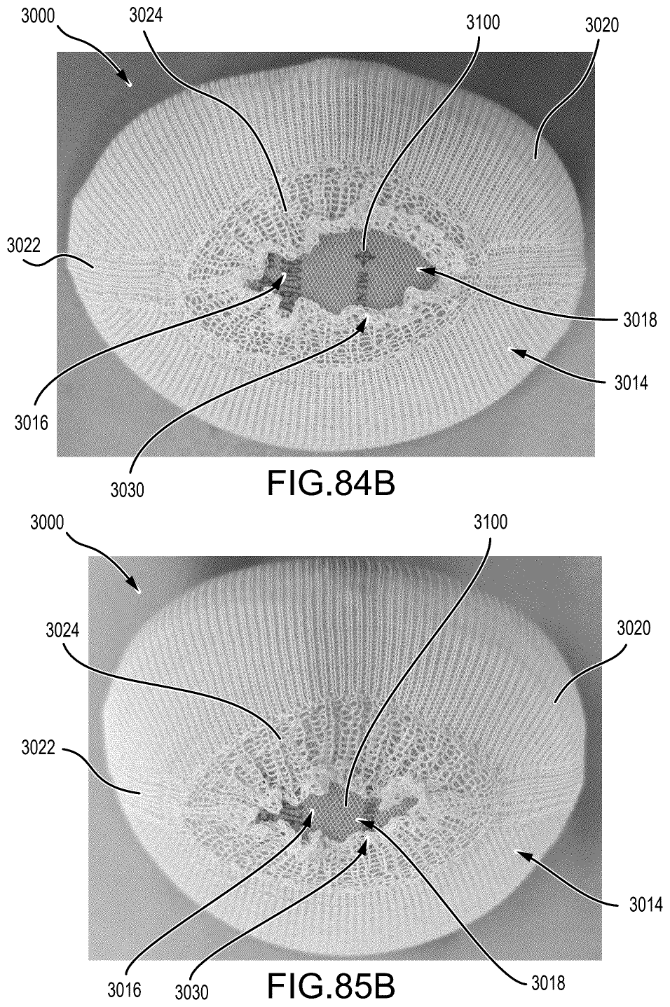

Turning now to a discussion of knit substrates, an implantable substrate 3000 may be provided for implantation. Referring to FIG. 80, an embodiment of an implantable substrate 3000 is illustrated (FIG. 80B being the image thereof), which may be used in various procedures, including but not limited to breast reconstruction. The implantable substrate 3000 may be used with an implantable device 3100 (see FIGS. 84A and 84B) such as a tissue expander or permanent implant. The implantable substrate 3000 is a knit substrate that is bioabsorbable and configured for tissue ingrowth. The implantable substrate 3000 is a knit construct defining a cavity 3016 and an access opening 3018 into the cavity (see FIGS. 84A-85B). The cavity 3016 is configured to receive the implantable device 3100 which is received and removed through the access opening 3018.

In some embodiments, the implantable substrate 3000 includes an anterior side 3012, a posterior side 3014, and the cavity 3016 defined therebetween. The anterior and posterior sides 3012, 3014 may be provided as one integral portion of the same knit construct, or may include a first construct defining the anterior side 3012 and a second construct defining the posterior side 3014 in which the first and second constructs are coupled together (e.g., sewn, bonded, adhered, welded, etc.). The anterior and posterior sides 3012, 3014 are configured to cover a substantial portion of the implantable device 3100 such that the implantable device 3100 is positioned to have minimal contact with surrounding tissue. Instead, the implantable substrate 3000 is configured to be positioned between surrounding tissue and the implantable device 3100 such that the implantable substrate 3000 may facilitate tissue ingrowth into the implantable substrate 3000. The implantable substrate 3000 may provide some support to the implantable device 3100 and can facilitate tissue regeneration for support of the implantable device 3100 (including after the implantable substrate 3000 is at least partially resorbed).

The implantable substrate 3000 further defines the access opening 3018 that is in communication with the cavity 3016. The access opening 3018 facilitates access to the cavity 3016. This, for example, provides access into the cavity 3016 for exchanging implantable devices. In some embodiments, a tissue expander may be implemented for expanding tissue to a specific configuration. After the tissue is expanded, the tissue expander may be removed from the cavity 3016 via the access opening 3018 and a permanent implant may be inserted through the access opening 3018 and positioned in the cavity 3016. In some embodiments, the implantable substrate is resorbed prior to removal of the implantable device from the cavity 3016. This, for example, may be implemented in a breast reconstruction in which the skin and other tissue needs to be expanded over time in order to accommodate a permanent breast implant.

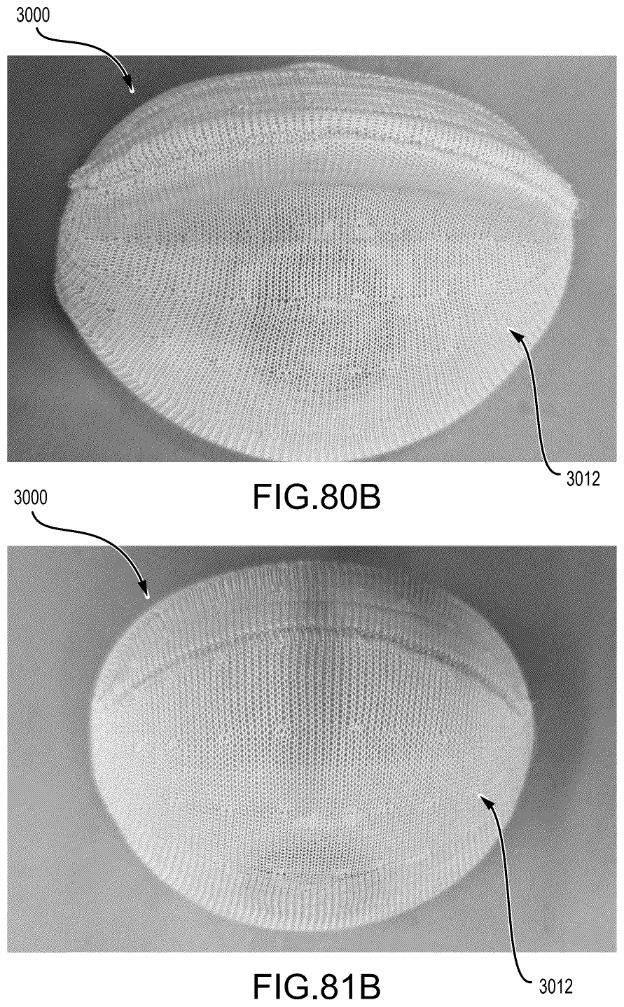

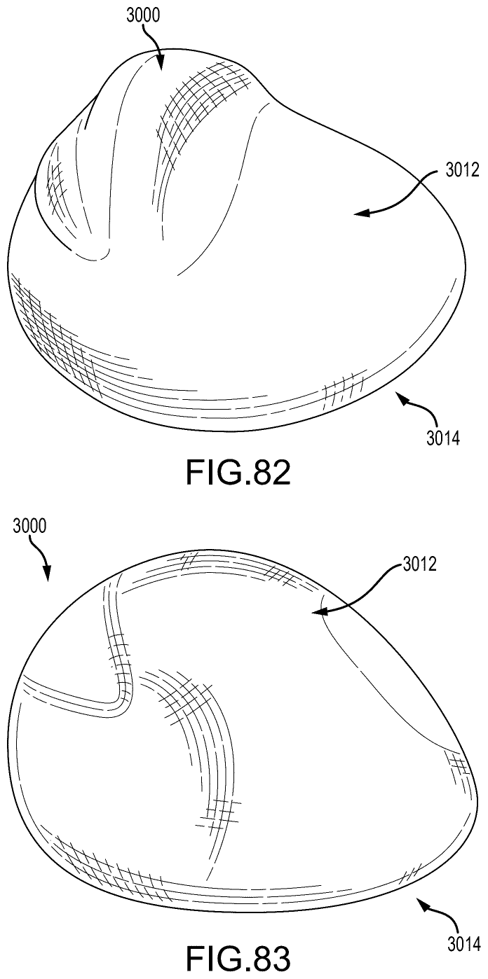

In some embodiments, the implantable substrate 3000 is configured to stretch and/or expand from a first configuration in which the cavity 3016 includes a first volume toward a second configuration in which the cavity 3016 includes a second volume that is greater than the first volume. This allows the implantable substrate 3000 to conform to the implantable device 3100 at various sizes or volumes. Various states of fill of a tissue expander can be seen in FIGS. 80-86. For example, FIGS. 80, 82, and 84 depict the implantable substrate 3000 surrounding a tissue expander (e.g., implantable device 3100), when the tissue expander is in a partially filled state. FIGS. 81, 83, and 85 depict the implantable substrate 3000 surrounding the tissue expander, when the tissue expander is in a filled state. By providing the implantable substrate 3000 that includes stretchability, conformability, expandability, or combinations thereof as part of the implantable substrate's 3000 bulk material properties, the implantable substrate 3000 is capable of adjusting to various sizes of implantable devices, either because of size differences or because of expansion, which facilitates a decreased number of size options necessary for physicians and hospitals to stock. Furthermore, the ability of the implantable substrate 3000 to be able to expand, stretch, and/or conform during implantation reduces the likelihood of undesired folds, bends, wrinkles, overlap, creases, and so forth, of the implantable substrate 3000 during use. Because the implantable substrate 3000 includes stored length and/or has a bulk material property exhibiting stretchability, conformability, and/or expandability, the implantable substrate can be sized to snugly fit the implantable device 3100 (e.g., a tissue expander) without and folds, bends, wrinkles, overlap, creases, and so forth, and can continue to expand (e.g., as the tissue expander is filled). Because the implantable substrate 3000 can stretch to various sizes and configurations, this negates the potential further need to exchange or manually modify the implantable substrate as a tissue expander is filled or as the tissue expander is exchanged for a permanent implant.

In some embodiments, the implantable substrate 3000 may be provided in a three-dimensional configuration (e.g., knitted to have a bulk three-dimensional shape), which is capable of receiving the implantable device 3100. Although specific shapes for the implantable substrate 3000 are shown in the figures, it is understood that various patterns may be implemented for various applications or desired outcomes (e.g., sizes of breast implants, shapes of breast implants, implants in other anatomy, and so forth). The anterior side 3012 may be provided to accommodate the implantable device 3100 that provides a desired shape (e.g., a specific breast shape). The implantable substrate 3000 may be formed in various three-dimensional shapes via a knitting pattern that is dimensioned specifically for the application. Such three-dimensional shapes are provided as the general shape, but it is understood and discussed herein that the implantable substrate 3000 includes a three-dimensional shape that can be generally modified by stretching of the implantable substrate 3000 and conforming (e.g., via drapability, stretching, etc.) of the implantable substrate 3000 to another object (e.g., the implantable device 3100). Stated otherwise, the implantable substrate 3000 is manufactured to include a three-dimensional shape as manufactured and includes a three-dimensional shape regardless of contact with a secondary object or manipulation by the physician. In some embodiments, the implantable substrate 3000 includes a three-dimensional shape as manufactured and includes a three-dimensional shape prior to contact with a secondary object or manipulation by the physician, but is operable to conform itself to the shape of a secondary object or the manipulations of a surgeon via its stretchability, conformability, and/or expandability. In some embodiments, the implantable substrate 3000 includes a three-dimensional shape as manufactured and includes a three-dimensional shape prior to contact with a secondary object or manipulation by the physician, where the three-dimensional shape may support, and in some cases confer some shape to, the implantable device 3100. In such embodiments, the implantable substrate 3000 may be provided with some stretchability, conformability, and/or expandability such that the implantable substrate 3000 and the implantable device 3100 cooperate together to provide the final shape of the system. The three-dimensional shape refers to a gross topography of the implantable substrate 3000 and is not intended to be understood as porous structures, surface textures, individual knits, and so forth.

In some embodiments, the implantable substrate 3000 may be provided as at least two sheets coupled to each other. In some embodiments, the two sheets are substantially flat, meaning having a two-dimensional shape. The two sheets of material can be provided in various shapes and include various mounts of stored length.

The stretchability, conformability, and expandability exhibited by the implantable substrate 3000 in its bulk material properties may be facilitated by the material used in the knit and/or the knit pattern implemented. For example, in some embodiments, the fiber used to form the knit includes elastic properties. In some embodiments, the fiber used to form the knit is inelastic or includes little elasticity (e.g., along the longitudinal axis of the fiber), but the knit pattern imparts a bulk stretchability, conformability, and/or expandability to the implantable substrate 3000 (e.g., stretch along 1 or 2 axes). By knitting the fiber to define the implantable substrate 3000, a larger variety of material may be implemented (e.g., materials that are desirable for certain properties such as bioabsorbability) while still facilitating the implantable substrate 3000 having a bulk stretchability, conformability, and/or expandability. It is understood that stretchability, conformability, and/or expandability of the implantable substrate 3000 relates to the expandability of the implantable substrate 3000 as a whole facilitated by the knit pattern, the relative positions and lengths of fiber between knits, and relative movement of the fiber in relation to the knits (e.g., fiber to fiber contact or the fiber to fiber interaction). For example, the knits allow the fiber(s) to move relative to itself/each other at each knit, which allows the implantable substrate 3000 to exhibit a bulk stretchability, conformability, and/or expandability as the slip, slide, and/or adjust at various knit positions.

The implantable substrate 3000 may be provided including a single knit pattern or a combination of knit patterns. In some embodiments, regions of the implantable substrate 3000 may be defined by a first knit pattern and a second knit pattern. Each knit pattern can provide various amounts of stretch and/or axis of stretch. Thus, in some embodiments, one or more portions or regions of the implantable substrate 3000 may include greater stretchability than one or more other portions of the implantable substrate. For example, the anterior side 3012 of the implantable substrate 3000 may include a first knit pattern and the posterior side 3014 of the implantable substrate 3000 may include a second knit pattern that is different from the first pattern. The first pattern may include a pattern that exhibits relatively more stretch than the second pattern. In this manner the anterior side 3012 can stretch or expand relatively more than the posterior side 3014 to accommodate the implantable device 3100 (e.g., expansion of a tissue expander). For example, if a tissue expander is being implemented, the posterior side 3014 of the implantable substrate 3000 may expand less or have less stretchability, conformability, and/or expandability relative to the anterior side 3012 in order to facilitate a stable portion (e.g., less expansion) for tissue incorporation on the posterior side (e.g., proximate the pectoral muscle, which is not expanded in a pre-pectoral breast reconstruction) and allows the anterior side 3012 to expand with the tissue (e.g., fascia, fat, skin, etc.) that is expanded by a tissue expander as it is filled. This example is not to be read is limiting and it is understood that more than two regions of the implantable substrate 3000 may be provided with various knit patterns in order to facilitate specific functionalities, and the regions are not meant to be understood as only applicable to either the anterior or posterior sides 3012, 3014 as a whole. Instead, each of the anterior and posterior sides 3012, 3014 may include one or more regions of knit patterns.

Referring to FIGS. 84 and 85, the posterior side 3014 of the implantable substrate 3000 is illustrated. In some embodiments, the posterior side 3014 includes at least two different knit patterns. For example, the posterior side 3014 includes a first region 3020 including a first knit pattern, a second region 3022 including a second knit pattern, and a third region 3024 with a third knit pattern. The first region 3020 may be bisected by the second and third regions 3022, 3024 where the second region 3022 is positioned extending substantially linearly, and the third region 3024 is positioned substantially surrounding the access opening 3018. The third region 3024 may include a different knit pattern in order to facilitate insertion and removal of the implantable device 3100 into and out of the cavity 3016 through the access opening 3018. Thus, in some embodiments, the knit pattern of the third region 3024 may be different in that it exhibits greater expansion or stretch than the knit patterns of the first and second regions 3020, 3022.

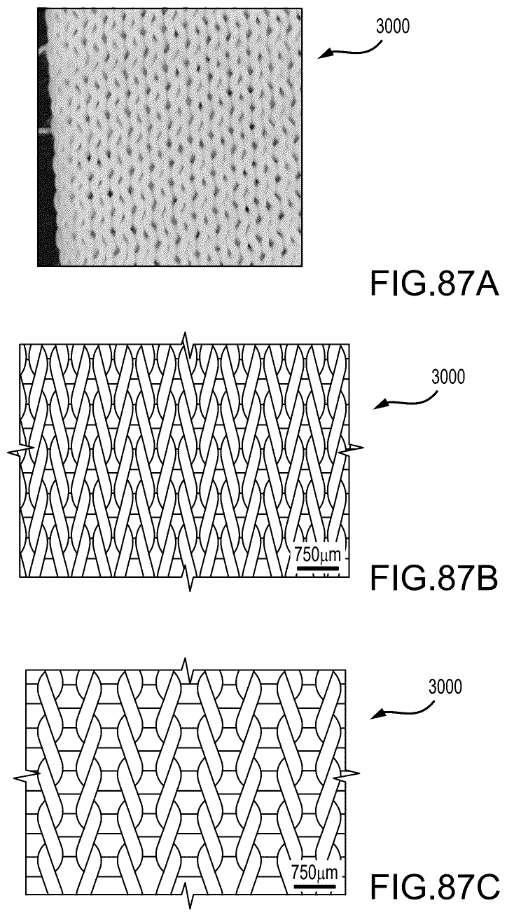

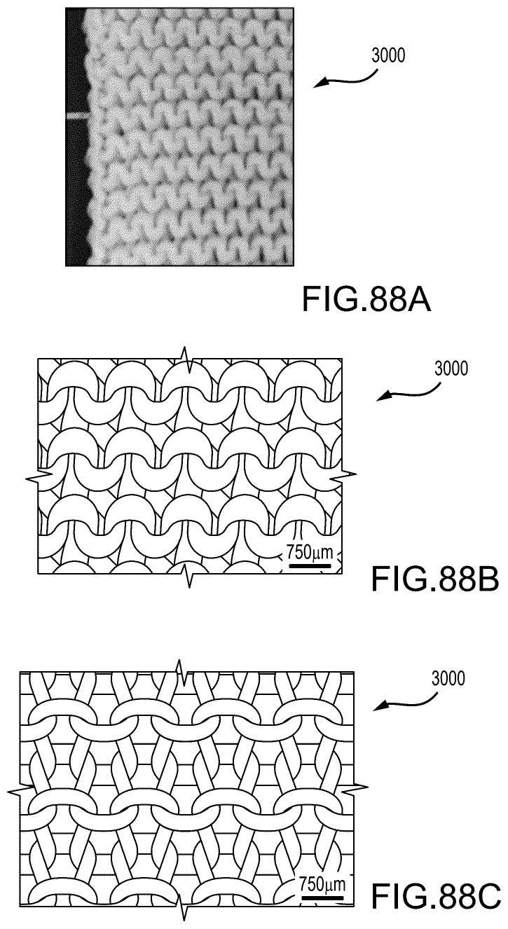

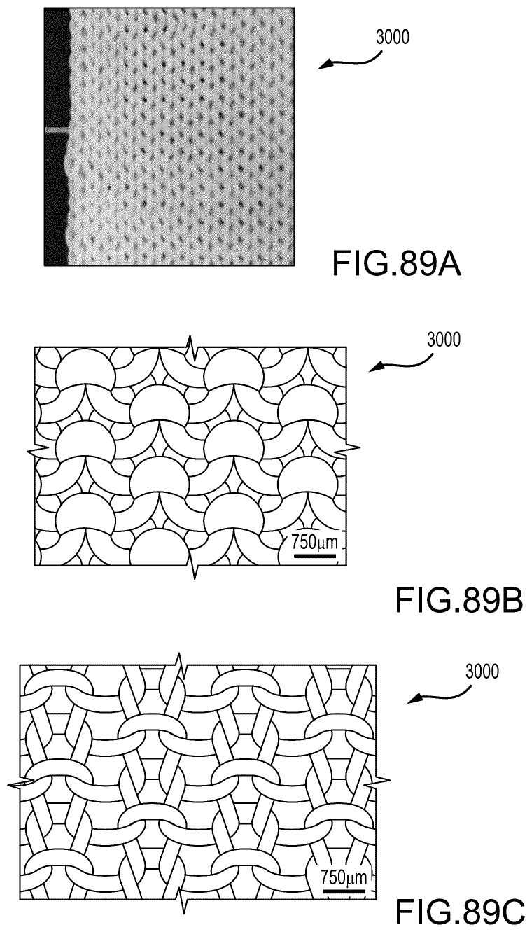

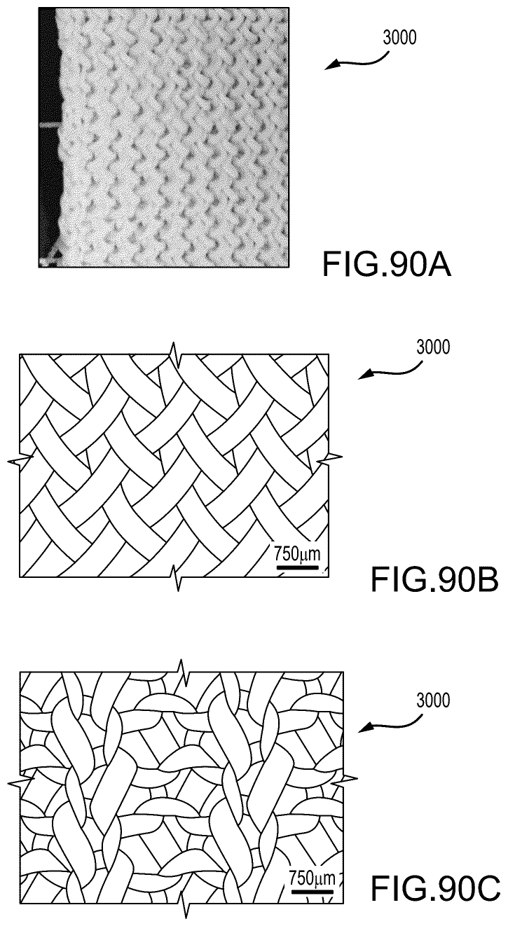

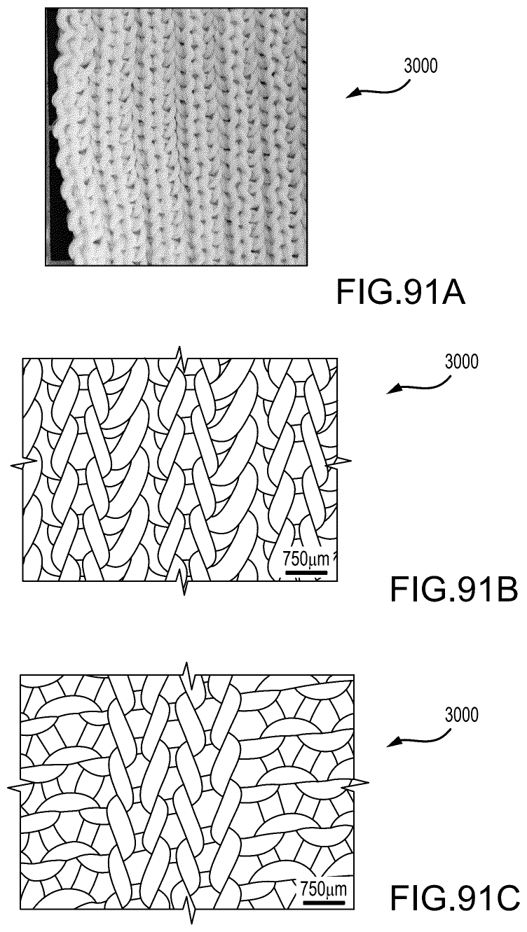

In some embodiments, the knit patterns may be different patterns in which the fiber(s) is/are interwoven, may include a different tightness of the knit, or a combination thereof. In some examples, the implantable substrate 3000 may be provided with varying knit patterns such as those illustrated in FIGS. 87A, 88A, 89A, 90A, and 91A. In some examples, the implantable substrate 3000 may be provided with regions implementing the same knit structure, but the density or tightness of the knit may vary along the implantable substrate (e.g., FIG. 87B vs. FIG. 87C, FIG. 88B vs. FIG. 88C, FIG. 89B vs. FIG. 89C, FIG. 90B vs. FIG. 90C, or FIG. 91B vs. FIG. 91C). In another example, the implantable substrate 3000 may include a combination of different knits and different tightness of the various knits. It is also understood that the knits may incorporate knots or other features such as locking points in order to limit unravelling of the knit construct if the implantable substrate 3000 is cut or during bioabsorption.

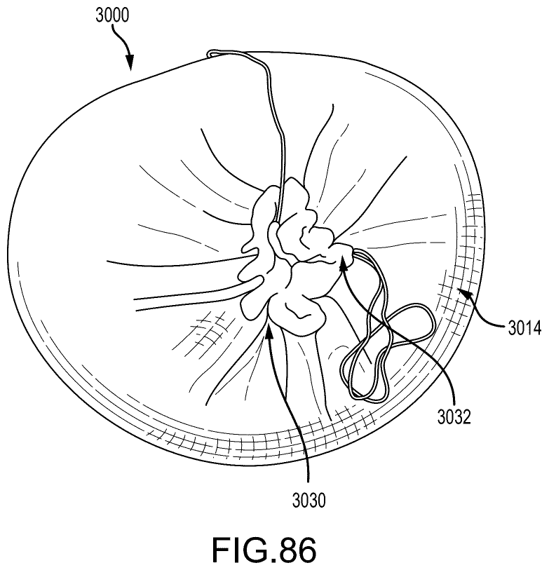

Referring to FIGS. 84-86, in some embodiments, the implantable substrate includes a cinch portion 3030. The cinch portion 3030 is positioned surrounding the access opening 3018. The cinch portion 3030 is operable to retain the implantable device 3100 when the implantable device 3100 is positioned in the cavity 3016. The cinch portion 3030 may be provided as an integral portion of the implantable substrate 3000. For example, the cinch portion 3030 may be a knitted region of the knitted substrate. In these embodiments, the cinch portion 3030 may be provided having a different knit pattern (e.g., a different knit or a different tightness of the knit as compared to other portions of the knitted substrate). Referring specifically to FIG. 86, in some embodiments, the implantable substrate 3000 may further include a closure member 3032 that is coupled to at least a portion of the knitted structure (e.g., the cinch portion 3030). The closure member 3032 is operable to maintain the access opening 3018 at a target size (e.g., diameter, area, etc.). In some embodiments, the closure member 3032 is provided to modify the size of the access opening 3018. For example, the closure member 3032 can be engaged with the implantable substrate 3000 (e.g., woven through the bioabsorbable fiber(s)) about at least a portion of the periphery of the knitted structure defining the access opening 3018 (e.g., the cinch portion). In such embodiments, the closure member 3032 may act as a drawstring. As the closure member 3032 is tensioned, the knitted structure defining the access opening 3018 is pulled together to decrease the size of the access opening 3018. This facilitates closure or partial closure of the access opening 3018 such that the implantable device 3100 is securely positioned and retained within the cavity 3016. This further allows for the implantable substrate 3000 to be pulled taught around the implantable device 3100, such that at least the anterior side 3012 is substantially free of folds, bends, wrinkles, overlap, creases, and so forth.

In some embodiments, the implantable substrate 3000 may include reinforcing members (not shown) that are positioned (e.g., woven into) with the implantable substrate 3000. The reinforcing member may be configured to limit expansion of the implantable substrate 3000 beyond a predetermined point. The reinforcing member may also be at least partially accessible to a surgeon such that the surgeon can manually set the predetermined stop points. In such embodiments, the reinforcing members may be actuated (e.g., tensioned) to define the stop points by acting as a drawstring. The reinforcing member may be secured (e.g., knotted) to limit the expansion of the implantable substrate 3000 at the reinforcing member as determined by the reinforcing member.

Various material may be implemented, including but not limited to, glycolide and trimethylene carbonate (PGA:TMC), for the bioabsorbable fibers knitted to form the implantable substrate 3000. The implantable substrate 3000 is provided to fit tightly around the implantable device 3100. When the implantable device 3100 is a tissue expander, as the tissue expander inflates in-vivo, the knit of the implantable substrate 3000 expands with the tissue expander to maintain a tissue regeneration matrix about the tissue expander. The tissue regeneration matrix as provided by the implantable substrate may be formed of and defined by a single fiber that is knit, formed of and defined by multiple fibers that are knit where the multiple fibers are each formed of the same material, or formed of and defined by multiple fibers that are knit where some of the multiple fibers are formed of different materials. In embodiments where the fibers are formed of different materials, the fibers may be provided with different degradation rates. This allows the implantable substrate 3000 to at least partially remain over time. The degradation rates can be tuned such that the implantable substrate 3000 degrades at a similar rate at which tissue regeneration occurs. Thus, as the tissue incorporates into the implantable substrates 3000, the fibers degrade at rates that facilitate continued incorporation and regeneration over time. Other materials that may be implemented for the bioabsorbable fibers include, but are not limited to, bioabsorbable monomers and bioabsorbable polymers and copolymers (including terpolymers) and blends that may include, in whole or in part, polyester amides, polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) poly(L-lactide-co-glycolide) and copolymeric variants, poly(D,L-lactide), poly(L-lactide), polyglycolide, poly(D,L-lactide-co-glycolide), poly L-lactide-co-glycolide), polycaprolactone, poly(lactide-co-caprolactone), poly(glycolid-caprolactone), poly(dioxanone), poly(ortho esters), poly(trimethylene carbonate), polyphosphazenes, poly anhydrides), poly(tyrosine carbonates) and derivatives thereof, poly(tyrosine ester) and derivatives thereof, poly(imino carbonates), poly(lactic acid-trimethylene carbonate), poly(glycolic acid-trimethylene carbonate), polyphosphoester, polyphosphoester urethane, poly(amino acids), poly(ethylene glycol) (PEG), copoly(ether-esters) (e.g. PEO/PLA), polyalkylene oxides such as polyethylene oxide), polypropylene oxide), poly(ether ester), polyalkylene oxalates, poly(aspirin), glycosamino glycan (GAG) and GAG derivatives, polylactic acid polymers (PLA), polyglycolic acid polymers (PGA), PGA/trimethylenecarbonate copolymers (PGA/TMC), poly-L-lactides (PLLA), polydiaoxanone (PDS), polyhydroxybutyrate, prolyl 4-hydroxylase (P4HB), copolymers of hydroxybutyrate and hydroxyvalerate, copolymers of lactic acid and E-caprolactone, biomolecules such as various forms of collagen, chitosan, alginate, fibrin, fibrinogen, cellulose, starch, collagen, polysaccharides, dextran, dextrin, fragments and derivatives of hyaluronic acid, heparin, fragments and derivatives of heparin, oxidized regenerated cellulose, elastin, chitosan, alginate, or combinations thereof. However, bioabsorbable fibers and materials, as used herein, comprise any material capable of biological absorption. In some embodiments, the implantable substrate 3000 is entirely bioabsorbable.

Referring to FIGS. 87A-91C, various knit patterns may be implemented to form the implantable substrate 3000. The various knit patterns are provided only as non-limiting examples, and, accordingly, other knit patterns may be implemented within the spirit of this disclosure. Referring specifically to FIGS. 87A-87C, the knit pattern shown includes that of a jersey knit (i.e., stocking stitch). Referring specifically to FIGS. 88A-88C, the knit pattern shown includes that of a garter knit. Referring specifically to FIGS. 89A-89C, the knit pattern shown includes that of a seed stitch knit. Referring specifically to FIGS. 90A-90C, the knit pattern shown includes that of a 1×1 rib stitch knit. Referring specifically to FIGS. 91A-91C, the knit pattern shown includes that of a 2×2 rib stitch knit. The various knits may be implemented in various portions of the implantable substrate 3000 in order to provide various properties to the implantable substrate 3000 (e.g., throughout the implantable substrate 3000 or in specific regions). A non-exclusive list of properties may include, stretch, stretch percentage, recovery, directionality of stretch, thickness, and so forth.

The fiber implemented may be selected from a variety of fibers, as discussed above. Each fiber may be a single filament or a plurality of filaments. Each filament in a fiber may include the same material or the fiber may be formed of filaments that are of different materials. In those embodiments implementing filaments of different materials, the filament types and ratio of filament types to each other may be selected for their various properties. For example, various materials may be selected for filaments implemented in a fiber to allow for different resorption rates in order to provide a specific degradation profile or for mechanical properties of the implantable substrate 3000. In some embodiments, the filaments may be bi-component and tri-component filaments of 2-3 different polymers within one cross-section of a filament.

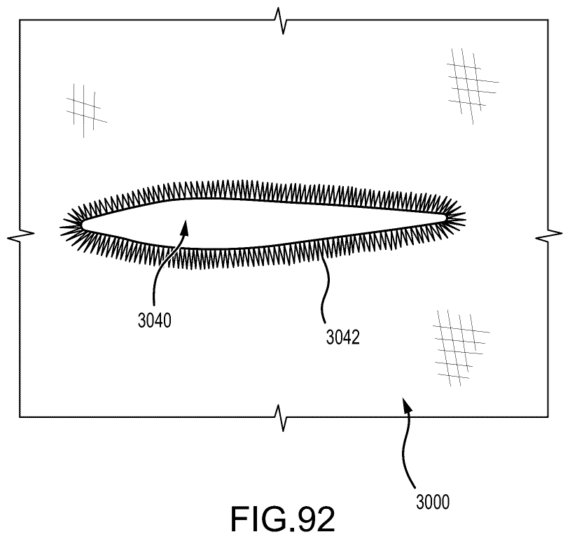

Referring to FIG. 92, the implantable substrate 3000 may define apertures 3040 through which components of the implantable device 3100 may extend. For example, the implantable device 3100 may include tissue anchoring members (not shown) that can extend through the apertures of the implantable substrate 3000. In these embodiments, the implantable substrate 3000 extends about and may substantially encapsulate the implantable device 3100 while facilitating direct securing of the implantable device directly to the tissue of the patient. The apertures 3040 may be reinforced using reinforcing features 3042 which may limit unraveling of the implantable substrate 3000. The reinforcing features 3042 may include sewing the edges of the implantable substrate 3000 proximate the apertures.

In some embodiments, the implantable substrate 3000 may include a flange (not shown), which is configured to limit exposure of the implantable device 3100, including the tissue anchoring members, from contacting the tissue of the patient on either an anterior or posterior side. The flange may also reduce the likelihood of puncturing the implantable device 3100 during implantation. The flange may be defined on the implantable substrate 3000 or may be coupled to the implantable substrate 3000. In some embodiments, the flange may extend partially or fully around the periphery of the posterior side 3014. In some embodiments, the flange is operable to extend beyond the tissue anchoring members of the implantable device 3100 when the implantable device 3100 is disposed within the cavity 3016. This facilitates coverage or isolation of the tissue anchoring members relative to at least one side of the tissue proximate the implantable device 3100. In embodiments in which the tissue anchoring members are positioned through at least a portion of the implantable substrate 3000 (e.g., respective apertures 3040), the tissue anchoring members may be separated or isolated from the tissue anterior to the tissue anchoring members while the tissue anchoring members are coupled to the tissue posterior to the tissue anchoring members. It is understood that in some embodiments, the flange may be shorter than the tissue anchoring members and are not fully covered. It is also understood that in some embodiments, a flange may not be implemented and the tissue anchoring members are not covered.

In some embodiments, the implantable device 3100 may be self supporting. For example, the tissue anchoring members are understood to support the implantable device 3100 (e.g., tissue expander, permanent implant, etc.) itself and the flange and implantable substrate 3000 generally do not provide structural support of the weight of the implantable device when implanted. Upon implantation, the tissue anchoring members may be coupled (e.g., sutured) to the patient's tissue, in accordance with an embodiment. However, it is understood that various arrangements may be implemented. For example, in some embodiments, the tissue anchoring members may be coupled to the flange or otherwise to the implantable substrate 3000 and the implantable device 3100 (e.g., the tissue expander or a permanent implant) is supported relative to the patient by the implantable substrate 3000. In some embodiments, the tissue anchoring members may extend through the apertures 3040 but are not coupled to the patient's tissue, such that the implantable substrate 3000 supports the implantable device 3100 (e.g., the tissue expander or a permanent implant). In some embodiments, the implantable device 3100 (e.g., the tissue expander or a permanent implant) is supported by the implantable substrate 3000 (for example, in the cavity 3016). In some embodiments, the flange and the tissue anchoring members are both coupled (e.g., sutured) to the patient's tissue. It is understood that these various configurations may be implemented and combinations thereof, either for specific scenarios or by preference of the surgeon. It is understood that the shape and size of the flange may be provided in various configurations, and the flange does not of necessity have to be continuous or uniform, although it may be.

The implantable substrate 3000 defined by a knit structure as described herein may be desirable for the ability to provide a three-dimensional structure, the ability to conform to various shapes and sizes while minimizing or preventing folds, bends, wrinkles, overlap, and creases, the ability to tune the stretchability and conformability of the implantable substrate 3000 at various positions along the implantable substrate 3000 via incorporation of various knit patterns, and the ability to quickly and efficiently manufacture such implantable substrates via high-volume, continuous manufacturing processes.

In some embodiments, a method of manufacturing an implantable substrate includes knitting a knit substrate including a three-dimensional shape and has a bulk material stretchability, conformability, and/or expandability, the knit substrate defining a cavity and an access opening into the cavity. Knitting the knitted substrate includes knitting a first portion with a first knit pattern and a second portion with a second knit pattern. The method may further include forming a fiber including a plurality of filaments. The fiber and filaments may include those previously discussed, such as PGA:TMC. The method may further include coupling a closure member to the knit substrate proximate the opening.

### Hybrid Non-Woven and Knit Implantable Substrates

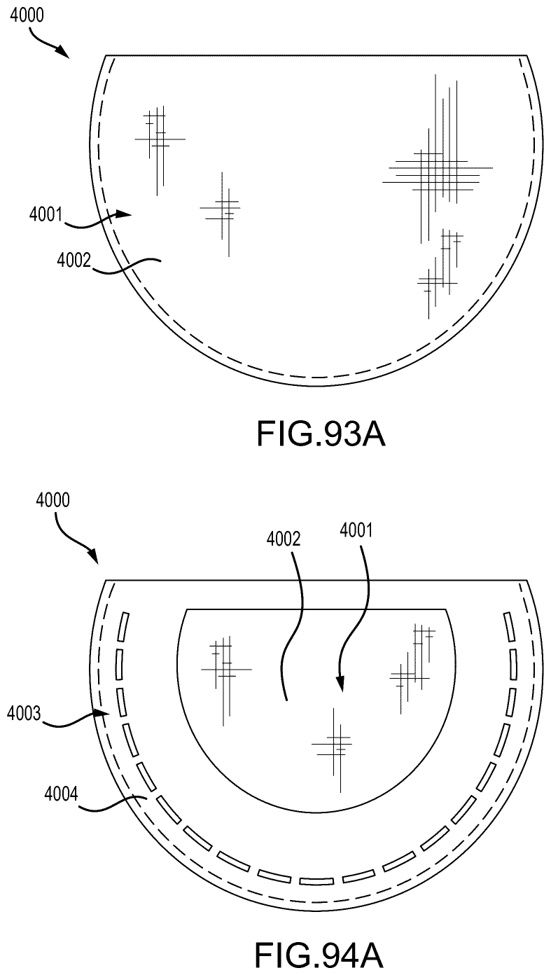

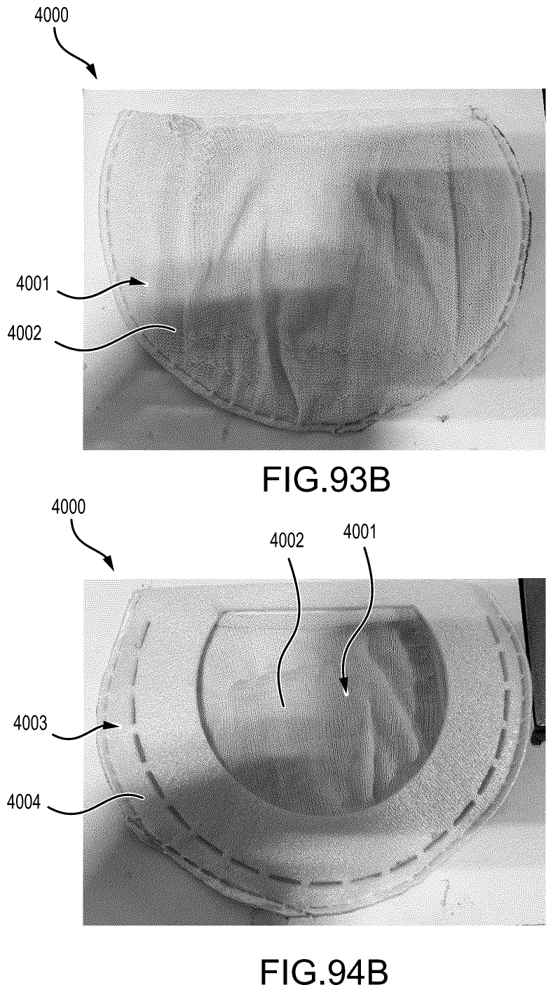

In some embodiments, a hybrid implantable substrate 4000 may be implemented. The hybrid implantable substrate 4000 is defined as an implantable substrate using two different materials, mediums, or configurations of a material to form the substrate. Referring to FIGS. 93-96, the hybrid implantable substrate 4000 includes a first tissue scaffold 4002 and second tissue scaffold 4004. The first tissue scaffold 4002 may be positioned on an anterior side 4001 and the second tissue scaffold 4004 may be position on a posterior side 4003. One of the first and second tissue scaffolds 4002, 4004 is formed of a non-woven material (e.g., having a non-woven structure) as previously discussed and the other of the first and second tissue scaffolds is formed of a knit material as previously discussed. FIG. 93A illustrates (FIG. 93B being an image thereof) the first tissue scaffold 4002 being formed of the knit material (e.g., warp-knit substrate or a weft-knit substrate). FIG. 94A illustrates (FIG. 94B being an image thereof) the second tissue scaffold 4004 being formed of the non-woven material.

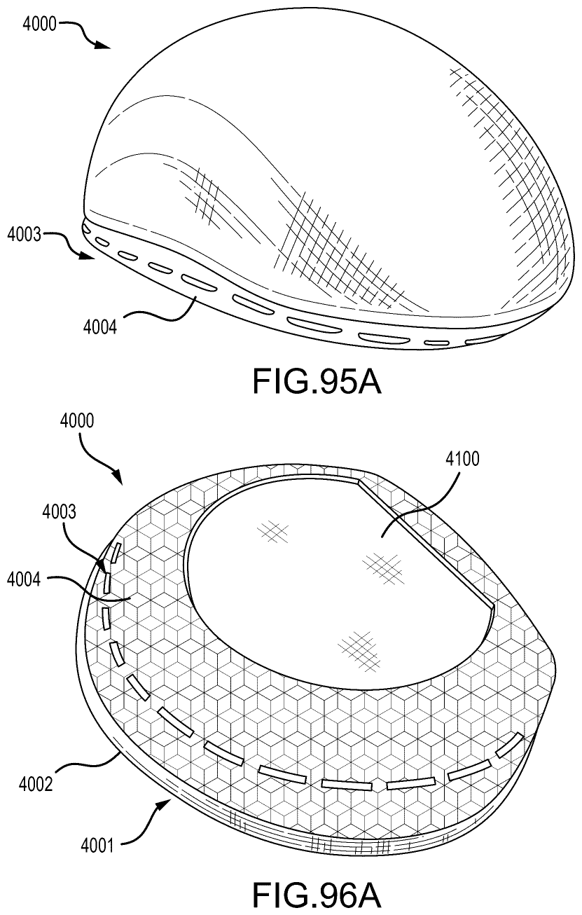

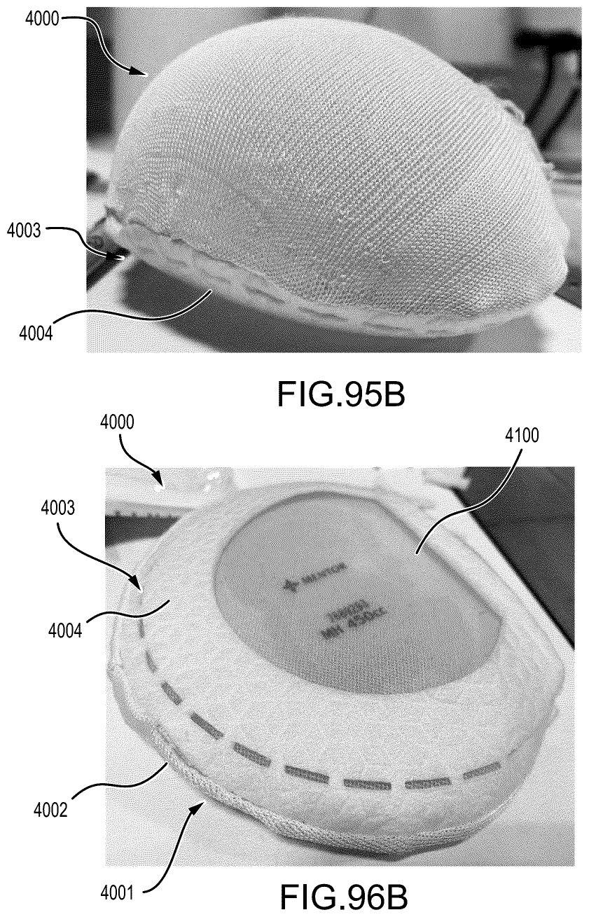

Turning first to a discussion of the first tissue scaffold 4002, the knit material of the first tissue scaffold 4002 may be provided as previously discussed, including the knit patterns facilitating expansions, materials for tissue incorporation and biosorption, and various other features. The knit patterns may be as described including one or more than one different pattern in the first tissue scaffold 4002. The first tissue scaffold 4002 may be provided as having a bulk two-dimensional structure (e.g., lies substantially flat on a flat surface) when in an unexpanded configuration or may have a bulk three-dimensional structure when in an unexpanded configuration. Referring specifically to FIGS. 93A, 93B, 95A, and 95B, the first tissue scaffold 4002 includes a knit pattern that facilitates expansion or stretch. For example, FIG. 93A illustrates the hybrid implantable substrate 4000 without an implantable device 4100 (e.g., a tissue expander or permanent implant) and thus the hybrid implantable substrate 4000 is shown in a collapsed or substantially flat configuration. FIG. 95A shows the hybrid implantable substrate 4000 with an implantable device 4100 positioned therein. The first tissue scaffold 4002 can be seen in an expanded or stretched state in order to accommodate the implantable device 4100.

Referring to FIG. 94A, 94B, 96A, and 96B, the second tissue scaffold 4004 may be provided as previously disclosed with respect to the non-woven embodiments. As shown in FIGS. 94A and 96A, the second tissue scaffold 4004 does not include stored length. However, it is understood that the second tissue scaffold 4004 may be provided with stored length, similar, for example to the embodiment shown in and discussed with respect to FIG. 68A. It is understood that the second tissue scaffold 4004 shown in FIGS. 94A and 96A is not to be considered limiting to the features of the second tissue scaffold 4004. For example, the features and geometries shown in any of the figures relating to the posterior side on an implantable substrate may be incorporated in the second tissue scaffold 4004 as appropriate. For example, the second tissue scaffold of 4004 of the hybrid implantable substrate 4000 may include various combinations of the features of the posterior side of any of FIG. 11, 18, 19, 25, 27-30, 42-54, or 56-79.

In some embodiments, the first tissue scaffold and second tissue scaffold 4002, 4004 are coupled to each other via an ultrasonic weld around at least a portion of the perimeter of the hybrid implantable substrate 4000. An opening 4006 may be maintained for insertion and manipulation of the implantable device 4100. It is understood that the first and second tissue scaffolds 4002, 4004 may be coupled in a variety of ways, including but not limited to sonic welding, thermal welding, an adhesive, bonding, suturing, sewing, and a fastening mechanism. As shown in FIG. 93, the coupling is a discontinuous ultrasonic weld. In some embodiments, excess portions of the first and/or second tissue scaffold 4002, 4004 may include a continuous weld/cut at an edge (e.g., adjacent to the ultrasonic weld coupling the first and second tissue scaffolds 4002, 4004) in order to remove excess material.

By having a non-woven material on the posterior side 4003 of the hybrid implantable substrate 4000, a surgeon is readily able to cut through the second tissue scaffold 4004 in order to access anchoring tabs (not shown, see FIG. 51 for example) of the implantable device 4100. This also allows for flexibility on the location for positioning of the anchoring tabs. It is also understood that a warp-knit material on the posterior side 4003 may include at least some similar properties as discussed here. The non-woven material may also be on the anterior side 4001 of the hybrid implantable substrate 4000, a surgeon is also readily able to cut through the first tissue scaffold 40024004 in order to access anchoring tabs of the implantable device 4100 or anchor to the patient's tissue (not shown, see FIG. 51 for example) of the implantable device 4100.

As shown in FIGS. 93A-96B, the first and second tissue scaffolds 4002, 4004 include similar or matching outer perimeters. However, it is understood that the first and second tissue scaffolds 4002, 4004. For example, the first tissue scaffold 4002 may extend beyond the second tissue scaffold 4004, similar to embodiments shown in at least FIGS. 48-54 or other relevant figures.

Various embodiments of the hybrid implantable substrate 4000 are contemplated herein. For example, in some embodiment the hybrid implantable substrate 4000 includes the first tissue scaffold 4002 positioned on an anterior side and the second tissue scaffold 4004 is positioned on a posterior side. In these embodiments, the second tissue scaffold 4004 may include stored length or the second tissue scaffold may not include stored length. In some embodiments, the first tissue scaffold 4002 is positioned on a posterior side and the second tissue scaffold 4004 is positioned on an anterior side. In these embodiments, the second tissue scaffold 4004 may include stored length.

Methods of implantation and breast reconstructions are provided herein. However, it is understood that the devices described herein may be implemented in various procedures, using various methods, and used for different applications than breast reconstruction or augmentation and still remain within the scope of the present disclosure. Additionally, the implantable substrate discussed herein with respect to the methods is understood to include any of the disclosed embodiments or contemplated embodiments with the various features. For example, the implantable substrate may include non-woven embodiments, knit embodiments, or hybrid embodiments as previously discussed.

A method of implantation and/or reconstruction may optionally includes saturating an implantable substrate in sterile solution per institutional standard of care prior to use or it may be used dry. Preparation of the implantable substrate and optionally the implantable device (e.g., tissue expander or permanent implant) may be performed at a back table or in the sterile field for immediate implantation into the patient.

A first method relates specifically to implantation for breast reconstruction (and to the extent applicable, breast augmentation). The first method relates to pre-pectoral implantation.

For implantable substrate designs that don't implement a posterior feature, the implantable device (e.g., a tissue expander or breast implant) is positioned within the implantable substrate such that an anterior portion of the implantable device is covered by the implantable substrate. The implantable substrate can be fixated to the implantable device by passing anchoring tabs through the implantable substrate (e.g., through provided slits or through newly cut slits). When slits are not provided, the physician or assistant can cut tabs into the implantable substrate in order to accommodate the anchoring tabs of the implantable device. As discussed previously, narrow slits with tags (see FIG. 51) may be provided, where the physician or assistant is able to cut or otherwise disengage the tags in order tailor the slits to the implantable device. The anchoring tabs may then be sutured across the posterior side. The combined construct of the implantable substrate and implantable device is placed in the patient's mastectomy defect on top of the pectoralis muscle ensuring appropriate tissue apposition. The construct is trimmed (as needed), positioned, and fixated to the patient's chest wall without significant wrinkles, folds, and creases at any of the top, sides, and bottom using anchoring tabs and/or edges of the implantable substrate for stabilization and to limit malposition or migration.

For designs with a posterior feature (including a cinch-see FIG. 50), the implantable device is positioned between the anterior and posterior sides of the implantable substrate. The implantable substrate can be further fixated to the implantable device by passing anchoring tabs through implantable substrate provided slits or through newly cut slits (as previously discussed), enclosing or fixating the implantable substrate with anchoring tabs or other features, and/or suturing across the posterior section of the implantable substrate. The construct of the implantable substrate and implantable device is placed in the patient's mastectomy defect on top of the pectoralis muscle. The construct is trimmed (as needed), positioned, and fixated to the patient's chest wall without significant wrinkles, folds, and creases at any of the top, sides, and bottom using anchoring tabs and/or implantable substrate edges for stabilization and to limit malposition or migration.

For designs with a flange, in additional to what is discussed above, the implantable substrate can be further fixated to the implantable device by suturing the anchoring tabs to the flange. The flange is trimmed (as needed), positioned, and fixated to the patient's chest wall without significant wrinkles, folds, and creases at any of the top, sides, and bottom to further aid stabilization and to limit malposition or migration.

For designs with an extension portion for accommodating or mimicking an anterior upper pole (see for example, FIG. 48), in addition to what is discussed above, extension portion is trimmed (as needed), positioned, and fixated to the patient's chest wall without significant wrinkles, folds, and creases to prevent tissue adhesion directly above the upper pole of the implantable device. This helps to create a more natural look to the patient upper chest anatomy, softening the transition from tissue to endoprosthesis.

Alternatively to placing the implantable device in the implantable substrate exterior to the patient, the implantable substrate can first be positioned in the patient's mastectomy defect on top of the pectoralis muscle. The top and/or sides of the implantable substrate are fixated to the patient's chest wall as detailed above without significant wrinkles, folds, and creases. The implantable device is then placed between the implantable substrate and the pectoralis muscle. Any of the remaining edges or features of the implantable substrate are fixated to the patient's chest wall without significant wrinkles, folds, and creases.

In some embodiments, the method includes subpectoral use. The method would be similar as described above except the implantable substrate and implantable device are placed underneath the patient's pectoralis muscle and the device would be fixated to the pectoralis muscle to allow for some lower pole expansion.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A material comprising:
an implantable substrate defined by at least one inelastic filament, wherein the implantable substrate is porous and includes recoverable stored length when free of elastomeric materials.

2. The material of claim 1, wherein the implantable substrate includes from about 30% to about 160% stored length.

3. The material of claim 1, wherein the implantable substrate is at least biaxially expandable at room temperature and body temperature.

4. The material of claim 1, wherein the implantable substrate defines pleats.

5. The material of claim 4, wherein the pleats are positioned from about 0.00 mm to about 3.00 mm apart from each other.

6. The material of claim 4, wherein at least some of the pleats intersect.

7. The material of claim 4, wherein at least one of the pleats include a non-uniform pattern.

8. The material of claim 4, wherein the pleats define a network of pleats.

9. The material of claim 1, wherein the pleats are configured to provide the recoverable stored length such that the pleats are configured to substantially return to an original configuration.

10. The material of claim 9, wherein the pleats are configured to limit macro-wrinkling and macro-folding of the implantable substrate when at least a portion of the recoverable stored length is recovered.

11. The material of claim 1, wherein the at least one inelastic filament includes melt-formed continuous filaments intermingled to form a porous web, wherein the melt-formed continuous filaments are self-cohered to each other at multiple contact points.

12. The material of claim 11, wherein the melt-formed continuous filaments comprise at least one semi-crystalline polymeric component covalently bonded to or blended with at least one amorphous polymeric component.

13. The material of claim 12, wherein the melt-formed continuous filaments possess partial to full polymeric component phase immiscibility when in a crystalline state.

14. The material of claim 1, wherein the implantable substrate has a porosity greater than 70%.

15. The material of claim 1, wherein the implantable substrate includes a three-dimensional porous structure.

16. The material of claim 15, wherein the three-dimensional porous structure is at least 0.1 mm thick.

17. The material of claim 15, wherein the three-dimensional porous structure includes an interconnected pore structure through a thickness of the material.

18. The material of claim 1, wherein the material defines a three-dimensional shape.

19. The material of claim 18, wherein the three-dimensional shape is at least one of a tubular construct, a sphere, a hemisphere, a partial sphere, a spheroid, a hemispheroid, a partial spheroid, an ellipsoid, a hemi-ellipsoid, a partial ellipsoid, a cone, and a partial dome.

20. The material of claim 1, wherein the material defines a substantially planar structure.

21. The material of claim 1, wherein the material includes at least a first region and a second region, wherein the first region has less stored length than the second region.

22. The material of claim 1, wherein the material includes a first region and a second region, wherein the first region includes less stored length per cm² relative to the second region.

23. The material of claim 1, wherein the at least one inelastic filament is bioabsorbable.
